SEQUENCE LISTING (2 sequences)

SEQ ID NO: 1 - DNA, 21 bp, synthetic construct
  source 1..21: mol_type = other DNA
catcagccctaatccatctga

SEQ ID NO: 2 - DNA, 21 bp, synthetic construct
  source 1..21: mol_type = other DNA
cgcgactaacaatcaaagtga

(19) United States
(12) Patent Application Publication
(10) Pub. No.: US 2026/0232706 A1
(43) Pub. Date: Aug. 13, 2026

(54) 1H-PYRAZOLE ANALOGUES AND METHODS AND USES THEREOF

(71) Applicant: University of Manitoba, Winnipeg (CA)

(72) Inventors: Geoffrey K. G. TRANMER, Winnipeg (CA); Nitesh SANGHAI, Winnipeg (CA)

(73) Assignee: University of Manitoba, Winnipeg, MB (CA)

(21) Appl. No.: 19/122,506

(22) PCT Filed: Oct. 12, 2023
(86) PCT No.: PCT/CA2023/051352

§ 371 Date: Sep. 12, 2025

Related U.S. Application Data
(60) Provisional application No. 63/417,482, filed on Oct. 19, 2022.

(51) Int. Cl. A61K 31/69; A61P 21/00; A61P 25/28; A61P 39/06; C07F 5/02

(52) U.S. Cl. CPC A61K 31/69; A61P 21/00; A61P 25/28; A61P 39/06; C07F 5/025; C07F 5/027

(57) ABSTRACT

A compound having a structure of Formula (I), a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof. The compound/composition may be used for prevention and/or treatment of a disease, condition and/or disorder associated with oxidative stress, such as, but not limited to, neurodegeneration, aging, etc.

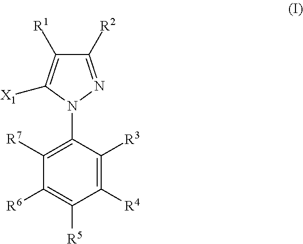

### FIELD

The present disclosure relates to compounds and methods and uses thereof. Specifically, the disclosure relates to 1H-pyrazole analogues and methods and uses thereof.

### BACKGROUND

Current evidence collectively demonstrates that amyotrophic lateral sclerosis (ALS) is a highly complex, multifactorial, life-threatening motor neurodegenerative disease, having different pathophysiology and progression due to a cascade of potential mechanisms. Unfortunately, the evidence for the mechanism of pathology for ALS is obscure and there is currently no cure for this disease.

Therapeutic strategies to combat this disease have minimal therapeutic effects and scope due to modest survival benefits, high economic burden, and synthetic hurdles. One such therapeutic strategy, Edaravone (EDR; 3-methyl-1-phenyl-2-pyrazolin-5-one), has been approved for ALS in several jurisdictions (e.g., by FDA (May 2017) for the treatment of ALS [1], and by Health Canada (October 2018) [2]).

The structure of EDR is shown below:

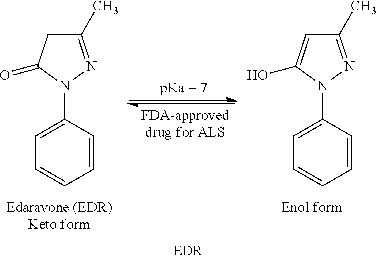

EDR, however, has limitations, including, for example, with respect to patient compliance, pharmacokinetics, and oral bioavailability.

There is a need for new EDR analogues and compositions thereof to provide a useful alternative to EDR.

The background herein is included solely to explain the context of the application. This is not to be taken as an admission that any of the material referred to was published, known, or part of the common general knowledge as of the priority date.

### SUMMARY

In an aspect, there is a compound having a structure of Formula I:

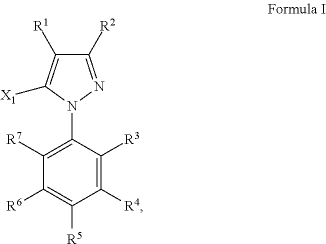

a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof,
wherein:
X₁ is selected from —BR⁸R⁹ or —BR¹⁰R¹¹R¹²;
R¹ to R⁷, R¹⁰, R¹¹, and R¹² are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, —BR¹³R¹⁴, —BR¹⁵R¹⁶R¹⁷, a substituted or unsubstituted carbocyclic group, a substituted or unsubstituted heterocyclic group, a substituted or unsubstituted aromatic group, or a substituted or unsubstituted heteroaromatic group, wherein R¹³ to R¹⁷ are each independently selected from H, a halo group, a hydroxyl group, a cyano group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group or R¹³ and R¹⁴ are taken together to form a substituted or unsubstituted carbocyclic group, or a substituted or unsubstituted heterocyclic group; and
R⁸ and R⁹ are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, a substituted or unsubstituted heteroaromatic group, or are taken together to form a substituted or unsubstituted carbocyclic group, or a substituted or unsubstituted heterocyclic group.

In another aspect, there is a pharmaceutical composition comprising the compound described herein.

In another aspect, there is a method for preventing and/or treating a disease, condition and/or disorder associated with oxidative stress, comprising administering to a mammal a therapeutically effective amount of the compound described herein or the composition described herein.

In yet another aspect, there is a use of a therapeutically effective amount of the compound described herein or the composition described herein for preventing and/or treating a disease, condition, and/or disorder associated with oxidative stress.

It is understood that one or more of the aspects described herein (and above) may be combined in any suitable manner. The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1 shows the chemical structure of Edaravone (EDR) in both the keto and the enol form.

FIG. 2 shows an example of a chemical structure of the 1H-pyrazole analogue(s) of EDR (B⁵-EDR) of FIG. 1.

FIG. 3 shows a schematic of an example B⁵-EDR analogue of FIG. 2, acting as a prodrug, to produce EDR in vivo via H₂O₂ metabolism.

FIGS. 4A-B shows examples of a chemical reaction between B⁵-EDR analogue(s) and H₂O₂ to produce Edaravone (EDR) and corresponding TLC plots. TLC A: SM=starting material; Mix=mixture of SM and CRM; CRM=Crude Reaction Mixture; TLC B: ISO=isolated product; Mix=mixture of isolated product and EDR; and EDR=Edaravone.

FIG. 5 shows examples of the structure of B⁵-EDR synthesized analogues of the present disclosure.

FIGS. 6A-B shows example graphs representing the percentage viability of primary neuronal cell cultures (PNCC) treated with EDR and B⁵-EDR analogues N-S-1-2, N-S-1-12, N-S-1-13, N-S-19, and N-S-1-21. Data are presented as a mean value±standard error of the mean (error bars); n=12 data points or sample size; data were analyzing using one-way AVONA followed by Dunnett's multiple comparisons test for statistical analysis (*P<0.05, **P=0.01 and ***P=0.001 versus control (DMSO)). Data are representative of two independent experiments, and each measurement or dose was tested six times.

FIGS. 7A-C show example graphs representing the percent viability of neuroblastoma-spinal cord NSC-34 cells treated with EDR and B⁵-EDR analogue NS-1-2, N-S-1-12, N-S-1-13, N-S-19, and N-S-1-21. Data are presented as a mean value±standard error of mean (error bars); where n=9 data points or sample size; data were analysed using one-way analysis of variance (ANOVA) followed by Dunnett's multiple comparison tests for statistical analysis (*P<0.05, versus control (DMSO)). Data are representative of three independent experiments, and each measurement or dose was tested in triplicate.

FIGS. 8A-B show example graphs representing the percent viability of neuroblastoma-spinal cord NSC-34 cells treated with EDR and B⁵-EDR analogues NS-1-2, N-S-1-12, N-S-1-13, N-S-19, and N-S-1-21, against hydrogen peroxide induced oxidative stress. Data are presented as a mean value±standard error of mean (error bars); where n=9 data points or sample size; data were analysed using one-way analysis of variance (ANOVA) followed by Dunnett's multiple comparison test for statistical analysis (####P<0.0001 control (DMSO), *P<0.05, **P<0.01, ***P<0.001, ****P<0.0001, ns=non-significant versus H₂O₂, DMSO). All the data are representative of three independent experiments, and each measurement or dose was tested in triplicate.

FIG. 9 shows an example schematic timeline of the development of ALS, including reference to representative humane endpoints, in the experimental mouse model (SOD1^{G37R}).

FIG. 10 shows example plots representing mean body weight for whole set of WG37R control and treated animal groups during the 14-day acute toxicity assessment. Data are presented as mean±SEM.

FIG. 11 shows example plots representing mean body weight for whole set of WG37R control and treated animal groups during the 120-days chronic toxicity assessment. Data are presented as mean±SEM.

FIG. 12 shows example photomicrographs of Hematoxylin and Eosin (H&E)-stained sections of two representative G37RWT male mice during the 14-day acute toxicity assessment. There were six mice (3 male and 3 female) in each group. Original magnification 10× (Scale bar represents 20 μm).

FIG. 13 shows example photomicrographs of Hematoxylin and Eosin (H&E)-stained sections of two representative G37RWT female mice during the 14-day acute toxicity assessment. There were six mice (3 male and 3 female) in each group. Original magnification 10× (Scale bar represents 20 μm).

FIG. 14 shows an example graph of survival (humane end point) in G37R (Line 42) mice. Data are presented as mean±SEM (n=10). n=Number of animals of the designated genotype. Differences in survival (days) were analysed using a two-tailed (unpaired t-test) to compare the relative differences between NS-1-2 (treatment) and vehicle treated G37R (Line 42) mice, with the significant level set at P<0.05. * P<0.05 versus control G37R.

FIG. 15 shows an example of a percent survival plot in G37R (Line 42) mice. Data are presented as mean±SEM (n=10); n=Number of animals of the designated genotype; data were analysed using a Kaplan-Meir Log-rank (Mantel-cox) test for percent survival plot (Humane end point) in Het G37R (Line 42) ALS model mice, for statistical significance (P<0.05).

FIG. 16 shows an example graph representing percentage weight loss (humane end point) based on the highest record weight in G37R (Line 42) mice. n=Number of animals of the designated genotype. Data are presented as mean±SEM (n=10). Percentage weight loss at humane endpoint was analysed using a two tailed (unpaired t-test) to compare the relative differences between NS-1-2 (treatment) and vehicle treated G37R (Line 42) mice, with the significance level set at P<0.05. **** P<0.0001 versus control G37R.

FIG. 17 shows an example graph representing age to disease onset (10% weight loss with muscle weakness). Data are presented as mean±SEM (n=10). n=Number of animals of the designated genotype. Disease onset was analysed using a two tailed (unpaired t-test) showing the number of days until disease onset defined as loss of 10% of body weight with muscle weakness, with significance set at P<0.05. **P<0.01 versus control G37R.

FIG. 18 shows an example graph representing age to disease onset (time to peak body weight (days)). Data are presented as mean±SEM (n=10). n=Number of animals of the designated genotype. Disease onset was analysed using a two tailed (unpaired t-test) showing the number of days until disease onset, which is retrospectively defined as the age at which the mouse reaches peak weight, with significance set at P<0.05. **P<0.01 versus control G37R.

FIG. 19 shows an example graph representing age to disease onset (time to peak body weight (days)). Data are presented as mean±SEM (n=10). n=Number of animals of the designated genotype. Data were analysed using a Kaplan-Meir Log-rank (Mantel-cox) test for determining the age of disease onset (age to reach peak body weight) in Het G37R (Line 42) ALS model mice, for statistical significance (**P<0.01).

### DETAILED DESCRIPTION OF CERTAIN ASPECTS

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the invention, the following terminology will be used.

It is also to be understood that the terminology used herein is for the purpose of describing particular aspects only and is not intended to be limiting. Any patent applications, patents, and publications are cited herein to assist in understanding the aspects described. All such references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supersede and/or take precedence over any such contradictory material.

In understanding the scope of the present application, the articles “a”, “an”, “the”, and “said” are intended to mean that there are one or more of the elements. Additionally, the term “comprising” and its analogues, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their analogues.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically, less than 0.1% by weight of non-specified component(s).

It will be understood that any component defined herein as being included may be explicitly excluded from the claimed invention by way of proviso or negative limitation.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The abbreviation, “e.g.” is derived from the Latin exempli gratia, and is used herein to indicate a non-limiting example. Thus, the abbreviation “e.g.” is synonymous with the term “for example.”

The phrase “at least one of” is understood to be one or more. The phrase “at least one of . . . and . . . ” is understood to mean at least one of the elements listed or a combination thereof, if not explicitly listed. For example, “at least one of A, B, and C” is understood to mean A alone or B alone or C alone or a combination of A and B or a combination of A and C or a combination of B and C or a combination of A, B, and C.

The terms “formulation” and “composition” may be used interchangeably.

The terms “therapeutically effective amount”, “effective amount” or “sufficient amount” may be considered a quantity sufficient, when administered to a subject, including a mammal (e.g. human), to achieve a desired result, for example an amount effective to relieve, to some extent, one or more of the symptoms of the disorder/disease being treated. Effective amounts of the compounds described herein may vary according to factors such as age, sex, and weight of the subject. Dosage or treatment regimes may be adjusted to provide the optimum therapeutic response, as is understood by a skilled person. Moreover, a treatment regime of a subject with a therapeutically effective amount may consist of a single administration, or alternatively comprise a series of applications. The length of the treatment period depends on a variety of factors, such as the compound used, the age of the subject, the concentration of the compound, the responsiveness of the patient to the compound, or a combination thereof. It will also be appreciated that the effective dosage of the compound used for the treatment may increase or decrease over the course of a particular treatment regime. Changes in dosage may result and become apparent by standard diagnostic assays known in the art.

As used herein, and unless otherwise specified, the terms “prevent,” “preventing” and “prevention” may be considered to be the prevention of the onset, recurrence or spread of a disease, condition and/or disorder or of one or more symptoms thereof. In certain embodiments, the terms refer to the treatment with or administration of a compound described herein, with or without one or more other additional active agent(s), prior to the onset of symptoms, particularly to a subject at risk of diseases, conditions, and/or disorders provided herein. The terms encompass the inhibition or reduction of a symptom of the particular disease. Subjects with familial history of a disease may be candidates for preventive regimes in certain embodiments. In this regard, the term “prevention” may be interchangeably used with the term “prophylactic treatment.”

“Administration” (e.g., “administering” a compound) in reference to a compound described herein may be considered to be the introduction of a compound into the system of the animal in need of treatment. When a compound of the disclosure is provided in combination with one or more other therapeutically active agent(s), “administration” and its variants can be each understood to include concurrent and sequential introduction of the compound or prodrug thereof and other agents. Administration “in combination with” one or more further therapeutic agents includes simultaneous (concurrent) and consecutive administration in any order.

“Amelioration” may be considered as a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Analogue” generally refers to a compound, for example, in which one or more individual atoms have been replaced, either with a different atom, or with a different functional group.

“Diseases, disorders and/or conditions associated with oxidative stress” refers to a disease, disorder and/condition, wherein at least part of the pathology/pathophysiology thereof is associated with/related to/caused by oxidative stress (definition provided below). Within the context of the present disclosure this includes, but is not limited to, neurodegenerative diseases, disorders and/or conditions, metabolic syndrome, cardiovascular diseases, disorders, and/or conditions, autoimmune diseases, disorders, and/or conditions, inflammatory lung diseases, disorders, and/or conditions, kidney diseases, disorders, and/or conditions, liver diseases, disorders, and/or conditions, digestive diseases, disorders, and/or conditions, aging, disorders and/or conditions, viral infectious diseases, disorders, and/or conditions, cancer, inflammation, sepsis, septic shock, systemic inflammatory response syndrome (SIRS). Examples of diseases, disorders and/or conditions associated with oxidative stress are provided in the description below.

“Inhibit” may be considered to be partially, substantially, or completely slowing, hindering, reducing, delaying or preventing. The terms inhibit, reduce, prevent, delay, and slow may be used interchangeably.

“Integer” may be considered any whole number, including zero.

“Oxidative stress” reflects an imbalance between the systemic manifestation of reactive oxygen species (ROS)/reactive nitrogen species (RNS) and antioxidants in favour of excessive generation of free radicals. This process leads to the oxidation of biomolecules with consequent loss of its biological functions and/or homeostatic imbalances, whose manifestation is the potential oxidative damage to cells and tissues. Accumulation of ROS/RNS can result in a number of deleterious effects such as lipid peroxidation, protein oxidation and DNA damage (including base damage and strand breaks). Further, some reactive oxidative species act as cellular messengers in redox signalling. Thus, oxidative stress can cause disruptions in normal mechanisms of cellular signalling.

“Parenteral” administration includes, e.g., subcutaneous (s.c.), intravenous (i.v.), intramuscular (i.m.), intraperitoneal (i.p.), or intrasternal injection, infusion techniques, or absorption through mucous membranes.

“Pharmaceutically acceptable” may be considered, for example, suitable for pharmaceutical use. In embodiments, a compound that is generally safe for administration to a mammal (e.g. human) according to established governmental standards, including those promulgated by the United States Food and Drug Administration.

“Pharmaceutically acceptable carrier” includes, carriers that are suitable for pharmaceutical use but is not limited to solvents, dispersion media, coatings, antibacterial agents, antifungal agents, isotonic and/or absorption delaying agents and the like. The use of pharmaceutically acceptable carriers is well known in the art.

“Prodrug”, as employed herein, may be considered to be a compound that is a drug precursor which, upon administration to a subject, undergoes chemical conversion by metabolic or chemical processes to yield a compound having a structure of Formula I or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Synergistic” or “synergistic therapeutic effect” refers to a greater-than-additive therapeutic effect which is produced by a combination of at least two agents, and which exceeds that which would otherwise result from the individual administration of the agents. For example, lower doses of one or more agents may be used in treating ALS, such as one or more therapeutic agents used to treat ALS, resulting in increased therapeutic efficacy and decreased side-effects.

“Subject” may be considered as any member of the animal kingdom, typically a mammal. The term “mammal” refers to any animal classified as a mammal, including humans and other higher primates. Typically, the mammal is human.

“Treatment” or “treat” may be considered to be the application of one or more specific procedures used for the cure or amelioration of a disease or condition. In certain embodiments, the specific procedure is the administration of one or more pharmaceutical agents.

The phrase “disease(s), disorder(s) and/or condition(s) associated with oxidative stress” is understood to mean at least one of a disease(s) associated with oxidative stress, a disorder(s) associated with oxidative stress, and a condition(s) associated with oxidative stress. Examples of disease(s), disorder(s) and/or condition(s) that are associated with oxidative stress are provided in the description below.

The compounds described herein may have asymmetric centers, chiral axes, and chiral planes (as described, for example, in: E. L. Eliel and S. H. Wilen, Stereo-chemistry of Carbon Compounds, John Wiley & Sons, New York, 1994, pages 1119-1190), and occur as racemates, racemic mixtures, scalemic mixtures, and as individual diastereomers, with all possible isomers (e.g. geometric) and mixtures thereof, including optical isomers, being included. In addition, the compounds described herein may exist as tautomers and both tautomeric forms are intended to be encompassed by the scope of the invention, even though only one tautomeric structure may be depicted. The compounds described herein may also include isotopologues (e.g. compounds that differ only in isotopic composition (number of isotopic substitutions), for example, CH₃, CH₂D, CHD₂) and isotopomers (e.g. isomers having the same number of each isotopic atom but differing in their positions).

It will be understood that any aspects/embodiments described as a compound, a pharmaceutically-acceptable salt, hydrate, solvate, tautomer, racemic mixture, scalemic mixture, enantiomer, diastereomer, isotopomer, isotopologue, prodrug, or combination thereof, means any one of a compound, a pharmaceutically-acceptable salt thereof, hydrate thereof, solvate thereof, tautomer thereof, racemic mixture thereof, scalemic mixture thereof, enantiomer thereof, diastereomer thereof, isotopomer thereof, isotopologue thereof, or prodrug thereof, in addition, or alternative, to various combinations thereof.

With respect to specific compound terminology, generally, reference to a certain element such as hydrogen or H is meant to, if appropriate, include all isotopes of that element.

Where the term “alkyl group” is used, either alone or within other terms such as “haloalkyl group” and “alkylamino group”, it encompasses linear or branched carbon radicals having, for example, one to about twenty carbon atoms or, in specific embodiments, one to about twelve carbon atoms. In other embodiments, alkyl groups are “lower alkyl” groups having one to about six carbon atoms. Examples of such groups include, but are not limited thereto, methyl, ethyl, n-propyl, isopropyl, n-butyl, isobutyl, sec-butyl, tert-butyl, pentyl, iso-amyl, hexyl and the like. In more specific embodiments, lower alkyl groups have one to four carbon atoms.

The term “alkenyl group” encompasses linear or branched carbon radicals having at least one carbon-carbon double bond. The term “alkenyl group” can encompass conjugated and non-conjugated carbon-carbon double bonds or combinations thereof. An alkenyl group, for example and without being limited thereto, can encompass two to about twenty carbon atoms or, in a particular embodiment, two to about twelve carbon atoms. In embodiments, alkenyl groups are “lower alkenyl” groups having two to about four carbon atoms. Examples of alkenyl groups include, but are not limited thereto, ethenyl, propenyl, allyl, propenyl, butenyl and 4-methylbutenyl. The terms “alkenyl group” and “lower alkenyl group”, encompass groups having “cis” and “trans” orientations, or alternatively, “E” and “Z” orientations.

The term “alkoxy group” encompasses linear or branched oxy-containing groups each having alkyl portions of, for example and without being limited thereto, one to about ten carbon atoms. In embodiments, alkoxy groups are “lower alkoxy” groups having one to six carbon atoms. Examples of such groups include methoxy, ethoxy, propoxy, butoxy and tert-butoxy. In certain embodiments, lower alkoxy groups have one to three carbon atoms. The “alkoxy” groups may be further substituted with one or more halo atoms, such as fluoro, chloro or bromo, to provide “haloalkoxy” groups. In other embodiments, lower haloalkoxy groups have one to three carbon atoms. Examples of such groups include fluoromethoxy, chloromethoxy, trifluoromethoxy, trifluoroethoxy, fluoroethoxy, and fluoropropoxy.

The term “alkylamino group” denotes amino groups which have been substituted with one alkyl group and with two alkyl groups, including terms “N-alkylamino” and “N,N-dialkylamino”. In embodiments, alkylamino groups are “lower alkylamino” groups having one or two alkyl groups of one to six carbon atoms, attached to a nitrogen atom. In other embodiments, lower alkylamino groups have one to three carbon atoms. Suitable “alkylamino” groups may be mono or dialkylamino such as N-methylamino, N-ethylamino, N,N-dimethylamino, N,N-diethylamino and the like.

The term “alkylaminoalkyl group” encompasses aminoalkyl groups having the nitrogen atom independently substituted with an alkyl group. In certain embodiments, the alkylaminoalkyl groups are “lower alkylaminoalkyl” groups having alkyl groups of one to six carbon atoms. In other embodiments, the lower alkylaminoalkyl groups have alkyl groups of one to three carbon atoms. Suitable alkylaminoalkyl groups may be mono or dialkyl substituted, such as N-methylaminomethyl, N, N-dimethyl-aminoethyl, N, N-diethylaminomethyl and the like.

The term “alkylaminoalkylamino group” denotes alkylamino groups which have been substituted with one or two alkylamino groups. In embodiments, there are C₁-C₃-alkylamino-C₁-C₃-alkylamino groups.

The terms “alkylcarbonyl group” denotes carbonyl groups which have been substituted with an alkyl group. In certain embodiments, “lower alkylcarbonyl group” has lower alkyl group as described above attached to a carbonyl group.

Where the term “alkylene group” is used, either alone or within other terms such as “haloalkylene group”, it encompasses linear or branched carbon radicals having, for example, one to about twenty carbon atoms or, in specific embodiments, one to about twelve carbon atoms. In other embodiments, alkylene groups are “lower alkylene” groups having one to about six carbon atoms. Examples of such groups include, but are not limited thereto, methylene, ethylene, n-propylene, isopropylene, n-butylene, isobutylene, sec-butylene, tert-butylene, pentylene, iso-amylene, hexylene and the like. In more specific embodiments, lower alkylene groups have one to four carbon atoms.

The term “alkylthio group” encompasses groups containing a linear or branched alkyl group, of one to ten carbon atoms, attached to a divalent sulfur atom. In certain embodiments, the lower alkylthio groups have one to three carbon atoms. An example of “alkylthio” is methylthio, (CH₃S—).

The term “alkynyl group” denotes linear or branched carbon radicals having at least one carbon-carbon triple bond. The term “alkynyl group” can encompass conjugated and non-conjugated carbon-carbon triple bonds or combinations thereof. Alkynyl group, for example and without being limited thereto, can encompass two to about twenty carbon atoms or, in a particular embodiment, two to about twelve carbon atoms. In embodiments, alkynyl groups are “lower alkynyl” groups having two to about ten carbon atoms. Some examples are lower alkynyl groups having two to about four carbon atoms. Examples of such groups include propargyl, butynyl, and the like.

The term “aminoalkyl group” encompasses linear or branched alkyl groups having one to about ten carbon atoms any one of which may be substituted with one or more amino groups. In some embodiments, the aminoalkyl groups are “lower aminoalkyl” groups having one to six carbon atoms and one or more amino groups. Examples of such groups include aminomethyl, aminoethyl, aminopropyl, aminobutyl and aminohexyl.

The term “aralkoxy group” encompasses oxy-containing aralkyl groups attached through an oxygen atom to other groups. In certain embodiments, aralkoxy groups are “lower aralkoxy” groups having optionally substituted phenyl groups attached to lower alkoxy group as described above.

The term “aralkyl group” encompasses aryl-substituted alkyl groups. In embodiments, the aralkyl groups are “lower aralkyl” groups having aryl groups attached to alkyl groups having one to six carbon atoms. In other embodiments, the lower aralkyl groups phenyl is attached to alkyl portions having one to three carbon atoms. Examples of such groups include benzyl, diphenylmethyl and phenylethyl. The aryl in said aralkyl may be additionally substituted with halo, alkyl, alkoxy, haloalkyl and haloalkoxy.

The term “aralkylamino group” denotes amino groups which have been substituted with one or two aralkyl groups. In other embodiments, there are phenyl-C₁-C₃-alkylamino groups, such as N-benzylamino. The “aralkylamino” groups may be further substituted on the aryl ring portion of the group.

The term “aromatic group” or “aryl group” means an aromatic group having one or more rings wherein such rings may be attached together in a pendent manner or may be fused. In particular embodiments, an aromatic group is one, two or three rings. Monocyclic aromatic groups may contain 4 to 10 carbon atoms, typically 4 to 7 carbon atoms, and more typically 4 to 6 carbon atoms in the ring. Typical polycyclic aromatic groups have two or three rings. Polycyclic aromatic groups having two rings typically have 8 to 12 carbon atoms, preferably 8 to 10 carbon atoms in the rings. Examples of aromatic groups include, but are not limited to, phenyl, naphthyl, tetrahydronaphthyl, indanyl, biphenyl, phenanthryl, anthryl or acenaphthyl.

The term “arylamino group” denotes amino groups which have been substituted with one or two aryl groups, such as N-phenylamino. The “arylamino” groups may be further substituted on the aryl ring portion of the group.

The term “arylalkenyl group” encompasses aryl-substituted alkenyl groups. In embodiments, the arylalkenyl groups are “lower arylalkenyl” groups having aryl groups attached to alkenyl groups having two to six carbon atoms. Examples of such groups include phenylethenyl. The aryl in said arylalkenyl may be additionally substituted with halo, alkyl, alkoxy, haloalkyl and haloalkoxy.

The term “arylalkynyl group” encompasses aryl-substituted alkynyl groups. In embodiments, arylalkynyl groups are “lower arylalkynyl” groups having aryl groups attached to alkynyl groups having two to six carbon atoms. Examples of such groups include phenylethynyl. The aryl in said aralkyl may be additionally substituted with halo, alkyl, alkoxy, haloalkyl and haloalkoxy. The terms benzyl and phenylmethyl are interchangeable.

The term “aryloxy group” encompasses optionally substituted aryl groups, as defined above, attached to an oxygen atom. Examples of such groups include phenoxy.

The term “arylthio group” encompasses aryl groups of six to ten carbon atoms, attached to a divalent sulfur atom. An example of “arylthio” is phenylthio. The term “aralkylthio group” encompasses aralkyl groups as described above, attached to a divalent sulfur atom. In certain embodiments there are phenyl-C₁-C₃-alkylthio groups. An example of “aralkylthio” is benzylthio.

The term “carbocyclic group” means a saturated or unsaturated carbocyclic hydrocarbon ring. Carbocyclic groups are not aromatic. Carbocyclic groups are monocyclic or polycyclic. Polycyclic carbocyclic groups can be fused, spiro, or bridged ring systems.

Monocyclic carbocyclic groups may contain 4 to 10 carbon atoms, typically 4 to 7 carbon atoms, and more typically 5 to 6 carbon atoms in the ring. Bicyclic carbocyclic groups may contain 8 to 12 carbon atoms, typically 9 to 10 carbon atoms in the rings.

The term “carbonyl group”, whether used alone or with other terms, such as “aminocarbonyl group”, denotes —(C═O)—.

The terms “carboxy group” or “carboxyl group”, whether used alone or with other terms, such as “carboxyalkyl group”, denotes —(C═O)—O—.

The term “cycloalkyl group” includes saturated carbocyclic groups. In certain embodiments, cycloalkyl groups include C₃-C₆ rings. In embodiments, there are compounds that include, cyclopentyl, cyclopropyl, and cyclohexyl.

The term “cycloalkenyl group” includes carbocyclic groups that have one or more carbon-carbon double bonds; conjugated or non-conjugated, or a combination thereof. “Cycloalkenyl” and “cycloalkyldienyl” compounds are included in the term “cycloalkenyl”. In certain embodiments, cycloalkenyl groups include C₃-C₆ rings. Examples include cyclopentenyl, cyclopentadienyl, cyclohexenyl and cycloheptadienyl. The “cycloalkenyl” group may have 1 to 3 substituents such as lower alkyl, hydroxyl, halo, haloalkyl, nitro, cyano, alkoxy, lower alkylamino, and the like.

The term “fused” means in which two or more carbons/member atoms are common to two adjoining rings, e.g., the rings are “fused rings”.

The term “halo” means halogens such as fluorine, chlorine, bromine or iodine atoms.

The term “haloalkyl group” encompasses groups wherein any one or more of the alkyl carbon atoms is substituted with halo as defined above. Specifically encompassed are monohaloalkyl, dihaloalkyl and polyhaloalkyl groups including perhaloalkyl. A monohaloalkyl group, for one example, may have either an iodo, bromo, chloro or fluoro atom within the group. Dihalo and polyhaloalkyl groups may have two or more of the same halo atoms or a combination of different halo groups. “Lower haloalkyl group” encompasses groups having 1-6 carbon atoms. In some embodiments, lower haloalkyl groups have one to three carbon atoms. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “heteroaromatic group” or “heteroaryl group” means an aromatic group having one or more rings wherein such rings may be attached together in a pendent manner or may be fused, wherein the aromatic group has at least one heteroatom. Monocyclic heteroaromatic groups may contain 4 to 10 member atoms, typically 4 to 7 member atoms, and more typically 4 to 6 member atoms in the ring. Typical polycyclic heteroaromatic groups have two or three rings. Polycyclic aromatic groups having two rings typically have 8 to 12 member atoms, more typically 8 to 10 member atoms in the rings. Examples of heteroaromatic groups include, but are not limited thereto, pyrrole, imidazole, thiazole, oxazole, furan, thiophene, triazole, pyrazole, isoxazole, isothiazole, pyridine, pyrazine, pyridazine, pyrimidine, triazine, indole, benzofuran, benzothiophene, benzimidazole, benzthiazole, quinoline, isoquinoline, quinazoline, quinoxaline and the like.

The term “heteroarylamino” denotes amino groups which have been substituted with one or two heteroaryl groups, such as N-thienylamino. The “heteroarylamino” groups may be further substituted on the heteroaryl ring portion of the group.

The term “heteroatom” means an atom other than carbon. Typically, heteroatoms are selected from the group consisting of sulfur, phosphorous, nitrogen and oxygen atoms. Groups containing more than one heteroatom may contain different heteroatoms.

The term “heterocyclic group” means a saturated or unsaturated ring structure containing carbon atoms and 1 or more heteroatoms in the ring. Heterocyclic groups are not aromatic. Heterocyclic groups are monocyclic or polycyclic. Polycyclic heterocyclic groups can be fused, spiro, or bridged ring systems. Monocyclic heterocyclic groups may contain 4 to 10 member atoms (i.e., including both carbon atoms and at least 1 heteroatom), typically 4 to 7, and more typically 5 to 6 in the ring. Bicyclic heterocyclic groups may contain 8 to 18 member atoms, typically 9 or 10 member atoms in the rings. Representative heterocyclic groups include, by way of example, pyrrolidine, imidazolidine, pyrazolidine, piperidine, 1,4-dioxane, morpholine, thiomorpholine, piperazine, 3-pyrroline and the like.

The term “heterogeneous group” means a saturated or unsaturated chain comprising carbon atoms and at least one heteroatom (e.g. ether group, ether group, etc.). Heterogeneous groups typically have 1 to 25 member atoms. More typically, the chain contains 1 to 12 member atoms, 1 to 10, and most typically 1 to 6. The chain may be linear or branched. Typical branched heterogeneous groups have one or two branches, more typically one branch. Typically, heterogeneous groups are saturated. Unsaturated heterogeneous groups may have one or more double bonds, one or more triple bonds, or both. Typical unsaturated heterogeneous groups have one or two double bonds or one triple bond. More typically, the unsaturated heterogeneous group has one double bond.

The term “hydrocarbon group” or “hydrocarbyl group” means a chain of 1 to 25 carbon atoms, typically 1 to 12 carbon atoms, more typically 1 to 10 carbon atoms, and most typically 1 to 8 carbon atoms. Hydrocarbon groups may have a linear or branched chain structure. Typical hydrocarbon groups have one or two branches, typically one branch. Typically, hydrocarbon groups are saturated. Unsaturated hydrocarbon groups may have one or more double bonds, one or more triple bonds, or combinations thereof. Typical unsaturated hydrocarbon groups have one or two double bonds or one triple bond; more typically unsaturated hydrocarbon groups have one double bond.

The term “hydroxyalkyl group” encompasses linear or branched alkyl groups having, for example and without being limited thereto, one to about ten carbon atoms, any one of which may be substituted with one or more hydroxyl groups. In embodiments, hydroxyalkyl groups are “lower hydroxyalkyl” groups having one to six carbon atoms and one or more hydroxyl groups. Examples of such groups include hydroxymethyl, hydroxyethyl, hydroxypropyl, hydroxybutyl and hydroxyhexyl.

The term “suitable substituent”, “substituent” or “substituted” used in conjunction with the groups described herein refers to a chemically and pharmaceutically acceptable group, i.e., a moiety that does not negate the therapeutic activity of the inventive compounds. It is understood that substituents and substitution patterns on the compounds of the invention may be selected by one of ordinary skill in the art to provide compounds that are chemically stable and that can be readily synthesized by techniques known in the art, as well as those methods set forth below. If a substituent is itself substituted with more than one group, it is understood that these multiple groups may be on the same carbon/member atom or on different carbons/member atoms, as long as a stable structure results. Illustrative examples of some suitable substituents include, halo, haloalkyl, perfluoroalkyl, perfluoroalkoxy, alkyl, alkenyl, alkynyl, hydroxy, oxo, mercapto, alkylthio, alkoxy, cycloalkyl, heterocyclyl, hydroxyalkyl, benzyl, carbonyl, aryl or heteroaryl, aryloxy or heteroaryloxy, aralkyl or heteroaralkyl, aralkoxy or heteroaralkoxy, HO—(C═O)—, amido, amino, alkyl- and dialkylamino, cyano, nitro, carbamoyl, alkylcarbonyl, alkoxycarbonyl, alkylaminocarbonyl, dialkylaminocarbonyl, arylcarbonyl, aryloxycarbonyl, alkylsulfonyl, and arylsulfonyl. Typical substituents include halo groups, hydroxyl groups, cyano groups, amino groups, hydrocarbon groups including alkyl groups such as methyl groups, substituted hydrocarbon groups such as benzyl, and heterogeneous groups including alkoxy groups such as methoxy groups, aromatic groups, or substituted aromatic groups.

When the term “unsaturated” is used in conjunction with any group, the group may be fully unsaturated or partially unsaturated. However, when the term “unsaturated” is used in conjunction with a specific group defined herein, the term maintains the limitations of that specific group. For example, an unsaturated “carbocyclic group”, based on the limitations of the “carbocyclic group” as defined herein, does not encompass an aromatic group.

The pharmaceutically acceptable salts of the compounds described herein include the conventional non-toxic salts of the compounds as formed, e.g., from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The pharmaceutically acceptable salts of the compounds described herein can be synthesized from the compounds described herein which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Compounds described herein may include pharmaceutically acceptable salts, hydrates, solvates, metabolites, and prodrugs of the compounds described herein and any suitable combinations thereof.

The compounds described herein are 1H-pyrazole analogue(s), a composition comprising at least one of the analogues, methods of administration thereof, and uses thereof are provided.

### 1H-Pyrazole Analogues of Formula I

In an embodiment, 1H-pyrazole analogue(s) are represented by a compound having a structure of Formula I:

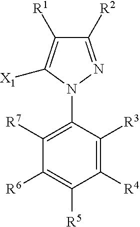

a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof,
wherein:
X₁ is selected from —BR⁸R⁹ or —BR¹⁰R¹¹R¹²;
R¹ to R⁷, R¹⁰, R¹¹, and R¹² are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, —BR¹³R¹⁴, —BR¹⁵R¹⁶R¹⁷, a substituted or unsubstituted carbocyclic group, a substituted or unsubstituted heterocyclic group, a substituted or unsubstituted aromatic group, or a substituted or unsubstituted heteroaromatic group, wherein R¹³ to R¹⁷ are each independently selected from H, a halo group, a hydroxyl group, a cyano group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group or R¹³ and R¹⁴ are taken together to form a substituted or unsubstituted carbocyclic group, or a substituted or unsubstituted heterocyclic group; and
R⁸ and R⁹ are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, a substituted or unsubstituted heteroaromatic group, or are taken together to form a substituted or unsubstituted carbocyclic group, or a substituted or unsubstituted heterocyclic group.

Additional embodiments are outlined below in i) to iii) for the compound having a structure of Formula I and these embodiments may be combined in any suitable permutation. With respect to one or more of the embodiments, it is understood that one or more atoms may be an isotope. In certain embodiments, it is understood that one or more H may be replaced with D (deuterium).

### i) Embodiments for X₁

a) X₁ is Selected from —BR*R⁹
Embodiments Whereby R⁸ and R⁹ do not Form a Ring Together:

In embodiments, R⁸ and R⁹ are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group.

In other embodiments, R⁸ and R⁹ are each independently selected from H, a halo group, a hydroxyl group, a cyano group, a nitro group, a sulfate group, a substituted or unsubstituted alkyl group, a substituted or unsubstituted alkenyl group, a substituted or unsubstituted alkynyl group, a substituted or unsubstituted alkoxy group, —C(O)H, a substituted carbonyl group, a substituted carboxyl group, —C(O)OH, a substituted or unsubstituted carbocyclic group, a substituted or unsubstituted heterocyclic group, a substituted or unsubstituted aromatic group, or a substituted or unsubstituted heteroaromatic group.

In other embodiments, R⁸ and R⁹ are each independently selected from H, a halo group, a hydroxyl group, a cyano group, a nitro group, a sulfate group, a substituted or unsubstituted alkyl group, a substituted or unsubstituted alkoxy group, —C(O)H, a substituted carbonyl group, a substituted carboxyl group, —C(O)OH, a substituted or unsubstituted heteroalkyl group, a substituted or unsubstituted cycloalkyl group, a substituted or unsubstituted heterocyclic group, a substituted or unsubstituted aromatic group, or a substituted or unsubstituted heteroaromatic group.

In another embodiment, R⁸ and R⁹ are each independently selected from H, a halo group, a hydroxyl group, a cyano group, a nitro group, a sulfate group, a substituted or unsubstituted alkyl group, a haloalkyl group, a substituted or unsubstituted alkylheteroalkyl group, a substituted or unsubstituted alkoxy group, —C(O)H, a substituted or unsubstituted alkylcarbonyl group, a substituted or unsubstituted alkylcarbonylalkyl group, a substituted or unsubstituted alkyl-C(O)O— group, a substituted or unsubstituted alkyl-C(O)O-alkylene group, a substituted or unsubstituted alkyl-O—C(O)— group, —C(O)OH, a substituted or unsubstituted cycloalkyl group, a substituted or unsubstituted heterocyclic group, a substituted or unsubstituted aromatic group, or a substituted or unsubstituted heteroaromatic group.

In another embodiment, R⁸ and R⁹ are each independently selected from H, a halo group, a hydroxyl group, a cyano group, a nitro group, a sulfate group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a substituted or unsubstituted C₁-C₆ cycloalkyl group, a substituted or unsubstituted C₁-C₆ heterocyclic group, a substituted or unsubstituted C₁-C₆ aromatic group, or a substituted or unsubstituted C₁-C₆ heteroaromatic group.

In another embodiment, R⁸ and R⁹ are each independently selected from H, a halo group, a hydroxyl group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a substituted or unsubstituted C₁-C₆ cycloalkyl group, a substituted or unsubstituted C₁-C₆ heterocyclic group, a substituted or unsubstituted phenyl group, a substituted or unsubstituted alkylheteroaryl group, a substituted or unsubstituted pyridinyl group, or a substituted or unsubstituted pyrrolyl group.

In another embodiment, R⁸ and R⁹ are each independently selected from H, a halo group, a hydroxyl group, a substituted or unsubstituted haloalkyl, a substituted or unsubstituted hydroxyalkyl, a substituted or unsubstituted cyanoalkyl, a substituted or unsubstituted alkenyl, a substituted or unsubstituted alkynyl, a substituted or unsubstituted cycloalkyl, a substituted or unsubstituted cycloalkenyl, a substituted or unsubstituted alkylcycloalkyl, a substituted or unsubstituted alkylcycloalkenyl, a substituted or unsubstituted heterocycloalkyl, a substituted or unsubstituted alkylheterocycloalkyl, a substituted or unsubstituted heterocycloalkenyl, a substituted or unsubstituted alkylheterocycloalkenyl, a substituted or unsubstituted aryl, a substituted or unsubstituted heteroaryl, a substituted or unsubstituted alkylaryl, a substituted or unsubstituted alkylheteroaryl, alkylene-O-alkyl, alkylene-O-cycloalkyl, alkylene-O-heterocycloalkyl, alkylene-O-alkylene-cycloalkyl, or alkylene-O-alkylene-heterocycloalkyl.

In another embodiment, R⁸ and R⁹ are each independently selected from a halo group, a hydroxyl group, or an alkoxy group. In specific embodiments, R⁸ and R⁹ are each independently selected from a fluoro group, a chloro group, a bromo group, a hydroxyl group, or an alkoxy group. In other embodiments, R⁸ and R⁹ are each independently selected from a fluoro group, a hydroxyl group, or an alkoxy group.

In another embodiment, R⁸ and R⁹ are each independently selected from a halo group or a hydroxyl group. In specific embodiments, R⁸ and R⁹ are each independently selected from a fluoro group, a chloro group, a bromo group, or a hydroxyl group. In other embodiments, R⁸ and R⁹ are each independently selected from a fluoro group or a hydroxyl group.

### Embodiments Whereby R⁸ and R⁹ Form a Ring Together

In embodiments, R⁸ and R⁹ are taken together to form a substituted or unsubstituted carbocyclic group, or a substituted or unsubstituted heterocyclic group.

In embodiments, R⁸ and R⁹ are taken together to form a substituted or unsubstituted a substituted or unsubstituted heterocyclic group. In embodiments, R⁸ and R⁹ are taken together to form a substituted or unsubstituted —O(C₂-C₈ alkylene)O— ring. In embodiments, R⁸ and R⁹ are taken together to form a substituted or unsubstituted —O(C₂-C₄ alkylene)O-ring. In other embodiments, R⁸ and R⁹ are taken together to form a substituted or unsubstituted —O(C₂-alkylene)O— ring. In other embodiments, R⁸ and R⁹ are taken together to form a substituted or unsubstituted —O(C₃-alkylene)O— ring. In other embodiments, R⁸ and R⁹ are taken together to form a substituted or unsubstituted —O(C₄-alkylene)O— ring. In embodiments, R⁸ and R⁹ are taken together to form —OCR₁R₂CR₃R₄O—, wherein R₁, R₂, R₃, and R₄ are each independently selected from any of the groups listed herein for R¹ to R⁷, R¹⁰, R¹¹, and R¹². In embodiments, R⁸ and R⁹ are taken together to form —OCH₂CH₂O—, —OC(CH₃)₂CH₂O—, or —OC(CH₃)₂C(CH₃)₂O—.

In embodiments, R⁸ and R⁹ are taken together to form a substituted or unsubstituted —O(C₁-C₂ alkylene)NH(C₁-C₂ alkylene)O— ring. In embodiments, R⁸ and R⁹ are taken together to form a substituted or unsubstituted —O(C₁-C₂ alkylene)N(alkyl)(C₁-C₂ alkylene)O— ring. In other embodiments, R⁸ and R⁹ are taken together to form a substituted or unsubstituted —O(C₂ alkylene)NH(C₂ alkylene)O— ring. In embodiments, R⁸ and R⁹ are taken together to form a substituted or unsubstituted —O(C₂ alkylene)N(alkyl)(C₂ alkylene)O— ring. In other embodiments, R⁸ and R⁹ are taken together to form —OCH₂CH₂NHCH₂CH₂O—, —OCH₂CH₂N(CH₃)CH₂CH₂O—, —OCH₂C(CH₃)₂NHCH₂CH₂O—, —OCH₂C(CH₃)₂N(CH₃)CH₂CH₂O—, —OCH₂C(CH₃)₂NHC(CH₃)₂CH₂O, —OC(CH₃)₂CH₂N(CH₃)C(CH₃)₂CH₂O—, or —OC(CH₃)₂C(CH₃)₂N(CH₃)C(CH₃)₂C(CH₃)₂O—.

b) X₁ is selected from —BR¹⁰R¹¹R¹²

In embodiments, R¹⁰, R¹¹ and R¹² are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic.

In other embodiments, R¹⁰, R¹¹ and R¹² are each independently selected from H, a halo group, a hydroxyl group, a cyano group, a nitro group, a sulfate group, a substituted or unsubstituted alkyl group, a substituted or unsubstituted alkenyl group, a substituted or unsubstituted alkynyl group, a substituted or unsubstituted alkoxy group, —C(O)H, a substituted carbonyl group, a substituted carboxyl group, —C(O)OH, a substituted or unsubstituted carbocyclic group, a substituted or unsubstituted heterocyclic group, a substituted or unsubstituted aromatic group, or a substituted or unsubstituted heteroaromatic group.

In other embodiments, R¹⁰, R¹¹ and R¹² are each independently selected from H, a halo group, a hydroxyl group, a cyano group, a nitro group, a sulfate group, a substituted or unsubstituted alkyl group, a substituted or unsubstituted alkoxy group, —C(O)H, a substituted carbonyl group, a substituted carboxyl group, —C(O)OH, a substituted or unsubstituted heteroalkyl group, a substituted or unsubstituted cycloalkyl group, a substituted or unsubstituted heterocyclic group, a substituted or unsubstituted aromatic group, or a substituted or unsubstituted heteroaromatic group.

In another embodiment, R¹⁰, R¹¹ and R¹² are each independently selected from H, a halo group, a hydroxyl group, a cyano group, a nitro group, a sulfate group, a substituted or unsubstituted alkyl group, a haloalkyl group, a substituted or unsubstituted alkylheteroalkyl group, a substituted or unsubstituted alkoxy group, —C(O)H, a substituted or unsubstituted alkylcarbonyl group, a substituted or unsubstituted alkylcarbonylalkyl group, a substituted or unsubstituted alkyl-C(O)O— group, a substituted or unsubstituted alkyl-C(O)O-alkylene group, a substituted or unsubstituted alkyl-O—C(O)— group, —C(O)OH, a substituted or unsubstituted cycloalkyl group, a substituted or unsubstituted heterocyclic group, a substituted or unsubstituted aromatic group, or a substituted or unsubstituted heteroaromatic group.

In another embodiment, R¹⁰, R¹¹ and R¹² are each independently selected from H, a halo group, a hydroxyl group, a cyano group, a nitro group, a sulfate group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a substituted or unsubstituted C₁-C₆ cycloalkyl group, a substituted or unsubstituted C₁-C₆ heterocyclic group, a substituted or unsubstituted C₁-C₆ aromatic group, or a substituted or unsubstituted C₁-C₆ heteroaromatic group.

In another embodiment, R¹⁰, R¹¹ and R¹² are each independently selected from H, a halo group, a hydroxyl group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a substituted or unsubstituted C₁-C₆ cycloalkyl group, a substituted or unsubstituted C₁-C₆ heterocyclic group, a substituted or unsubstituted phenyl group, a substituted or unsubstituted alkylheteroaryl group, a substituted or unsubstituted pyridinyl group, or a substituted or unsubstituted pyrrolyl group.

In another embodiment, R¹⁰, R¹¹ and R¹² are each independently selected from H, a halo group, a hydroxyl group, a substituted or unsubstituted haloalkyl, a substituted or unsubstituted hydroxyalkyl, a substituted or unsubstituted cyanoalkyl, a substituted or unsubstituted alkenyl, a substituted or unsubstituted alkynyl, a substituted or unsubstituted cycloalkyl, a substituted or unsubstituted cycloalkenyl, a substituted or unsubstituted alkylcycloalkyl, a substituted or unsubstituted alkylcycloalkenyl, a substituted or unsubstituted heterocycloalkyl, a substituted or unsubstituted alkylheterocycloalkyl, a substituted or unsubstituted heterocycloalkenyl, a substituted or unsubstituted alkylheterocycloalkenyl, a substituted or unsubstituted aryl, a substituted or unsubstituted heteroaryl, a substituted or unsubstituted alkylaryl, a substituted or unsubstituted alkylheteroaryl, alkylene-O-alkyl, alkylene-O-cycloalkyl, alkylene-O-heterocycloalkyl, alkylene-O-alkylene-cycloalkyl, or alkylene-O-alkylene-heterocycloalkyl.

In another embodiment, R¹⁰, R¹¹ and R¹² are each independently selected from a halo group, a hydroxyl group, or alkoxy group. In specific embodiments, R¹⁰, R¹¹ and R¹² are each independently selected from a fluoro group, a chloro group, a bromo group, a hydroxyl group, or an alkoxy group. In other embodiments, R¹⁰, R¹¹ and R¹² are each independently selected from a fluoro group, a hydroxyl group, or an alkoxy group.

In another embodiment, R¹⁰, R¹¹ and R¹² are each independently selected from a halo group or a hydroxyl group. In specific embodiments, R¹⁰, R¹¹ and R¹² are each independently selected from a fluoro group, a chloro group, a bromo group, or a hydroxyl group. In other embodiments, R¹⁰, R¹¹ and R¹² are each independently selected from a fluoro group or a hydroxyl group.

### ii) Embodiments for R¹ and R²

In embodiments, R¹ and R² are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group.

In a further embodiment, R¹ and R² are each independently selected from H, a halo group, a hydroxyl group, a cyano group, a substituted or unsubstituted amino group, a substituted or unsubstituted alkyl group, a substituted or unsubstituted alkenyl group, a substituted or unsubstituted alkynyl group, a substituted or unsubstituted alkoxy group, a substituted or unsubstituted carbocyclic group, a substituted or unsubstituted heterocyclic group, a substituted or unsubstituted aromatic group, or a substituted or unsubstituted heteroaromatic group.

In yet another embodiment, R¹ and R² are each independently selected from H, a halo group, a substituted or unsubstituted alkyl group, a substituted or unsubstituted heteroalkyl group, a substituted or unsubstituted cycloalkyl group, a substituted or unsubstituted heterocyclic group, a substituted or unsubstituted aromatic group, or a substituted or unsubstituted heteroaromatic group.

In another embodiment, R¹ and R² are each independently selected from H, a halo group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ cycloalkyl group, a substituted or unsubstituted C₁-C₆ heterocyclic group, a substituted or unsubstituted C₁-C₆ aromatic group, or a substituted or unsubstituted C₁-C₆ heteroaromatic group.

In another embodiment, R¹ and R² are each independently selected from H, a halo group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ cycloalkyl group, a substituted or unsubstituted C₁-C₆ heterocyclic group, a substituted or unsubstituted phenyl group, a substituted or unsubstituted pyridinyl group, or a substituted or unsubstituted pyrrolyl group.

In another embodiment, R¹ and R² are each independently selected from H, a halo group, a substituted or unsubstituted haloalkyl, a substituted or unsubstituted hydroxyalkyl, a substituted or unsubstituted cyanoalkyl, a substituted or unsubstituted alkenyl, a substituted or unsubstituted alkynyl, a substituted or unsubstituted cycloalkyl, a substituted or unsubstituted cycloalkenyl, a substituted or unsubstituted alkylcycloalkyl, a substituted or unsubstituted alkylcycloalkenyl, a substituted or unsubstituted heterocycloalkyl, a substituted or unsubstituted alkylheterocycloalkyl, a substituted or unsubstituted heterocycloalkenyl, a substituted or unsubstituted alkylheterocycloalkenyl, a substituted or unsubstituted aryl, a substituted or unsubstituted heteroaryl, a substituted or unsubstituted alkylaryl, a substituted or unsubstituted alkylheteroaryl, alkylene-O-alkyl, alkylene-O-cycloalkyl, alkylene-O-heterocycloalkyl, alkylene-O-alkylene-cycloalkyl, or alkylene-O-alkylene-heterocycloalkyl.

In another embodiment, R¹ is H or a substituted or unsubstituted alkyl group and R² is selected from H, a halo group, a hydroxyl group, a cyano group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group.

In a further embodiment, R¹ is H or a substituted or unsubstituted alkyl group and R² is selected from H, a halo group, a hydroxyl group, a cyano group, a substituted or unsubstituted amino group, a substituted or unsubstituted alkyl group, a substituted or unsubstituted alkenyl group, a substituted or unsubstituted alkynyl group, a substituted or unsubstituted alkoxy group, a substituted or unsubstituted carbocyclic group, a substituted or unsubstituted heterocyclic group, a substituted or unsubstituted aromatic group, or a substituted or unsubstituted heteroaromatic group.

In yet another embodiment, R¹ is H or a substituted or unsubstituted alkyl group and R² is selected from H, a halo group, a substituted or unsubstituted alkyl group, a substituted or unsubstituted heteroalkyl group, a substituted or unsubstituted cycloalkyl group, a substituted or unsubstituted heterocyclic group, a substituted or unsubstituted aromatic group, or a substituted or unsubstituted heteroaromatic group.

In another embodiment, R¹ is H or a substituted or unsubstituted C₁-C₆ alkyl group and R² is selected from H, a halo group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ cycloalkyl group, a substituted or unsubstituted C₁-C₆ heterocyclic group, a substituted or unsubstituted C₁-C₆ aromatic group, or a substituted or unsubstituted C₁-C₆ heteroaromatic group.

In another embodiment, R¹ is H or a substituted or unsubstituted C₁-C₆ alkyl group and R² is selected from H, a halo group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ cycloalkyl group, a substituted or unsubstituted C₁-C₆ heterocyclic group, a substituted or unsubstituted phenyl group, a substituted or unsubstituted pyridinyl group, or a substituted or unsubstituted pyrrolyl group.

In another embodiment, R¹ is H, —CH₃, —CH₂CH₃, or —CH₂CH₂CH₃ and R² is selected from H, F, Cl, CN, —CH₃, —CH₂F, —CHF₂, or —CF₃.

### iii) Embodiments for R³ to R⁷

In embodiments, R³ to R⁷ are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, —BR¹³R¹⁴, —BR¹⁵R¹⁶R¹⁷, a substituted or unsubstituted carbocyclic group, a substituted or unsubstituted heterocyclic group, a substituted or unsubstituted aromatic group, or a substituted or unsubstituted heteroaromatic group, wherein R¹³ to R¹⁷ are each independently selected from H, a halo group, a hydroxyl group, a cyano group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group or R¹³ and R¹⁴ are taken together to form a substituted or unsubstituted carbocyclic group, or a substituted or unsubstituted heterocyclic group.

In embodiments, R³ to R⁷ are each independently selected from H, a halo group, a hydroxyl group, a cyano group, a nitro group, a sulfate group, a substituted or unsubstituted amino group, a substituted or unsubstituted hydrocarbon group, a substituted or unsubstituted heterogeneous group, —BR¹³R¹⁴, —BR¹⁵R¹⁶R¹⁷, a substituted or unsubstituted carbocyclic group, a substituted or unsubstituted heterocyclic group, a substituted or unsubstituted aromatic group, or a substituted or unsubstituted heteroaromatic group, wherein R¹³ to R¹⁷ are each independently selected from H, a halo group, a hydroxyl group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group or R¹³ and R¹⁴ are taken together to form a substituted or unsubstituted carbocyclic group, or a substituted or unsubstituted heterocyclic group.

In embodiments, R³ to R⁷ are each independently selected from H, a halo group, a cyano group, a nitro group, a sulfate group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group.

In embodiments, R³ to R⁷ are each independently selected from H, a halo group, a cyano group, a nitro group, a sulfate group, a substituted or unsubstituted alkyl group, a substituted or unsubstituted alkenyl group, a substituted or unsubstituted alkynyl group, a substituted or unsubstituted alkoxy group, —C(O)H, a substituted carbonyl group, a substituted carboxyl group, —C(O)OH, a boronic acid group, a substituted or unsubstituted alkylboronate group, a substituted or unsubstituted carbocyclic group, a substituted or unsubstituted heterocyclic group, a substituted or unsubstituted aromatic group, or a substituted or unsubstituted heteroaromatic group.

In yet another embodiment, R³ to R⁷ are each independently selected from H, a halo group, a cyano group, a nitro group, a sulfate group, a substituted or unsubstituted alkyl group, a substituted or unsubstituted alkoxy group, —C(O)H, a substituted carbonyl group, a substituted carboxyl group, —C(O)OH, a boronic acid group, a substituted or unsubstituted alkylboronate group, a substituted or unsubstituted heteroalkyl group, a substituted or unsubstituted cycloalkyl group, a substituted or unsubstituted heterocyclic group, a substituted or unsubstituted aromatic group, or a substituted or unsubstituted heteroaromatic group.

In another embodiment, R³ to R⁷ are each independently selected from H, a halo group, a cyano group, a nitro group, a sulfate group, a substituted or unsubstituted alkyl group, a haloalkyl group, a substituted or unsubstituted alkylheteroalkyl group, a substituted or unsubstituted alkoxy group, —C(O)H, a substituted or unsubstituted alkylcarbonyl group, a substituted or unsubstituted alkylcarbonylalkyl group, a substituted or unsubstituted alkyl-C(O)O— group, a substituted or unsubstituted alkyl-C(O)O-alkylene group, a substituted or unsubstituted alkyl-O—C(O)— group, —C(O)OH, a boronic acid group, a substituted or unsubstituted alkylboronate group, a substituted or unsubstituted cycloalkyl group, a substituted or unsubstituted heterocyclic group, a substituted or unsubstituted aromatic group, or a substituted or unsubstituted heteroaromatic group.

In another embodiment, R³ to R⁷ are each independently selected from H, a halo group, a cyano group, a nitro group, a sulfate group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a boronic acid group, a substituted or unsubstituted C₁-C₆ alkylboronate group, a substituted or unsubstituted C₁-C₆ cycloalkyl group, a substituted or unsubstituted C₁-C₆ heterocyclic group, a substituted or unsubstituted C₁-C₆ aromatic group, or a substituted or unsubstituted C₁-C₆ heteroaromatic group.

In another embodiment, R³ to R⁷ are each independently selected from H, a halo group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a boronic acid group, a substituted or unsubstituted C₁-C₆ alkylboronate group, a substituted or unsubstituted C₁-C₆ cycloalkyl group, a substituted or unsubstituted C₁-C₆ heterocyclic group, a substituted or unsubstituted phenyl group, a substituted or unsubstituted alkylheteroaryl group, a substituted or unsubstituted pyridinyl group, or a substituted or unsubstituted pyrrolyl group.

In another embodiment, R³ to R⁷ are each independently selected from H, a halo group, a substituted or unsubstituted haloalkyl, a substituted or unsubstituted hydroxyalkyl, a substituted or unsubstituted cyanoalkyl, a substituted or unsubstituted alkenyl, a substituted or unsubstituted alkynyl, a substituted or unsubstituted cycloalkyl, a substituted or unsubstituted cycloalkenyl, a substituted or unsubstituted alkylcycloalkyl, a substituted or unsubstituted alkylcycloalkenyl, a substituted or unsubstituted heterocycloalkyl, a substituted or unsubstituted alkylheterocycloalkyl, a substituted or unsubstituted heterocycloalkenyl, a substituted or unsubstituted alkylheterocycloalkenyl, a substituted or unsubstituted aryl, a substituted or unsubstituted heteroaryl, a substituted or unsubstituted alkylaryl, a substituted or unsubstituted alkylheteroaryl, alkylene-O-alkyl, alkylene-O-cycloalkyl, alkylene-O-heterocycloalkyl, alkylene-O-alkylene-cycloalkyl, or alkylene-O-alkylene-heterocycloalkyl.

In another embodiment, R³, R⁴, R⁶, and R⁷ are each independently selected from H or a substituted or unsubstituted alkyl group and R⁵ is selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, —BR¹³R¹⁴, —BR¹⁵R¹⁶R¹⁷, a substituted or unsubstituted carbocyclic group, a substituted or unsubstituted heterocyclic group, a substituted or unsubstituted aromatic group, or a substituted or unsubstituted heteroaromatic group, wherein R¹³ to R¹⁷ are each independently selected from H, a halo group, a hydroxyl group, a cyano group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group or R¹³ and R¹⁴ are taken together to form a substituted or unsubstituted carbocyclic group, or a substituted or unsubstituted heterocyclic group.

In another embodiment, R³, R⁴, R⁶, and R⁷ are each independently selected from H or a substituted or unsubstituted alkyl group and R⁵ is selected from H, a halo group, a cyano group, a nitro group, a sulfate group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group.

In yet another embodiment, R³, R⁴, R⁶, and R⁷ are each independently selected from H or a substituted or unsubstituted alkyl group and R⁵ is selected from H, a halo group, a cyano group, a nitro group, a sulfate group, a substituted or unsubstituted alkyl group, a substituted or unsubstituted alkenyl group, a substituted or unsubstituted alkynyl group, a substituted or unsubstituted alkoxy group, —C(O)H, a substituted carbonyl group, a substituted carboxyl group, —C(O)OH, a boronic acid group, a substituted or unsubstituted alkylboronate group, a substituted or unsubstituted carbocyclic group, a substituted or unsubstituted heterocyclic group, a substituted or unsubstituted aromatic group, or a substituted or unsubstituted heteroaromatic group.

In yet another embodiment, R³, R⁴, R⁶, and R⁷ are each independently selected from H or a substituted or unsubstituted alkyl group and R⁵ is selected from H, a halo group, a cyano group, a nitro group, a sulfate group, a substituted or unsubstituted alkyl group, a substituted or unsubstituted alkoxy group, —C(O)H, a substituted carbonyl group, a substituted carboxyl group, —C(O)OH, a boronic acid group, a substituted or unsubstituted alkylboronate group, a substituted or unsubstituted heteroalkyl group, a substituted or unsubstituted cycloalkyl group, a substituted or unsubstituted heterocyclic group, a substituted or unsubstituted aromatic group, or a substituted or unsubstituted heteroaromatic group.

In yet another embodiment, R³, R⁴, R⁶, and R⁷ are each independently selected from H or a substituted or unsubstituted alkyl group and R⁵ is selected from H, a halo group, a cyano group, a nitro group, a sulfate group, a substituted or unsubstituted alkyl group, a haloalkyl group, a substituted or unsubstituted alkylheteroalkyl group, a substituted or unsubstituted alkoxy group, —C(O)H, a substituted or unsubstituted alkylcarbonyl group, a substituted or unsubstituted alkylcarbonylalkyl group, a substituted or unsubstituted alkyl-C(O)O— group, a substituted or unsubstituted alkyl-C(O)O-alkylene group, a substituted or unsubstituted alkyl-O—C(O)— group, —C(O)OH, a boronic acid group, a substituted or unsubstituted alkylboronate group, a substituted or unsubstituted cycloalkyl group, a substituted or unsubstituted heterocyclic group, a substituted or unsubstituted aromatic group, or a substituted or unsubstituted heteroaromatic group.

In another embodiment, R³, R⁴, R⁶, and R⁷ are each independently selected from H or a substituted or unsubstituted C₁-C₆ alkyl group and R⁵ is selected from from H, a halo group, a cyano group, a nitro group, a sulfate group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a boronic acid group, a substituted or unsubstituted C₁-C₆ alkylboronate group, a substituted or unsubstituted C₁-C₆ cycloalkyl group, a substituted or unsubstituted C₁-C₆ heterocyclic group, a substituted or unsubstituted C₁-C₆ aromatic group, or a substituted or unsubstituted C₁-C₆ heteroaromatic group.

In another embodiment, R³, R⁴, R⁶, and R⁷ are each independently selected from H or a substituted or unsubstituted C₁-C₆ alkyl group and R⁵ is selected from H, a halo group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a boronic acid group, a substituted or unsubstituted C₁-C₆ alkylboronate group, a substituted or unsubstituted C₁-C₆ cycloalkyl group, a substituted or unsubstituted C₁-C₆ heterocyclic group, a substituted or unsubstituted phenyl group, a substituted or unsubstituted alkylheteroaryl group, a substituted or unsubstituted pyridinyl group, or a substituted or unsubstituted pyrrolyl group.

In another embodiment, R³, R⁴, R⁶, and R⁷ are each independently selected from H or a substituted or unsubstituted C₁-C₆ alkyl group and R⁵ is selected from H, a halo group, a substituted or unsubstituted haloalkyl, a substituted or unsubstituted hydroxyalkyl, a substituted or unsubstituted cyanoalkyl, a substituted or unsubstituted alkenyl, a substituted or unsubstituted alkynyl, a substituted or unsubstituted cycloalkyl, a substituted or unsubstituted cycloalkenyl, a substituted or unsubstituted alkylcycloalkyl, a substituted or unsubstituted alkylcycloalkenyl, a substituted or unsubstituted heterocycloalkyl, a substituted or unsubstituted alkylheterocycloalkyl, a substituted or unsubstituted heterocycloalkenyl, a substituted or unsubstituted alkylheterocycloalkenyl, a substituted or unsubstituted aryl, a substituted or unsubstituted heteroaryl, a substituted or unsubstituted alkylaryl, a substituted or unsubstituted alkylheteroaryl, alkylene-O-alkyl, alkylene-O-cycloalkyl, alkylene-O-heterocycloalkyl, alkylene-O-alkylene-cycloalkyl, or alkylene-O-alkylene-heterocycloalkyl.

In another embodiment, R³, R⁴, R⁶, and R⁷ are each independently selected from H, —CH₃, —CH₂CH₃, or —CH₂CH₂CH₃ and R⁵ is selected from H, F, Cl, CN, —CH₃, —CH₂F, —CHF₂, or —CF₃.

In embodiments, the compound of Formula I may be selected from:

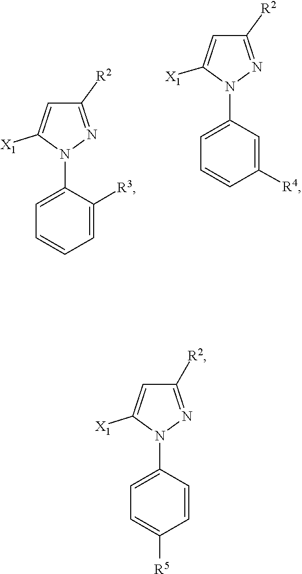

a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof. X₁ and R² to R⁵ are each independently selected from any one of the groups listed above under i) to iii).

In embodiments, the compound of Formula I may be selected from:

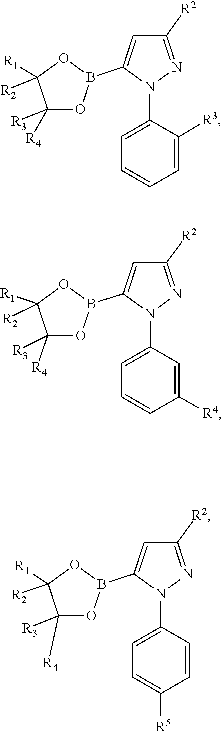

a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof. R₁ to R₄ and R² to R⁵ are each independently selected from any one of the groups listed above under i) to iii).

In embodiments, the compound of Formula I may be selected from:

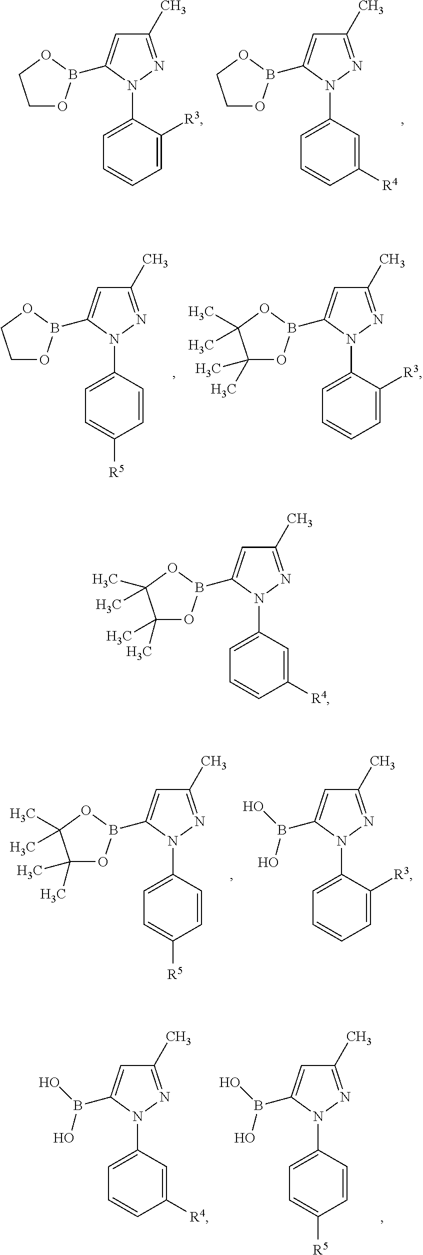

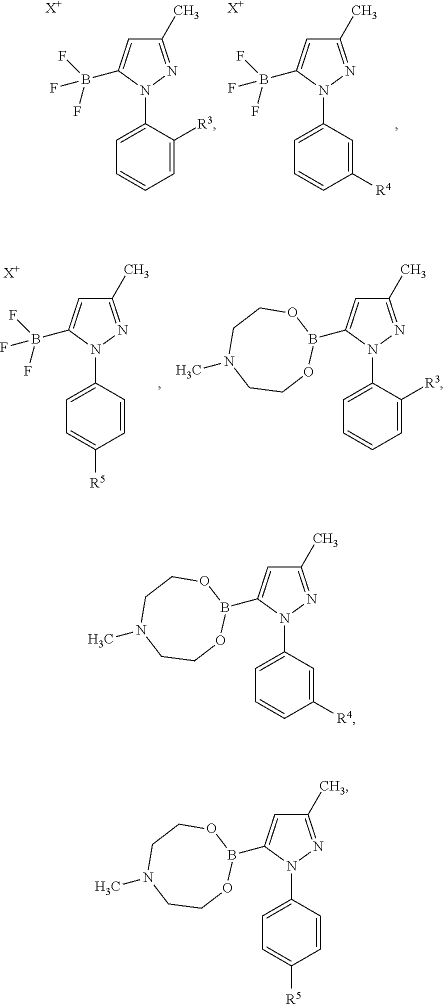

a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof. X⁺ is any suitable counterion (e.g. Na, K, etc.) and R³ to R⁵ are each independently selected from any one of the groups listed above under i) to iii). In additional embodiments, R³ to R⁵ are each independently selected from is selected from H, F, Cl, CN, —OH₃, —CH₂F, —CHF₂, or —CF₃.

In embodiments, the compound of Formula I may be selected from:

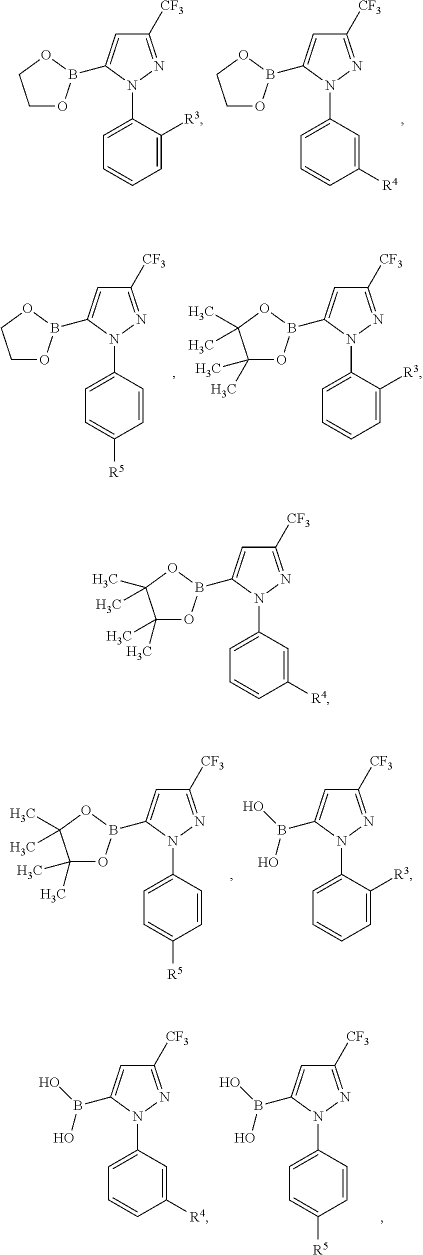

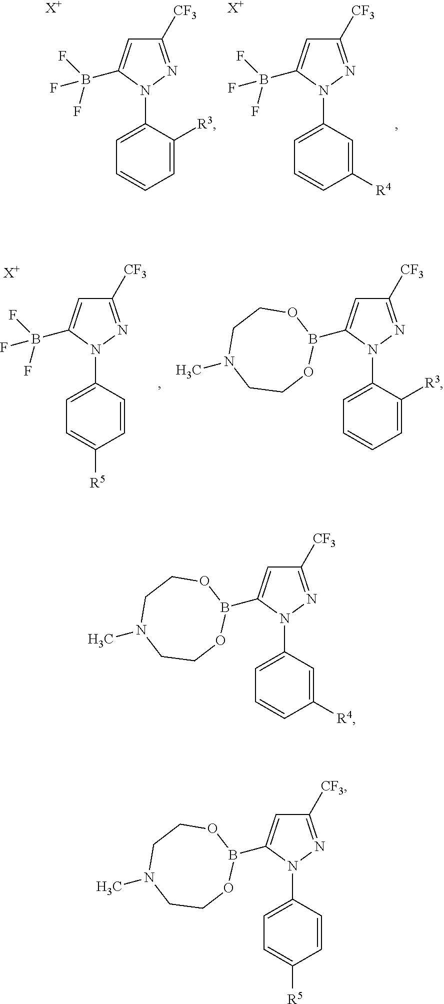

a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof. X⁺ is any suitable counterion (e.g. Na, K, etc.) and R³ to R⁵ are each independently selected from any one of the groups listed above under i) to iii). In additional embodiments, R³ to R⁵ are each independently selected from is selected from H, F, Cl, CN, —CH₃, —CH₂F, —CHF₂, or —CF₃.

In various embodiments, it is understood that the compounds described herein could be the single compound itself or a combination of compounds thereof, including a compound having a structure of Formula I, a pharmaceutically-acceptable salt thereof, hydrate thereof, solvate thereof, tautomer thereof, enantiomer thereof, diastereomer thereof, isotopomer thereof, isotopologue thereof, prodrug thereof. It could be, for example, a pharmaceutically acceptable salt thereof alone or in various combinations with the other compounds described herein. In embodiments, the compounds can be a racemic mixture or a scalemic mixture.

The compounds of Formula I, including a pharmaceutically-acceptable salt thereof, hydrate thereof, solvate thereof, tautomer thereof, enantiomer thereof, diastereomer thereof, isotopomer thereof, isotopologue thereof, prodrug thereof, or combination thereof, may be prepared by employing reactions and standard manipulations that are known in the literature or exemplified herein [46-48]. An example of a synthetic procedure for making the compounds of Formula I is shown as follows:

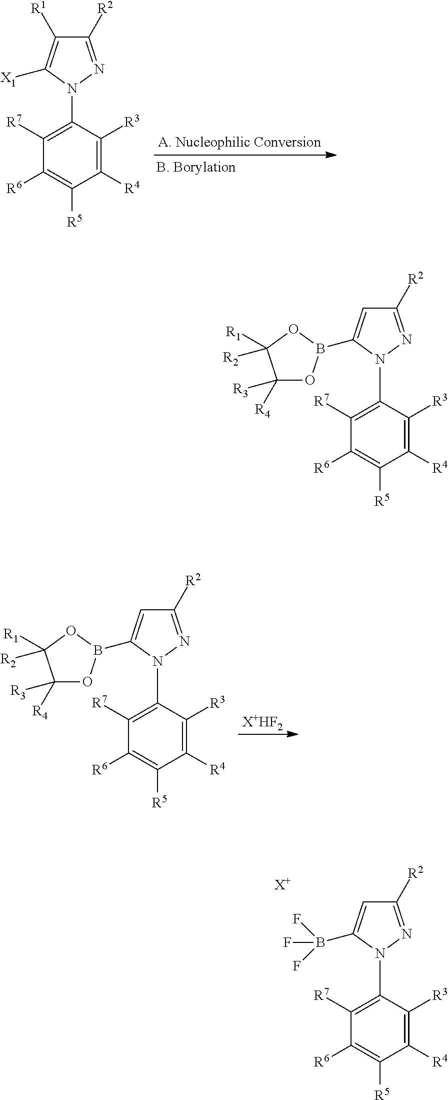

Another specific example is as follows:

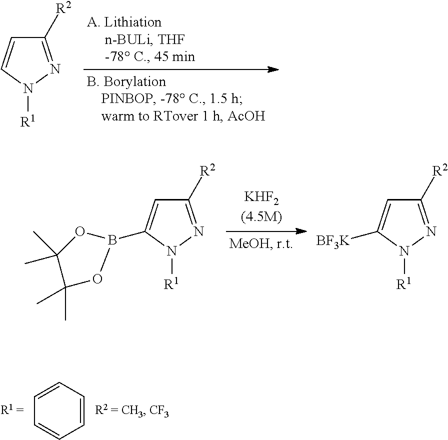

### Methods and Uses of 1H-Pyrazole Analogues and Compositions Thereof

In embodiments, the 1H-pyrazole analogue(s) described herein can act as a metal chelator. In this way, the 1H-pyrazole analogue(s) may be able to reduce genotoxicity induced by heavy metals. In embodiments, the heavy metals may be selected from arsenic trioxide, colloidal bismuth subcitrate, cadmium chloride, mercury chloride and/or lead chloride. As low doses of heavy metals are associated with, for example, neurodegenerative disease(s), the ability of the 1H-pyrazole analogue(s) described herein to chelate the heavy metals may provide an additional therapeutic benefit. To this regard and without being bound by theory, 1H-pyrazole analogue(s) described herein may form a reversible complex with metalloenzymes due to their lewis acid character and vacant p-orbital, and hence, could form a stable complex with metals like copper and zinc in the scaffolds of protein SOD1 (associated with neurodegenerative diseases like amyotrophic lateral sclerosis (ALS)), thereby making it more stable.

In other embodiments, the 1H-pyrazole analogue(s) described herein may act as an antioxidant. In this way, the 1H-pyrazole analogue(s) described herein are capable of reducing oxidative stress. This can play a role in, for example, alleviating symptoms associated with diseases, disorders and/or conditions associated with oxidative stress (described below).

Oxidative stress reflects an imbalance between the systemic manifestation of reactive oxygen species (hereinafter ROS)/reactive nitrogen species (hereinafter RNS) and antioxidants in favour of excessive generation of free radicals. This process leads to the oxidation of biomolecules with consequent loss of its biological functions and/or homeostatic imbalances, whose manifestation is the potential oxidative damage to cells and tissues. Accumulation of ROS/RNS can result in a number of deleterious effects such as lipid peroxidation, protein oxidation and DNA damage (including base damage and strand breaks). Further, some reactive oxidative species act as cellular messengers in redox signalling. Thus, oxidative stress can cause disruptions in normal mechanisms of cellular signalling.

“ROS” and “RNS” are the terms collectively describing free radicals and other non-radical reactive derivatives, which are also called oxidants. Radicals are less stable than non-radical species, although their reactivity is generally stronger. A molecule with one or more unpaired electron in its outer shell is called a free radical. Free radicals are formed from molecules via the breakage of a chemical bond such that each fragment keeps one electron, by cleavage of a radical to give another radical and, also via redox reactions.

Reduction of oxidative stress by the 1H-pyrazole analogue(s) described herein may be through several different mechanisms. For example, the 1H-Pyrazole analogue(s) may reduce oxidative damage/oxidative stress by scavenging ROS and/or RNS. Thus, as an antioxidant, the 1H-Pyrazole analogue(s) described herein may be able to scavenge ROS and/or RNS. In embodiments, the scavenging of the ROS and/or RNS can be the elimination of ROS and/or RNS. In other embodiments, the scavenging of the ROS and/or RNS can be reduction in the amount of ROS and/or RNS. Mechanisms of scavenging would be understood by those of skill in the art, and can include, for example, that which is carried out by donating a hydrogen atom to the ROS and converting the ROS into another molecule, like water, which is more stable. In some embodiments, the reduction of oxidative damage/oxidative stress is in vitro and in other embodiments, the reduction of oxidative damage/oxidative stress is in in vivo.

In embodiments, the ROS and/or RNS comprise free radicals and/or oxidants. The free radicals and/or oxidants include, but not limited to, hydroxyl radical (HO·), Superoxide radical anion radical (O2·-), nitric oxide (NO·), nitrogen dioxide (NO₂·), peroxyl (ROO·) and lipid peroxyl (LOO·), hydrogen peroxide (H₂O₂), ozone (O₃), singlet oxygen (¹O₂), hypochlorous acid (HOCl), hydroperoxide nucleophile (⁻OOH), nitrous acid (HNO₂), dinitrogen trioxide (N₂O₃), and lipid peroxide (LOOH). As would be understood, hydrogen peroxide (H₂O₂), ozone (O₃), singlet oxygen (¹O₂), nitrous acid (HNO₂), peroxynitrite (ONOO⁻), dinitrogen trioxide (N₂O₃), and lipid peroxide (LOOH) are not free radicals and generally called oxidants, but can easily lead to free radical reactions in living organisms.

In typical embodiments, the free radical and/or oxidant is hydrogen peroxide (H₂O₂). In specific embodiments, the 1H-pyrazole analogue(s) described herein capable of reacting with hydrogen peroxide to reduce the amount of ROS/oxidative stress in vitro and in other specific embodiments, the 1H-pyrazole analogue(s) described herein capable of reacting with hydrogen peroxide to reduce the amount of ROS/oxidative stress in vivo.

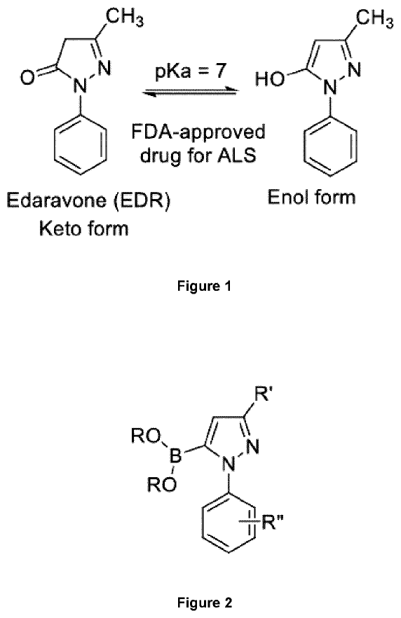

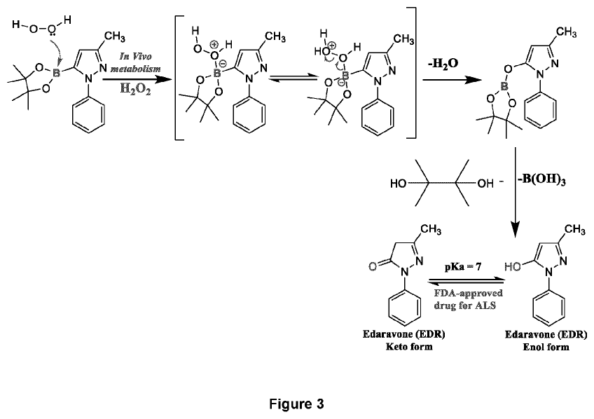

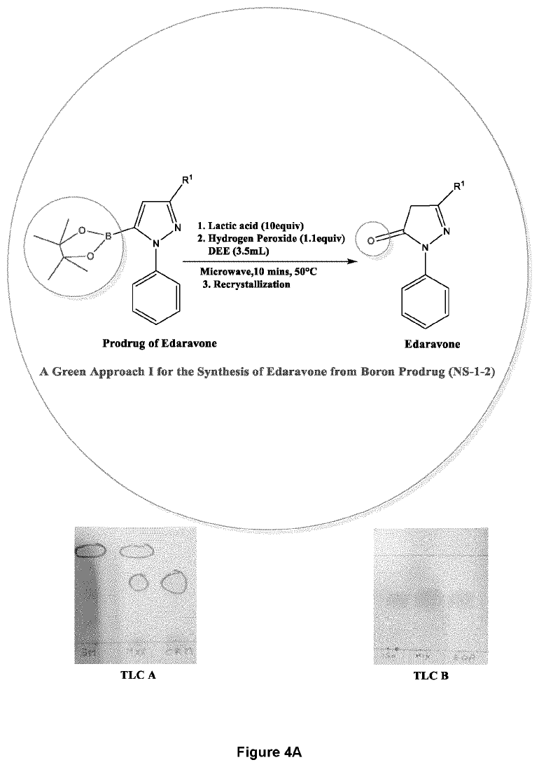

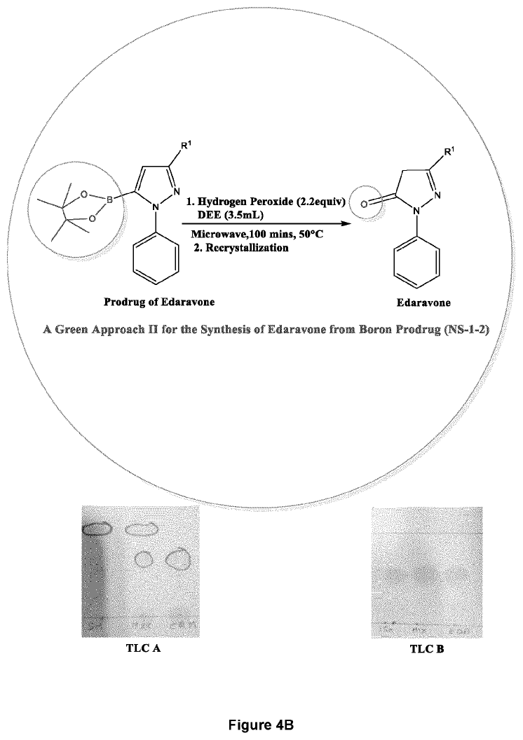

In embodiments, the 1H-pyrazole analogue(s) described herein can react with hydrogen peroxide to produce edaravone (3-methyl-1-phenyl-2-pyrazolin-5-one; EDR; FIG. 1), such that, in embodiments, the 1H-pyrazole analogue(s) described herein can act as a prodrug of EDR. Production of EDR in this way can be either in vitro or in vivo. With respect to prodrug function of the 1H-pyrazole analogue(s), in specific embodiments, derivatization of carbon-5 of EDR to produce the 1H-pyrazole analogue(s) described herein (FIG. 2), can allow for the 1H-pyrazole analogue(s) to be activated by ROS (e.g. H₂O₂) and release EDR/scavenge ROS (FIG. 3). In these embodiments, reaction with H₂O₂ or ROS in vivo can insert an oxygen atom at carbon-5 of EDR forming a new C—O bond in the 1H-pyrazole analogue(s) described herein. Following hydrolysis, an enol form of EDR can be produced such that the 1H-pyrazole analogue(s) serves as a prodrug for the in vivo generation of EDR (FIG. 3) directly within the cell, and/or site of oxidative stress. These features may be helpful for the 1H-pyrazole analogue(s) described herein to be useful as a therapeutic in preventing and/or treating diseases, disorders and/or conditions associated with oxidative stress (described below).

Without being bound to theory, the 1H-pyrazole analogues (i.e. B⁵-EDR analogues), by acting as prodrugs of EDR, are not only able to generate EDR directly within a cell, but, the 1H-pyrazole analogue(s) may also be able to mitigate oxidative stress by, for example, reacting with H₂O₂/ROS. In this way, the 1H-pyrazole analogue(s) may be able to treat and/or alleviate some of the symptomology caused by diseases, disorders and/or conditions associated with oxidative stress (described in more detail below) by, for example, targeting and/or eliminating molecules involved in oxidative stress pathways (e.g., H₂O₂/ROS). In other embodiments, the 1H-pyrazole analogue(s) described herein is a, antioxidant, and prodrug of EDR.

EDR has a low molecular weight (molecular weight of about 174.2 g/mol) biopharmaceutics classification system (BCS) class IV amphiphilic molecule due to poor aqueous solubility (about 1.85 mg/ml), permeability (Peff=about 3.18±0.0706*10-7 cm/s), short half-life of about 0.1 to about 5.16 hours with PKa value about 7 [3,22]. EDR is a redox regulator in cellular processes that acts by reducing both ROS/RNS free radicals (i.e. super oxide anions, hydroxyl radicals, singlet oxygen, peroxy radicals, hydrogen peroxide, peroxy nitrite) to their stable configuration state. Since EDR is an amphoteric molecule, it can reduce both aqueous and lipid soluble free radicals by donating one electron to complete their octet and making them electronically stable. This mechanism is called a single electron transfer process (SET) [18]. In addition, EDR is a substrate for P-glycoprotein (Pgp) efflux pump and metabolized pre-systematically by CYP3A4 enzymes. It also undergo extensive phase II metabolism, glucuronidation (about 68 to about 83%) by uridine glucorononosyl-transferase (UGT) enzymes and sulphation (about 5 to about 13%) by sulphotransferases forming pharmacologically inactive glucuronide and sulphate conjugates, which can lead to high passive permeability and low therapeutic concentration to the target side [3,23].

EDR exists as a solid in keto tautomer form, however, its tautomeric enol form exists as a highly unstable anion in aqueous solution, FIG. 1. The pKa of EDR is about 7.0 and its solubility in aqueous solution depends upon the pH. As the pH of the solution increases beyond about 8, EDR acts as an acid and furnishes hydronium ions to form its conjugate base, EDR anion. At physiological (pH about 7.4), about 71.5% of EDR exists as an anion while the remaining about 28.5% exists in neutral form [18]. The anion is capable of reducing radicals or even molecular oxygen by the single electron transfer process, forming stable EDR radicals which are stabilised by three resonance structures (enol, keto, and amine form) and subsequently form inactive EDR trimers in absence of oxygen, that appear as a yellow precipitate [24]. Decomposition of EDR takes place to form oxidative, stable products like OPB, 4-Oxo EDR, EDR peroxy radical, BPOH and phenyl hydrazine [25]. The poor oral bioavailability of EDR is due to its low aqueous solubility, low permeability, poor stability and extensive pre-systemic metabolism. Thus, analogues of EDR, that can act as prodrugs thereof, may improve these properties, as the compounds will not exist as keto-enol tautomers, as compared with EDR.

EDR diffuses into most organs to regulate the oxidation-reduction cycle including highly metabolic organs such as the brain and heart [19,20]. As a result, EDR was the first neuroprotective drug produced in Japan for the treatment of cerebral ischemic stroke [3] and has been approved for ALS in a number of jurisdictions (e.g. by FDA (May 2017) for the treatment of ALS [1], and by Health Canada (October 2018) [2]). EDR works as a free radical scavenger (antioxidant) and reduces oxidative stress in cells, the proposed MoA for helping patients recover from a stroke. In a randomized clinical trial, it was found that EDR delayed the progression of ALS in patients who are in the early-stages of the disease when administered the drug for 6 months or more [4]. Although the effects of EDR are modest, it remains one of the only therapeutics that has been shown clinically to improve patient outcomes. Furthermore, the exact cellular and molecular target of EDR is still not conclusively known, although an antioxidant-type pathway is proposed as one possible mechanism. EDR, however, has limitations, with respect to patient compliance, pharmacokinetics, oral bioavailability and can be unstable as an aqueous i.v. formulation. The recommended intravenous (i.v) dose for edaravone for ALS is 60 mg administered over a 60-minute period for 14 days, followed by 14-day drug free period [1] and is problematic for dosing in geriatric patients. For these patients, an oral route is highly preferred due to ease of administration, flexibility in dose, reduced prolonged hospitalisation, cost effectiveness, and improved quality of life. In specific embodiments, the 1H-pyrazole analogues described herein may be able to bypass some of the aforementioned limitations of EDR.

In embodiments, the 1H-pyrazole analogue(s) described herein may be useful for prevention and/or treatment of a disease, disorder and/or condition that is associated with oxidative stress. In other words, the 1H-pyrazole analogue(s) described herein may be useful for the prevention of, for the stabilization of, for lessening the severity of, for lessening the progression of and/or for the treatment of a disease, disorder and/or condition that is associated with oxidative stress. In embodiments, the 1H-pyrazole analogue(s) described herein may be useful for mitigating oxidative stress and/or neurotoxicity, resulting in, for example, neuroprotection and lessening of, for example, progression of the disease, disorder, and/or condition associated with oxidative stress. For example, in embodiments, the 1H-Pyrazole analogue(s) described herein are useful for mitigating oxidative stress and/or neurotoxicity, resulting in, neuroprotection and lessening of, for example, progression of a neurodegenerative disease, disorder, and/or condition associated with oxidative stress. In embodiments, the oxidative stress is caused by ROS and/or reactive nitrogen species RNS described herein.

In embodiments, the disease, condition, and/or disorder associated with oxidative stress is selected from neurodegenerative diseases, disorders and/or conditions, muscle diseases, disorders and/or conditions, vascular diseases, disorders and/or conditions, systemic inflammatory diseases, disorders, and/or conditions, local inflammatory diseases, disorders, and/or conditions, metabolic syndrome, cardiovascular diseases, disorders, and/or conditions, autoimmune diseases, disorders, and/or conditions, inflammatory lung diseases, disorders, and/or conditions, kidney diseases, disorders, and/or conditions, liver diseases, disorders, and/or conditions, digestive diseases, disorders, and/or conditions, aging, disorders and/or conditions, viral infectious diseases, disorders, and/or conditions, cancer, and sepsis/septic shock. In typical embodiments, the disease, condition, and/or disorder associated with oxidative stress comprises neurodegenerative diseases, disorders and/or conditions. In other typical embodiments, the neurodegenerative diseases, disorders and/or conditions are associated with motor dysfunction, and in further typical embodiments, the neurodegenerative diseases, disorders and/or conditions are associated with cognitive impairment.

The neurodegenerative diseases, disorders, and/or conditions with motor dysfunction, include, but are not limited to amyotrophic lateral sclerosis (ALS), Parkinson's disease, tardive dyskinesia (TD), epilepsy, ischemic stroke, cerebral ischemic injury, stroke and/or spinocerebellar degeneration. The neurodegenerative diseases, disorders, and/or conditions with cognitive impairment, include, but are not limited to, Alzheimer's disease, dementia, and/or Huntington's disease. In typical embodiments, the disease, disorder, and/or condition associated with oxidative stress is the neurodegenerative disease, disorder, and/or condition with motor dysfunction, and in other typical embodiments, the neurodegenerative disease, disorder, and/or condition with motor dysfunction is ALS. In some embodiments, ALS is familial ALS, and in other embodiments, ALS is sporadic ALS. In further embodiments, the ALS is caused by a mutation in the superoxide dismutase 1 (SOD1) gene or TARDBP gene.

ALS, also called Lou Gehrig's disease, is an idiopathic fatal neuromuscular disease of the motor neuron system that leads to impairment of voluntary skeletal muscles and eventually death from respiratory failure. The voluntary muscles weaken and become immobile; however, sensory systems and intellect are mostly unaffected. In simple terms, ALS is defined as a neurodegenerative disease and substantial research has shown that the misfolding and/or aggregation of specific proteins serve as a hallmark of this disease; ROS and RNS are contributors to this pathophysiology [3]. Variation in the redox cycle of normal cells can cause production of highly toxic ROS/RNS species and consequently, the death of neurons. An oxidizing cellular environment incites the formation of abnormal disulphide bonds and peroxynitrite nitrate tyrosine residue in proteins, leading to malformed protein aggregation or abnormal enzyme activities [5]. Various factors are involved in the onset and progression of ALS, however, the oxidative stress due to free radicals can be involved in the oxidation of hallmark proteins in neurodegenerative disorders, leading to the accumulation of misfolded proteins causing cell dysfunction and cell death [6].

ALS is largely categorized as two forms, sporadic (S)ALS (85-90% of cases) and genetically-linked familial (F)ALS (10-15% of cases). About 20% of FALS cases have been found to be caused by mutations in the superoxide dismutase 1 (SOD1) gene [7], with the pathophysiology of FALS and SALS having similar pathological and clinical mechanisms. The disease is thought to be caused by mutations in only one gene, SOD1, with more than 100 mutations in the SOD1 gene having been identified in patients suffering from ALS [8]. Substantial evidence has shown that mutant SOD1 proteins acquire toxic functions that causes the degeneration of neurons and has led to the breeding of transgenic mice that overexpress mutant SOD1-G93A and SOD1-G37R for use in ALS animal models. Oxidative stress induced SOD1 misfolding is a pathological marker for SOD1 mutations associated ALS [9]. Research findings suggest that pathological concentrations of mild oxidising agent like hydrogen peroxide (H₂O₂), which is a non-charged and non-ionising free radical initiator, is involved in the regulation of aggregation and toxicity of both wild type and mutant SOD1, causing the death of motor neurons [10-12]. H₂O₂ at pathological concentrations can induce the fibrillization and misfolding of SOD1 enzymes via oxidative modification of the amino acid Cys-111. Oxidized SOD1 has the potential to cause protein misfolding and exhibit toxicity that leads to the death of motor neurons [13,14].

Hydrogen peroxide (H₂O₂) is a paradoxical redox molecule, depending upon its concentration in living cells, it can cause cell signalling or cell death. At physiological concentration of about 1 to about 10 nM, it creates oxidative eustress and can initiate cellular processes such as proliferation, change in cell shape/size, etc. Higher concentrations in the range of >about 100 nM cause disruptive redox signalling, causing oxidative distress and therefore, the oxidation of biomolecules [15]. SOD1 regulates the level of H₂O₂ during normal physiological processes. It converts highly reactive and toxic superoxides to comparatively less reactive, uncharged, and freely diffusible hydrogen peroxide. Though SOD1's concentration in cells is low, its pathological concentration is about 10 to about 100 μM and can increase up to about 150 μM under conditions of high oxidative stress [16]. At concentrations of about 20 to about 200 nM H₂O₂, fibrillization of SOD1 proteins takes place and they gradually form amyloid fibrils that are rich in Beta-sheet conformations via oxidative modification of the amino acid Cys-111 to unstable and short-lived cysteine sulfenic acid (C-SOH) in neuronal cells. Sulfenic acid-modified SOD1 oligomers can cause TDP-43 re-distribution from nuclei to the cytoplasm, inducing the formation of SOD1/TDP-43 amyloid fibrils, a phenomenon observed during the progression of ALS [17]. Thus, can H₂O₂ contribute to the misfolding and toxicity of SOD1 in the nuclei of motor neurons causing their death [13]. Thus, mitigation of oxidative stress, and/or SOD1 oxidation/misfolding, may be a therapeutic strategy for the treatment ALS.

In embodiments, treatment of the disease, disorder and/or condition includes, for example, alleviation of disease, disorder, and/or condition progression, cure of the disease, disorder, and/or condition, prevention of morbidity, and/or prevention of recurrence, and may mean treatment for symptoms due to the disease, disorder, and/or condition described above. In embodiments, the treatment for symptoms includes, for example, suppression of progression of symptoms, alleviation of symptoms, cure of symptoms, prevention of the occurrence of symptoms, and/or prevention of recurrence of symptoms. The symptom is, for example, a dysfunction brought about by the disease, disorder, and/or condition associated with oxidative stress disease, such as a motor dysfunction or cognitive impairment in a neurodegenerative disease, disorder and/or condition. For example, in embodiments, use of the 1H-Pyrazole analogue(s) described herein may help alleviate the decline in motor function associated with ALS (e.g. the 1H-Pyrazole analogue(s) described herein may increase motor function in the subject with ALS).

In embodiments, the 1H-pyrazole analogue(s) described herein can impact a therapeutic index for ALS. In embodiments, the 1H-pyrazole analogue(s) described herein can improve the therapeutic index for ALS as compared to Edaravone (EDR). The therapeutic index can be a measure of a number of different symptoms of ALS, such as muscle weakness, muscle wasting (atrophy), weight loss (cachexia), muscle fasciculations, muscle spasticity, slowness of movement, poor balance, incoordination, alterations in vocal quality, dysarthria, dysphagia, incomplete eye closure, drooling, pseudobulbar affect, and/or premature death. In embodiments, the improved therapeutic index includes an improvement in any one or more of the above listed symptoms, when comparing the subject receiving the the 1H-pyrazole analogue(s) described herein as compared to the subject receiving EDR. In some embodiments, the improved therapeutic index is measured by increased survival/lifespan in the subject receiving the 1H-pyrazole analogue(s) as compared to the subject receiving Edaravone (EDR). In other embodiments, the improved therapeutic index is measured by increased motor function in the subject receiving the 1H-Pyrazole analogue(s) as compared to a subject receiving edaravone (EDR). In further embodiments, the improved therapeutic index is measured by a lower percentage of weight loss in the subject receiving the 1H-pyrazole analogue(s) as compared to a subject receiving edaravone (EDR).

In some embodiments, treatment and/or prevention of ALS with the 1H-pyrazole analogue(s) described herein ameliorates or eliminates of one or more of the following symptoms of ALS: muscle weakness, muscle wasting (atrophy), muscle fasciculations, muscle spasticity, slowness of movement, poor balance, incoordination, alterations in vocal quality, dysarthria, dysphagia, incomplete eye closure, drooling, pseudobulbar affect, and premature death. In other embodiments, treatment with the 1H-pyrazole analogue(s) described herein can prevent or delay the onset of one or more of the above-listed symptoms.

In some embodiments, improvement in the therapeutic index can be determined by detecting an improvement in the subject's symptoms compared to one or more of: (1) a baseline measurement or symptom level detected prior to or with commencement of treatment; (2) a measurement or symptom level from a control subject or a population of control subjects, wherein the control subjects exhibit one or more symptoms of ALS and (i) have not been administered the 1H-pyrazole analogue(s) described herein, or (ii) have been administered a control analogue(s); or (3) a standard.

In other embodiments, the 1H-pyrazole analogue(s) described herein may have improved pharmacokinetics as compared to edaravone (EDR). Since the pharmacokinetics may be governed by the 1H-pyrazole analogue(s), acting as prodrug, the 1H-pyrazole analogue(s) may possess better drug-like properties, as compared to, for example, EDR, such as greater lipophilicity, increased membrane permeability, decreased Pgp recognition, and longer half-life. In this way, a subject receiving the 1H-pyrazole analogue(s) described herein may benefit from the 1H-pyrazole analogue(s) described herein increased bioavailability in vivo (e.g., the 1H-pyrazole analogue(s) described herein have a longer half-half and/or is more resistant to degradation in vivo) as compared to EDR. Increased bioavailability of the 1H-pyrazole analogue(s) described herein can contribute to, for example, increased chelation of heavy metal(s), increased scavenging of ROS and/RNS, reduction of oxidative damage/oxidative stress, and therefore, an increased therapeutic benefit in respect of the disease(s), disorder(s) and/or condition(s) associated with oxidative stress. Moreover, based on the ability of the 1H-pyrazole analogue(s) described herein to have the multitude of functions described herein, in embodiments, the 1H-pyrazole analogue(s) described herein can function as a therapeutic for prevention and/or treatment of a disease, condition and/or disorder associated with oxidative stress, such as an ALS therapeutic in vivo.

In other embodiments, the muscle diseases, disorders, and/or conditions comprise muscular dystrophy. In embodiments, the vascular diseases, disorders, and/or conditions comprise cerebral infarction. In other embodiments, the systemic inflammatory diseases, disorders, and/or conditions comprise multiple sclerosis and/or systemic scleroderma. In embodiments, the local inflammatory diseases, disorders, and/or conditions comprise stomatitis.

Other specific diseases, disorders and/or conditions associated with oxidative stress include, for example: (i) metabolic syndrome, including, but not limited to, insulin resistance, obesity, hyperglycemia, dyslipidemia, hypertension and/or diabetes; (ii) cardiovascular diseases, disorders, and/or conditions, including but not limited to, atherosclerosis, hypertension, heart failure, cardiovascular ischemia, and/or myocardial infarction; (iii) autoimmune diseases, disorders, and/or conditions, including but not limited to, rheumatoid arthritis, and/or systemic lupus erythematosus; (iv) inflammatory lung diseases, disorders, and/or conditions, including, but not limited to, chronic obstructive pulmonary disease (COPD), emphysema, and/or asthma; (v) kidney diseases, disorders and/or conditions, including, but not limited to, renal toxicity (drug-induced kidney disease), acute kidney injury (AKI), chronic kidney disease (CKD), diabetic nephropathy, and/or end-stage renal disease (ESRD); (vi) liver diseases, disorders, and/or conditions, including, but not limited to, hepatotoxicity, viral hepatitis, cirrhosis; (vii) digestive diseases, disorders, and/or conditions, including, but not limited to, inflammatory bowel disease (IBD), ulcerative colitis, crohn's disease, gastritis, pancreatitis and/or peptic ulcer; (viii) viral infectious diseases, disorders, and/or conditions, including, but not limited to, blood-borne hepatitis viruses (B, C, and D), human immunodeficiency virus (HIV), influenza A, Epstein-Barr virus, and/or respiratory syncytial virus; (ix) cancer, including, but not limited to, prostate cancer, breast cancer, lung cancer, colorectal cancer, bladder cancer, ovarian cancer, skin cancer, stomach cancer, and/or liver cancer; and (x) sepsis/septic shock. Subjects suffering from any of the aforementioned diseases, disorders and/or conditions associated with oxidative stress may be identified by any, or a combination of, diagnostic or prognostic assay(s) known in the art.

The 1H-Pyrazole analogue(s) described herein may be used in methods for preventing and/or treating a oxidative stress disease, condition and/or disorder. Thus, oxidative stress disease(s), condition(s) and/or disorder(s) may be treatable and/or preventable by administration or delivery of the 1H-pyrazole analogue(s) as described herein. In embodiments, the methods comprise administering the 1H-pyrazole analogue(s) to a subject in need thereof.

The subject referred to herein is, typically, someone who is suffering from one or more oxidative stress disease(s), condition(s) and/or disorder(s), and/or who is at risk of suffering from one or more oxidative disease(s), condition(s) and/or disorder(s). In this way, the uses and methods described herein can be used to prevent the onset of an oxidative stress disease, disorder and/or condition, if for example, the 1H-pyrazole analogue(s) are provided (e.g. administered) to the subject who is at risk of developing the oxidative stress disease, disorder, and/or condition described herein. Alternatively, the uses and methods described herein can be used to treat disease, disorder and/or condition associated with oxidative stress, if for example, the 1H-pyrazole analogue(s) are provided (e.g. administered) to the subject who is suffering from the disease, condition and/or disorder associated with oxidative stress. For purposes of the present disclosure, the subject may have a single disease, condition, and/or disorder associated with oxidative stress, or a constellation of diseases, conditions, and/or disorders associated with oxidative stress, that are to be treated by the uses and methods described herein.

In other embodiments, the 1H-pyrazole analogue(s) described herein can be used in the manufacture of a medicament, and typically the medicament is for the prevention and/or treatment of the diseases, conditions and/or disorders associated with oxidative stress described herein. In typical embodiments, the 1H-pyrazole analogue(s), as a medicament, are for administration to a subject (e.g. mammals, typically humans) in need thereof.

The 1H-pyrazole analogue(s) can be administered to mammals, typically humans. When administered as a pharmaceutical composition, the 1H-pyrazole analogue(s) may be provided in combination with pharmaceutically acceptable carriers or diluents, optionally with pharmaceutically acceptable adjuvants, such as alum. The 1H-pyrazole analogue(s) may, therefore, be suitably formulated into a pharmaceutical composition for administration to human subjects in a biologically compatible form suitable for administration in vivo.

Accordingly, in embodiments, the pharmaceutical composition comprises 1H-pyrazole analogue(s), in admixture with a suitable diluent or carrier. The compositions containing 1H-pyrazole analogue(s) can be prepared by known methods for the preparation of pharmaceutically acceptable compositions which can be administered to subjects, such that an effective quantity of the 1H-pyrazole analogue(s) is combined in a mixture with a pharmaceutically acceptable carrier. Suitable carriers are described, for example, in Remington's Pharmaceutical Sciences (2003-20th edition), in The United States Pharmacopeia: The National Formulary (USP 24 NF19) published in 1999 and in the Handbook of Pharmaceutical Additives (compiled by Michael and Irene Ash, Gower Publishing Limited, Aldershot, England (1995)), the references of which are incorporated by reference in their entirety. On this basis, the composition may include solution(s) of the 1H-pyrazole analogue(s) in association with one or more pharmaceutically acceptable carrier(s) or diluent(s), and contained in buffered solution(s) with a suitable pH and iso-osmotic with the physiological fluids. Solution(s) of 1H-pyrazole analogue(s) can be prepared in water suitably mixed with suitable excipients. Under ordinary conditions of storage and use, these preparations can contain a preservative to prevent the growth of microorganisms. A person skilled in the art would know how to prepare suitable formulations/compositions. In this regard, reference can be made to U.S. Pat. No. 5,843,456, which is incorporated herein by reference.

The 1H-Pyrazole analogue(s) may be administered alone or in combination with other components/ingredients/actives. For example, the 1H-Pyrazole analogue(s) may be administered as a pharmaceutical composition. The described 1H-Pyrazole analogue(s) and/or compositions thereof, may be administered to a subject in a variety of forms depending on the selected route of administration, as will be understood by those skilled in the art. The 1H-Pyrazole analogue(s), and the pharmaceutical composition(s) thereof, may be administered, for example, by oral, parenteral (e.g. intravenous, intraperitoneal, subcutaneous, intramuscular, transepithelial, nasal, intrapulmonary, intrathecal, rectal and topical modes of administration), buccal, sublingual, patch, pump or transdermal administration. In typical embodiments, the 1H-Pyrazole analogue(s) described herein are administered, or are for administration, using oral or intravenous routes of administration.

If the 1H-Pyrazole analogue(s) are administered orally, the selected compound may be administered, for example, in the form of an ingestible powder (e.g. pure powder), tablets or capsules, or as an aqueous solution or suspension. Examples may include: ingestible tablets, buccal tablets, troches, capsules, elixirs, suspensions, syrups, wafers, and the like. For tablet dosage forms, depending on dose, the 1H-Pyrazole analogue(s) may make up from 1 wt % to 99 wt % of the dosage form, more typically from 5 wt % to 60 wt % of the dosage form. Moreover, carriers which are commonly used include lactose and corn starch, and lubricating agents, such as magnesium stearate, calcium stearate, zinc stearate, sodium stearyl fumarate, and mixtures of magnesium stearate with sodium lauryl sulphate, are commonly added. In addition, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl substituted hydroxypropyl cellulose, starch, pregelatinized starch and sodium alginate. Useful diluents include lactose (monohydrate, spray dried monohydrate, anhydrous and the like), mannitol, xylitol, dextrose, sucrose, sorbitol, microcrystalline cellulose, starch and dibasic calcium phosphate dihydrate, and suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Other conventional ingredients include anti-oxidants, colorants, flavoring agents, preservatives and taste masking agents. Tablet blends may be compressed directly or by roller to form tablets. Tablet blends or portions of blends may alternatively be wet, dry, or melt granulated, melt congealed, or extruded before tableting. The final formulation may include one or more layers and may be coated or uncoated; or encapsulated. The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0 8247 6918 X), the disclosure of which is incorporated herein by reference in its entirety.

When aqueous suspensions are prepared for oral use, the active ingredient can be combined with emulsifying and suspending agents. If desired, certain sweetening and/or flavoring agents may be added. The 1H-Pyrazole analogue(s) may be orally administered, for example, with an inert diluent or with an assimilable edible carrier, or it may be enclosed in hard or soft shell gelatin capsules, or it may be compressed into tablets, or it may be incorporated directly into food. For oral therapeutic administration, the 1H-Pyrazole analogue(s) may be incorporated with excipient(s) and used in the form of ingestible tablets, buccal tablets, troches, capsules, elixirs, suspensions, syrups, wafers, and the like.

If the 1H-Pyrazole analogue(s) are administered parenterally, the parenteral administration may be by continuous infusion, bolus, or intermittent bolus and may be over a selected period of time. Suitable examples of devices for parenteral administration include needle (including micro needle) injectors, needle free injectors and infusion techniques. For intramuscular, intraperitoneal, subcutaneous and intravenous use, sterile solutions of the active ingredient are usually prepared, and the pH of the solutions should be suitably adjusted and buffered. For intravenous use, the total concentration of solutes may be controlled in order to render the preparation isotonic. Thus, in embodiments, one or more of the 1H-Pyrazole analogue(s) described herein may be prepared in isotonic medium and administered intravenously.

The pharmaceutical forms suitable for injectable use may include sterile aqueous solutions or dispersion and sterile powders for the extemporaneous preparation of sterile injectable solutions or dispersions. In embodiments, the form is sterile and the fluid is easily syringeable. The preparation of parenteral kits for reconstitution at point-of-care under sterile conditions, for example, by lyophilization, may readily be accomplished using standard pharmaceutical techniques known to those skilled in the art.

1H-Pyrazole analogue(s), including a pharmaceutical composition thereof, for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device (e.g. nebuliser, for example, to create a mist-like dispersion of 1H-Pyrazole analogue(s) such as an aqueous vehicle (e.g. saline)). Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

1H-Pyrazole analogue(s), including a pharmaceutical composition thereof, suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter. In embodiments, a delivery system can be used to deliver the 1H-Pyrazole analogue(s) (e.g. formulations or pharmaceutical compositions). It is understood that the delivery system itself may include a device such as an implantable device.

The 1H-Pyrazole analogue(s) may be combined with soluble macromolecular entities, such as cyclodextrin and suitable analogues thereof or polyethylene glycol containing polymers, in order to improve their solubility, dissolution rate, taste masking, bioavailability and/or stability for use in any of the aforementioned modes of administration. Regardless of the route of administration selected, the 1H-Pyrazole analogue(s), which may be used in a suitable hydrated form, and/or the pharmaceutical compositions thereof, are formulated into pharmaceutically acceptable dosage forms by conventional methods known to those of skill in the art.

Actual dosage levels of the v analogue(s) may be varied so as to obtain an amount which is effective to achieve the desired therapeutic response for a particular patient, composition, and mode of administration. To this regard, the dosage of the 1H-Pyrazole analogue(s) can depend upon the pharmacokinetic and pharmacodynamic properties of the 1H-Pyrazole analogue and its mode and route of administration; the rate of release of the 1H-Pyrazole analogue, the age, sex, health, medical condition, the nature and extent of the symptoms and weight of the recipient, the renal and hepatic function of the patient; the frequency of the treatment and the type of concurrent treatment, if any, and the clearance rate of the 1H-Pyrazole analogue(s) in the subject to be treated and the effect desired. The selected dosage level may also depend on the additional factors including the activity of the particular 1H-Pyrazole analogue(s) and pharmaceutical compositions described herein, the time of administration, the rate of excretion or metabolism of the particular 1H-Pyrazole analogue(s) being employed, the rate and extent of absorption, the duration of the treatment, other drugs that may be administered to the patient, compounds and/or materials used in combination with the particular 1H-Pyrazole analogue(s) employed and like factors well known in the medical arts. One of skill in the art can determine the appropriate dosage based on the above factors.

A physician or veterinarian having ordinary skill in the art can readily determine and prescribe the effective amount of the 1H-Pyrazole analogue(s) or pharmaceutical composition thereof. For example, the physician or veterinarian could start doses of the 1H-Pyrazole analogue(s) employed in the pharmaceutical composition at levels lower than that required in order to achieve the desired therapeutic effect and gradually increase the dosage until the desired effect is achieved. In general, a suitable daily dose of the 1H-Pyrazole analogue(s) will be that amount of the compound which is the lowest dose effective to produce a therapeutic effect. Such an effective dose will generally depend upon the factors described above.

The 1H-Pyrazole analogue(s) may be administered initially in a suitable dosage that may be adjusted as required, depending on the clinical response. For ex vivo treatment of cells over a short period, for example for 30 minutes to 1 hour or longer, higher doses of the 1H-Pyrazole analogue(s) may be used than for long term in vivo therapy.

In some embodiments, the 1H-Pyrazole analogue(s) may be administered in an amount from about 0.001 mg/kg of body weight to about 1000 mg/kg of body weight per day; such as from about 0.01 mg/kg of body weight to about 500 mg/kg of body weight per day; from about 0.01 mg/kg of body weight to about 250 mg/kg of body weight per day; or 0.01 mg/kg of body weight to about 100 mg/kg of body weight per day, and any intermediate ranges or specific amounts, such as from about 0.001 mg/kg, about 0.01 mg/kg, about 0.1 mg/kg, about 1 mg/kg, about 10 mg/kg, about 25 mg/kg, about 50 mg/kg, about 75 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 600 mg/kg, about 700 mg/kg, about 800 mg/kg, about 900 mg/kg, or about 1000 mg/kg, to about 0.001 mg/kg, about 0.01 mg/kg, about 0.1 mg/kg, about 1 mg/kg, about 10 mg/kg, about 25 mg/kg, about 50 mg/kg, about 75 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 600 mg/kg, about 700 mg/kg, about 800 mg/kg, about 900 mg/kg, or about 1000 mg/kg of body weight per day, per hour, per week, or per dose.

As a typical specific example, for the dose per administration of the pharmaceutical composition, in terms of a dose of 1H-Pyrazole analogue(s) for adults, the lower limit is, for example, about 40 mg or about 70 mg, the upper limit is, for example, about 400 mg, about 140 mg, about 120 mg, or about 105 mg, and the range is, for example, from about 40 mg to about 400 mg, preferably from about 40 to about 140 mg, more preferably from about 40 to about 120 mg, and even more preferably from about 40 mg to about 105 mg, and the dose is particularly preferably about 40 mg, about 50 mg, about 60 mg, or about 70 mg, and in particular, preferably about 45 mg or about 55 mg, and most preferably about 50 mg. If intravenous administration is desired, in typical embodiments, the doses will range from about 0.01 to about 10 mg/kg/minute during a constant rate infusion.

If formulated as a fixed dose, such combination products employ the 1H-Pyrazole analogue(s) within the dosage range described above and the other pharmaceutically active agent(s) within its approved dosage range. The 1H-Pyrazole analogue(s) may alternatively be used sequentially with known pharmaceutically acceptable agent(s) when a combination formulation is inappropriate.

Therapeutically effective amounts of the compounds will generally range up to the maximally tolerated dosage, but may vary widely. The precise amounts employed by the attending physician will vary, of course, depending on the 1H-Pyrazole analogue(s), route of administration, physical condition of the patient (e.g. age, weight, and response of the individual patient, as well as the severity of the patient's symptoms) and other factors. The daily dosage may be administered as a single dosage or may be divided into multiple doses, such as two, three, or four times daily, for administration. Alternatively, the doses may be provided on a weekly, biweekly, or monthly basis. In some embodiments, reduced dosages may be used as compared to conventional therapeutic dosages of known agents.

It will be understood that the 1H-Pyrazole analogue(s) may also be combined and/or co-administered with other therapeutic agent(s) that are selected for their particular usefulness against the diseases, conditions and/or disorders associated with oxidative stress described herein. In other words, the 1H-Pyrazole analogue(s) may be combined and/or co-administered with therapeutic agent(s) to treat and/or prevent the diseases, conditions and/or disorders associated with oxidative stress as described herein. As an example of therapeutic agent(s) that may be useful in combination with the 1H-Pyrazole analogue(s) described herein for the treatment and/or prevention of ALS, these therapeutic agents include, but are not limited to, riluzole (Rilutek™), edaravone (Radicava™), mecasermin, baclofen (Lioresal™), diazepam (Valium™), dantrolene (Dantrium™), nonsteroidal anti-inflammatory agents, anticonvulsive medications (e.g., carbamazepine (Tegretol) or phenytoin (Dilantin™)), amitriptyline (Elavil™), nortriptyline (Pamelor™), and Lorazepam (Ativan™). In some embodiments, the additional therapy includes co-administration of elamipretide (a.k.a. SS-31 or Bendavia). In these embodiments, the therapeutic agent(s) administered in combination the 1H-Pyrazole analogue(s) described herein may result in a synergistic therapeutic effect, such that, for example, this combination has a greater than additive effect(s) in the prevention and/or treatment of ALS. Therefore, lower doses of one or more of any individual therapeutic agent may be used, when in combination with the 1H-Pyrazole analogue(s) described herein, in treating or preventing ALS, resulting in increased therapeutic efficacy and decreased side-effects. The combination of 1H-Pyrazole analogue(s) and therapeutic agent(s) described herein may be concurrently or consecutively, in any order. In embodiments, the 1H-Pyrazole analogue(s) and the therapeutic agent(s), act additively or synergistically to prevent and/or treat the oxidative stress disease, condition and/or disorder.

Additional embodiments include:
Embodiment 1. A compound having a structure of Formula I:

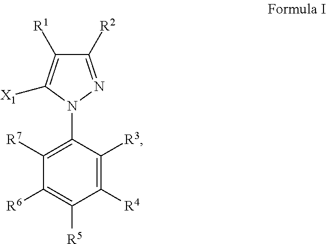

a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof,
   wherein:
   X₁ is selected from —BR⁸R⁹ or —BR¹⁰R¹¹R¹²;
   R¹ to R⁷, R¹⁰, R¹¹, and R¹² are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, —BR¹³R¹⁴, —BR¹⁵R¹⁶R¹⁷, a substituted or unsubstituted carbocyclic group, a substituted or unsubstituted heterocyclic group, a substituted or unsubstituted aromatic group, or a substituted or unsubstituted heteroaromatic group, wherein R¹³ to R¹⁷ are each independently selected from H, a halo group, a hydroxyl group, a cyano group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group or R¹³ and R¹⁴ are taken together to form a substituted or unsubstituted carbocyclic group, or a substituted or unsubstituted heterocyclic group; and
   R⁸ and R⁹ are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, a substituted or unsubstituted heteroaromatic group, or are taken together to form a substituted or unsubstituted carbocyclic group, or a substituted or unsubstituted heterocyclic group.
Embodiment 2. The compound according to embodiment 1, wherein X₁ is —BR⁸R⁹.
Embodiment 3. The compound according to embodiment 1 or 2, wherein R⁸ and R⁹ are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, a substituted or unsubstituted heteroaromatic group.
Embodiment 4. The compound according to any one of embodiments 1 to 3, wherein R⁸ and R⁹ are each independently selected from H, a halo group, a hydroxyl group, a cyano group, a nitro group, a sulfate group, a substituted or unsubstituted alkyl group, a substituted or unsubstituted alkenyl group, a substituted or unsubstituted alkynyl group, a substituted or unsubstituted alkoxy group, —C(O)H, a substituted carbonyl group, a substituted carboxyl group, —C(O)OH, a substituted or unsubstituted carbocyclic group, a substituted or unsubstituted heterocyclic group, a substituted or unsubstituted aromatic group, or a substituted or unsubstituted heteroaromatic group.
Embodiment 5. The compound according to any one of embodiments 1 to 4, wherein R⁸ and R⁹ are each independently selected from H, a halo group, a hydroxyl group, a cyano group, a nitro group, a sulfate group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a substituted or unsubstituted C₁-C₆ cycloalkyl group, a substituted or unsubstituted C₁-C₆ heterocyclic group, a substituted or unsubstituted C₁-C₆ aromatic group, or a substituted or unsubstituted C₁-C₆ heteroaromatic group.
Embodiment 6. The compound according to any one of embodiments 1 to 5, wherein R⁸ and R⁹ are each independently selected from a fluoro group, a chloro group, a bromo group, a hydroxyl group, or alkoxy group.
Embodiment 7. The compound according to embodiment 1 or 2, wherein R⁸ and R⁹ are taken together to form a substituted or unsubstituted carbocyclic group, or a substituted or unsubstituted heterocyclic group.
Embodiment 8. The compound according to any one of embodiments 1, 2 and 7, wherein R⁸ and R⁹ are taken together to form a substituted or unsubstituted a substituted or unsubstituted heterocyclic group.
Embodiment 9. The compound according to any one of embodiments 1, 2, 7 and 8, wherein R⁸ and R⁹ are taken together to form a substituted or unsubstituted —O(C₂-C₈ alkylene)O— ring.
Embodiment 10. The compound according to any one of embodiments 1, 2, and 7 to 9, wherein R⁸ and R⁹ are taken together to form —OCH₂CH₂O—, —OC(CH₃)₂CH₂O—, or —OC(CH₃)₂C(CH₃)₂O—.
Embodiment 11. The compound according to any one of embodiments 1, 2, 7 and 8, wherein R⁸ and R⁹ are taken together to form a substituted or unsubstituted —O(C₁-C₂ alkylene)NH(C₁-C₂ alkylene)O— ring.
Embodiment 12. The compound according to any one of embodiments 1, 2, and 7 to 9, wherein R⁸ and R⁹ are taken together to form —OCH₂CH₂NHCH₂CH₂O—, —OCH₂CH₂N(CH₃)CH₂CH₂O—, —OCH₂C(CH₃)₂NHCH₂CH₂O—, —OCH₂C(CH₃)₂N(CH₃)CH₂CH₂O—, —OCH₂C(CH₃)₂NHC(CH₃)₂CH₂O, —OC(CH₃)₂CH₂N(CH₃)C(CH₃)₂CH₂O—, or —OC(CH₃)₂C(CH₃)₂N(CH₃)C(CH₃)₂C(CH₃)₂O—.
Embodiment 13. The compound according to embodiment 1, wherein X₁ is —BR¹⁰R¹¹R¹²,
Embodiment 14. The compound according to embodiment 1 or 13, wherein R¹⁰, R¹¹ and R¹² are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, a substituted or unsubstituted heteroaromatic group.
Embodiment 15. The compound according to any one of embodiments 1, 13 and 14, wherein R¹⁰, R¹¹ and R¹² are each independently selected from H, a halo group, a hydroxyl group, a cyano group, a nitro group, a sulfate group, a substituted or unsubstituted alkyl group, a substituted or unsubstituted alkenyl group, a substituted or unsubstituted alkynyl group, a substituted or unsubstituted alkoxy group, —C(O)H, a substituted carbonyl group, a substituted carboxyl group, —C(O)OH, a substituted or unsubstituted carbocyclic group, a substituted or unsubstituted heterocyclic group, a substituted or unsubstituted aromatic group, or a substituted or unsubstituted heteroaromatic group.
Embodiment 16. The compound according to any one of embodiments 1 and 13 to 15, wherein R¹⁰, R¹¹ and R¹² are each independently selected from H, a halo group, a hydroxyl group, a cyano group, a nitro group, a sulfate group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a substituted or unsubstituted C₁-C₆ cycloalkyl group, a substituted or unsubstituted C₁-C₆ heterocyclic group, a substituted or unsubstituted C₁-C₆ aromatic group, or a substituted or unsubstituted C₁-C₆ heteroaromatic group.
Embodiment 17. The compound according to any one of embodiments 1 and 13 to 16, wherein R¹⁰, R¹¹ and R¹² are each independently selected from a fluoro group, a chloro group, a bromo group, a hydroxyl group, or an alkoxy group.
Embodiment 18. The compound according to any one of embodiments 1 to 17, wherein R¹ and R² are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group.
Embodiment 19. The compound according to any one of embodiments 1 to 18, wherein R¹ and R² are each independently selected from H, a halo group, a hydroxyl group, a cyano group, a substituted or unsubstituted amino group, a substituted or unsubstituted alkyl group, a substituted or unsubstituted alkenyl group, a substituted or unsubstituted alkynyl group, a substituted or unsubstituted alkoxy group, a substituted or unsubstituted carbocyclic group, a substituted or unsubstituted heterocyclic group, a substituted or unsubstituted aromatic group, or a substituted or unsubstituted heteroaromatic group.
Embodiment 20. The compound according to any one of embodiments 1 to 19, wherein R¹ and R² are each independently selected from H, a halo group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ cycloalkyl group, a substituted or unsubstituted C₁-C₆ heterocyclic group, a substituted or unsubstituted C₁-C₆ aromatic group, or a substituted or unsubstituted C₁-C₆ heteroaromatic group.
Embodiment 21. The compound according to any one of embodiments 1 to 20, wherein R¹ is H or a substituted or unsubstituted alkyl group and R² is selected from H, a halo group, a hydroxyl group, a cyano group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group.
Embodiment 22. The compound according to any one of embodiments 1 to 21, wherein R¹ is H, —CH₃, —CH₂CH₃, or —CH₂CH₂CH₃ and R² is selected from H, F, Cl, CN, —CH₃, —CH₂F, —CHF₂, or —CF₃.
Embodiment 23. The compound according to any one of embodiments 1 to 22, wherein R³ to R⁷ are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, —BR¹³R¹⁴, —BR¹⁵R¹⁶R¹⁷, a substituted or unsubstituted carbocyclic group, a substituted or unsubstituted heterocyclic group, a substituted or unsubstituted aromatic group, or a substituted or unsubstituted heteroaromatic group, wherein R¹³ to R¹⁷ are each independently selected from H, a halo group, a hydroxyl group, a cyano group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group or R¹³ and R¹⁴ are taken together to form a substituted or unsubstituted carbocyclic group, or a substituted or unsubstituted heterocyclic group.
Embodiment 24. The compound according to any one of embodiments 1 to 23, R³ to R⁷ are each independently selected from H, a halo group, a cyano group, a nitro group, a sulfate group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group.
Embodiment 25. The compound according to any one of embodiments 1 to 24, wherein R³ to R⁷ are each independently selected from H, a halo group, a cyano group, a nitro group, a sulfate group, a substituted or unsubstituted alkyl group, a substituted or unsubstituted alkenyl group, a substituted or unsubstituted alkynyl group, a substituted or unsubstituted alkoxy group, —C(O)H, a substituted carbonyl group, a substituted carboxyl group, —C(O)OH, a boronic acid group, a substituted or unsubstituted alkylboronate group, a substituted or unsubstituted carbocyclic group, a substituted or unsubstituted heterocyclic group, a substituted or unsubstituted aromatic group, or a substituted or unsubstituted heteroaromatic group.
Embodiment 26. The compound according to any one of embodiments 1 to 25, wherein R³ to R⁷ are each independently selected from H, a halo group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a boronic acid group, a substituted or unsubstituted C₁-C₆ alkylboronate group, a substituted or unsubstituted C₁-C₆ cycloalkyl group, a substituted or unsubstituted C₁-C₆ heterocyclic group, a substituted or unsubstituted phenyl group, a substituted or unsubstituted alkylheteroaryl group, a substituted or unsubstituted pyridinyl group, or a substituted or unsubstituted pyrrolyl group.
Embodiment 27. The compound according to any one of embodiments 1 to 26, wherein R³, R⁴, R⁶, and R⁷ are each independently selected from H, —CH₃, —CH₂CH₃, or —CH₂CH₂CH₃ and R⁵ is selected from H, F, Cl, CN, —CH₃, —CH₂F, —CHF₂, or —CF₃
Embodiment 28. The compound according to any one of embodiments 1 to 27, wherein the compound is selected from:

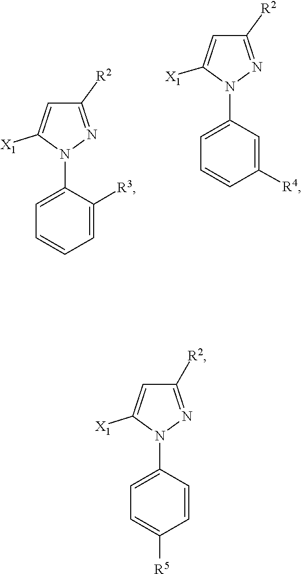

a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof.
Embodiment 29. The compound according to any one of embodiments 1 to 28, wherein the compound is selected from:

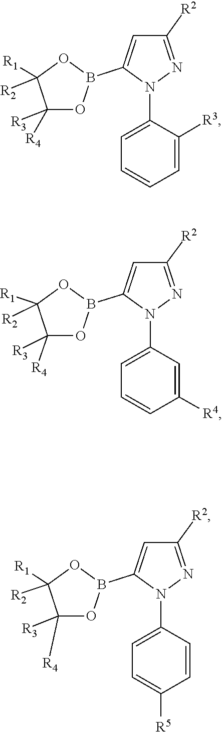

a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof, wherein R₁ to R₄ are each independently selected from any of the groups for R³ to R⁷.
Embodiment 30. The compound according to any one of embodiments 1 to 29, wherein the compound is selected from:

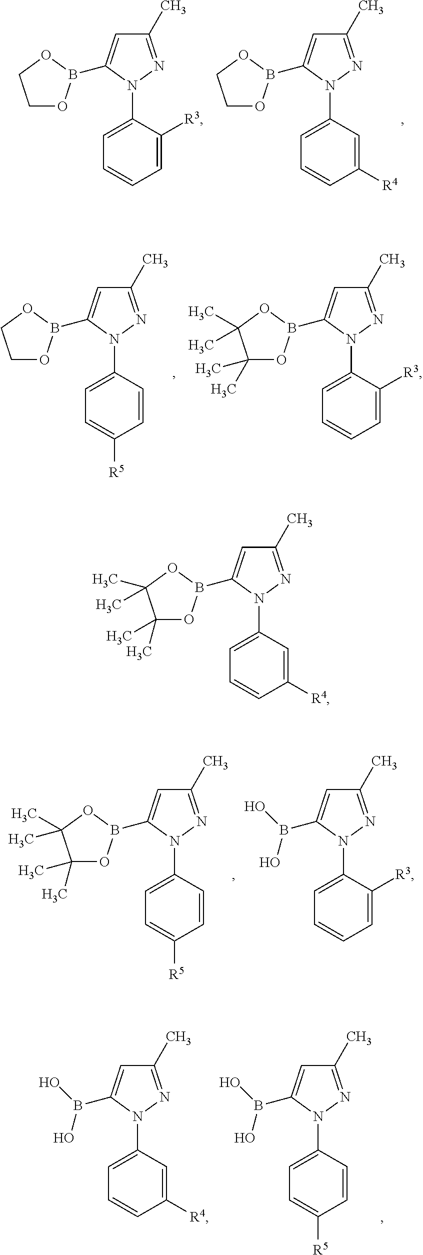

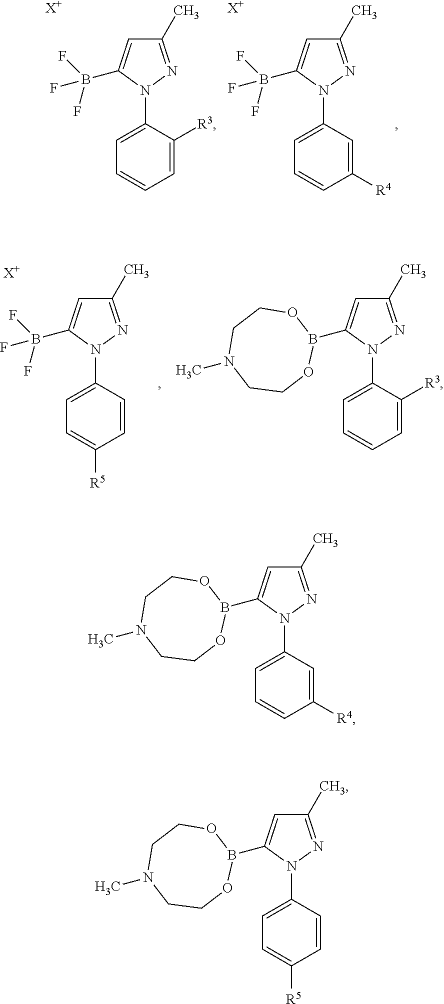

a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof, wherein X⁺ is any suitable counterion.
Embodiment 31. The compound according to any one of embodiments 1 to 30, wherein the compound is selected from:

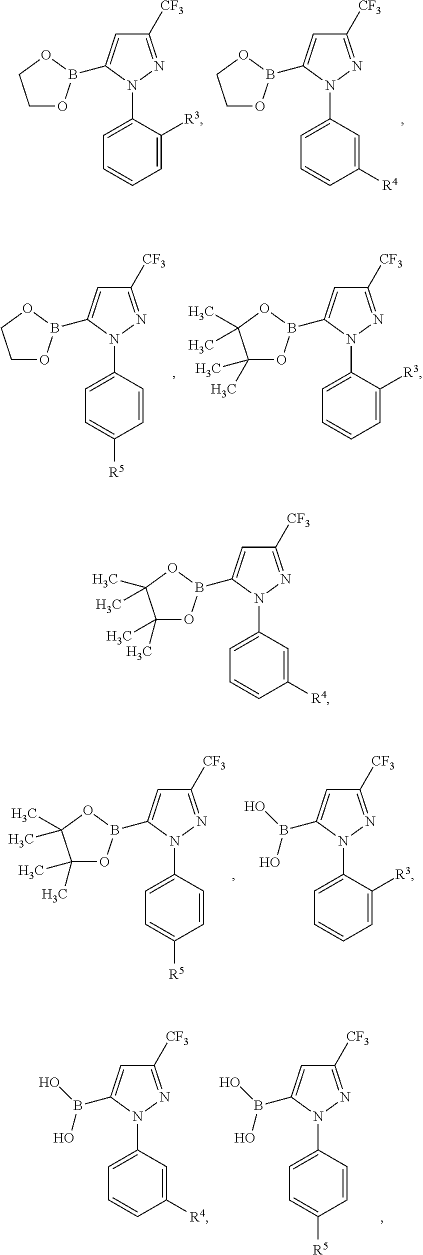

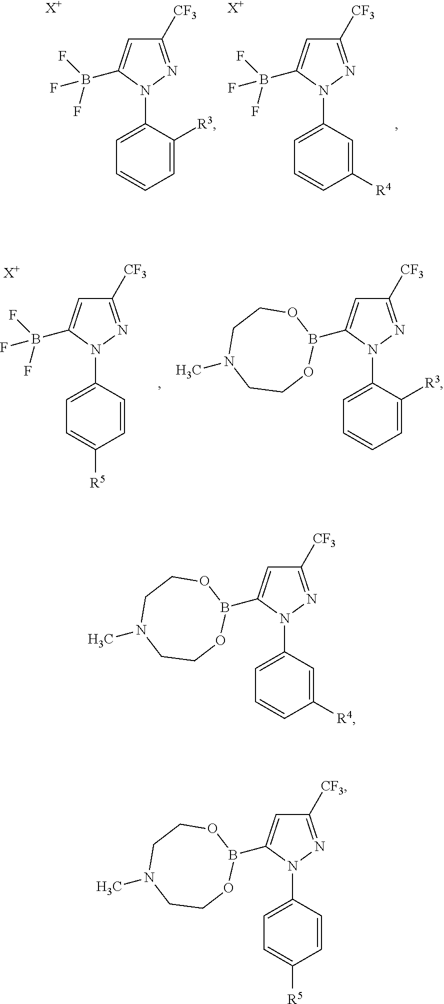

a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof, wherein X⁺ is any suitable counterion.
Embodiment 32. The compound of any one of embodiments 1 to 31, wherein the compound is a racemic mixture.
Embodiment 33 The compound of any one of embodiments 1 to 32, wherein the compound is a scalemic mixture.
Embodiment 34. The compound of any one of embodiments 1 to 33, wherein the compound is a pharmaceutically acceptable salt.
Embodiment 35. The compound according to any one of embodiments 1 to 34, wherein the compound can act as a metal chelator.
Embodiment 36. The compound according to any one of embodiments 1 to 35, wherein the compound can act as an antioxidant.
Embodiment 37. The compound according to any one of embodiments 1 to 36, wherein the compound reduces oxidative stress.
Embodiment 38. The compound according to any one of embodiments 1 to 37, wherein the compound reduces oxidative damage/oxidative stress by scavenging reactive oxygen species (ROS) and/or reactive nitrogen species (RNS).
Embodiment 39. The compound according to any one of embodiments 1 to 38, wherein the compound can reduce reactive oxygen species (ROS) and/or reactive nitrogen species (RNS) in vitro.
Embodiment 40. The compound according to any one of embodiments 1 to 39, wherein the compound can reduce reactive oxygen species (ROS) and/or reactive nitrogen species (RNS) in vivo.
Embodiment 41. The compound according to any one of embodiments 38 to 40, wherein the ROS and/or RNS comprise free radicals and/or oxidants.
Embodiment 42. The compound according to embodiment 41, wherein the free radicals and/or oxidants are selected from hydroxyl radical (HO·), Superoxide radical anion radical (O2·-), nitric oxide (NO·), nitrogen dioxide (NO₂·), peroxyl (ROO·) and lipid peroxyl (LOO·), hydrogen peroxide (H₂O₂), ozone (O₃), singlet oxygen (¹O₂), hypochlorous acid (HOCl), hydroperoxide nucleophile (⁻OOH), nitrous acid (HNO₂), dinitrogen trioxide (N₂O₃), and lipid peroxide (LOOH).
Embodiment 43. The compound of embodiment 41, wherein the free radicals and/or oxidants is hydrogen peroxide (H₂O₂).
Embodiment 44. The compound according to any one of embodiments 1 to 43, wherein the compound can react with hydrogen peroxide in vivo to produce edaravone (EDR).
Embodiment 45. The compound according to any one of embodiments 1 to 44, wherein the compound can react with hydrogen peroxide in vitro to produce edaravone (EDR).
Embodiment 46. The compound according to any one of embodiments 1 to 45, wherein the compound is a prodrug of edaravone (EDR).
Embodiment 47. The compound according to any one of embodiments 1 to 46, wherein the compound is a antioxidant and prodrug of edaravone (EDR).
Embodiment 48. The compound according to any one of embodiments 1 to 47, wherein the compound is an amyotrophic lateral sclerosis (ALS) therapeutic in vivo.
Embodiment 49. The compound according to any one of embodiments 1 to 48, wherein the compound is a therapeutic for prevention and/or treatment of a disease, condition and/or disorder associated with oxidative stress.
Embodiment 50. A pharmaceutical composition comprising the compound according to any one of embodiments 1 to 49.
Embodiment 51. A pharmaceutical composition comprising the compound according to any one of embodiments 1 to 49 and at least one pharmaceutically acceptable carrier and/or diluent.
Embodiment 52. The compound according to any one of embodiments 1 to 49 or the composition according to embodiment 50 or 51 for prevention and/or treatment of a disease, condition and/or disorder associated with oxidative stress.
Embodiment 53. The compound or composition of embodiment 52, wherein the oxidative stress is caused by reactive oxygen species (ROS) and/or reactive nitrogen species (RNS).
Embodiment 54. The compound or composition of embodiment 53, wherein the ROS and/or RNS comprise free radicals and/or oxidants.
Embodiment 55. The compound or composition of any one of embodiments 52 to 54, wherein the free radicals and/or oxidants are selected from hydroxyl radical (HO·), Superoxide radical anion radical (O2·-), nitric oxide (NO·), nitrogen dioxide (NO₂·), peroxyl (ROO·) and lipid peroxyl (LOO·), hydrogen peroxide (H₂O₂), ozone (O₃), singlet oxygen (¹O₂), hypochlorous acid (HOCl), hydroperoxide nucleophile (⁻OOH), nitrous acid (HNO₂), dinitrogen trioxide (N₂O₃), and lipid peroxide (LOOH).
Embodiment 56. The compound or composition of embodiment 54, wherein the free radicals and/or oxidants is hydrogen peroxide (H₂O₂).
Embodiment 57. The compound or composition according to any one of embodiments 52 to 56, wherein the disease, condition and/or disorder associated with oxidative stress is selected from neurodegenerative diseases, disorders and/or conditions, muscle diseases, disorders and/or conditions, vascular diseases, disorders and/or conditions, systemic inflammatory diseases, disorders, and/or conditions, local inflammatory diseases, disorders, and/or conditions, metabolic syndrome, cardiovascular diseases, disorders, and/or conditions, autoimmune diseases, disorders, and/or conditions, inflammatory lung diseases, disorders, and/or conditions, kidney diseases, disorders, and/or conditions, liver diseases, disorders, and/or conditions, digestive diseases, disorders, and/or conditions, aging, disorders and/or conditions, viral infectious diseases, disorders, and/or conditions, cancer, and sepsis/septic shock.
Embodiment 58. The compound or composition according to embodiment 57, wherein the neurodegenerative disease, condition and/or disorder is associated with motor dysfunction.
Embodiment 59. The compound or composition according to embodiment 58, wherein the neurodegenerative disease, condition and/or disorder associated with motor dysfunction is amyotrophic lateral sclerosis (ALS).
Embodiment 60. The compound or composition according to embodiment 59, wherein the ALS is familial ALS.
Embodiment 61. The compound or composition according to embodiment 59, wherein the ALS is sporadic ALS.
Embodiment 62. The compound or composition according to any one of embodiments 59 to 61, wherein the ALS is caused by a mutation in the superoxide dismutase 1 (SOD1) gene or TARDBP gene.
Embodiment 63. The compound or composition according to any one of embodiments 59 to 62, wherein the compound or composition delays the onset of ALS.
Embodiment 64. The compound or composition according to any one of embodiments 52 to 63, wherein the compound or composition has an improved therapeutic index for ALS as compared to edaravone (EDR).
Embodiment 65. The compound or composition according to embodiment 64, wherein the therapeutic index is a measure of one or more of the following symptoms muscle weakness, muscle wasting (atrophy), weight loss (cachexia), muscle fasciculations, muscle spasticity, slowness of movement, poor balance, incoordination, alterations in vocal quality, dysarthria, dysphagia, incomplete eye closure, drooling, pseudobulbar affect, and/or premature death.
Embodiment 66. The compound or composition according to embodiment 64 or 65, wherein the improved therapeutic index is measured by increased survival/lifespan in the subject receiving the compound or composition as compared to the subject receiving edaravone (EDR).
Embodiment 67. The compound or composition according to any one of embodiments 64 to 66, wherein the improved therapeutic index is measured by increased motor function in the subject receiving the compound or composition as compared to a subject receiving edaravone (EDR).
Embodiment 68. The compound or composition according to any one of embodiments 64 to 67, wherein the improved therapeutic index is measured by a lower percentage of weight loss (cachexia), in the subject receiving the compound or composition as compared to a subject receiving edaravone (EDR).
Embodiment 69. The compound or composition according to any one of embodiments 52 to 68, wherein the compound or composition has an improved pharmacokinetics as compared to edaravone (EDR).
Embodiment 70. The compound or composition according to embodiment 69, wherein the improved pharmacokinetics comprises increased bioavailability of the compound or composition as compared to edaravone (EDR).
Embodiment 71. A method for preventing and/or treating a disease, condition and/or disorder associated with oxidative stress, comprising administering to a mammal a therapeutically effective amount of the compound according to any one of embodiments 1 to 49 or the composition according to embodiment 50 or 51.
Embodiment 72. The method according to embodiment 71, wherein the oxidative stress is caused by reactive oxygen species (ROS) and/or reactive nitrogen species (RNS).
Embodiment 73. The method according to embodiment 72, wherein the ROS and/or RNS comprise free radicals and/or oxidants.
Embodiment 74. The method according to embodiment 73, wherein the free radicals and/or oxidants are selected from hydroxyl radical (HO·), Superoxide radical anion radical (O2·-), nitric oxide (NO·), nitrogen dioxide (NO₂·), peroxyl (ROO·) and lipid peroxyl (LOO·), hydrogen peroxide (H₂O₂), ozone (O₃), singlet oxygen (¹O₂), hypochlorous acid (HOCl), hydroperoxide nucleophile (⁻OOH), nitrous acid (HNO₂), dinitrogen trioxide (N₂O₃), and lipid peroxide (LOOH)
Embodiment 75. The method of embodiment 73, wherein the free radicals and/or oxidants is hydrogen peroxide (H₂O₂).
Embodiment 76. The method according to any one of embodiments 71 to 75, wherein the disease, condition and/or disorder associated with oxidative stress is selected from neurodegenerative diseases, disorders and/or conditions, muscle diseases, disorders and/or conditions, vascular diseases, disorders and/or conditions, systemic inflammatory diseases, disorders, and/or conditions, local inflammatory diseases, disorders, and/or conditions, metabolic syndrome, cardiovascular diseases, disorders, and/or conditions, autoimmune diseases, disorders, and/or conditions, inflammatory lung diseases, disorders, and/or conditions, kidney diseases, disorders, and/or conditions, liver diseases, disorders, and/or conditions, digestive diseases, disorders, and/or conditions, aging, disorders and/or conditions, viral infectious diseases, disorders, and/or conditions, cancer, and sepsis/septic shock.
Embodiment 77. The method according to embodiment 76, wherein the neurodegenerative disease, condition and/or disorder is associated with motor dysfunction.
Embodiment 78. The method according to embodiment 77, wherein the neurodegenerative disease, condition and/or disorder associated with motor dysfunction is amyotrophic lateral sclerosis (ALS).
Embodiment 79. The method according to embodiment 78, wherein the ALS is familial ALS.
Embodiment 80. The method according to embodiment 78, wherein the ALS is sporadic ALS.
Embodiment 81. The method according to any one of embodiments 78 to 80, wherein the ALS is caused by a mutation in the superoxide dismutase 1 (SOD1) gene or TARDBP gene.
Embodiment 82. The method according to any one of embodiments 78 to 81, wherein the compound or composition delays the onset of ALS.
Embodiment 83. The method according to any one of embodiments 71 to 82, wherein the compound or composition has an improved therapeutic index for ALS as compared to edaravone (EDR).
Embodiment 84. The method according to embodiment 83, wherein the therapeutic index is a measure of one or more of the following symptoms muscle weakness, muscle wasting (atrophy), weight loss (cachexia), muscle fasciculations, muscle spasticity, slowness of movement, poor balance, incoordination, alterations in vocal quality, dysarthria, dysphagia, incomplete eye closure, drooling, pseudobulbar affect, and/or premature death.
Embodiment 85. The method according to embodiment 83 or 84, wherein the improved therapeutic index is measured by increased survival/lifespan in the subject receiving the compound or composition as compared to the subject receiving edaravone (EDR).
Embodiment 86. The method according to any one of embodiments 83 to 85, wherein the improved therapeutic index is measured by increased motor function in the subject receiving the compound or composition as compared to a subject receiving edaravone (EDR).
Embodiment 87. The method according to any one of embodiments 83 to 86, wherein the improved therapeutic index is measured by a lower percentage of weight loss (cachexia), in the subject receiving the compound or composition as compared to a subject receiving edaravone (EDR).
Embodiment 88. The method according to any one of embodiments 71 to 87, wherein the compound or composition has an improved pharmacokinetics as compared to edaravone (EDR).
Embodiment 89. The method according to embodiment 88, wherein the improved pharmacokinetics comprises increased bioavailability of the compound or composition as compared to edaravone (EDR).
Embodiment 90. The method according to any one of embodiments 71 to 89, wherein the mammal is a human.
Embodiment 91. The method according to any one of embodiments 71 to 90, wherein the compound or composition is administered orally and/or intravenously.
Embodiment 92. Use of a therapeutically effective amount of the compound according to any one of embodiments 1 to 49 or the composition according to embodiment 50 or 51 for preventing and/or treating a disease, condition, and/or disorder associated with oxidative stress.
Embodiment 93. The use according to embodiment 92, wherein the oxidative stress is caused by reactive oxygen species (ROS) and/or reactive nitrogen species (RNS).
Embodiment 94. The use according to embodiment 93, wherein the ROS and/or RNS comprise free radicals and/or oxidants.
Embodiment 95. The use according to embodiment 94, wherein the free radicals and/or oxidants are selected from hydroxyl radical (HO·), Superoxide radical anion radical (O2·-), nitric oxide (NO·), nitrogen dioxide (NO₂·), peroxyl (ROO·) and lipid peroxyl (LOO·), hydrogen peroxide (H₂O₂), ozone (O₃), singlet oxygen (¹O₂), hypochlorous acid (HOCl), hydroperoxide nucleophile (⁻OOH), nitrous acid (HNO₂), dinitrogen trioxide (N₂O₃), and lipid peroxide (LOOH).
Embodiment 96. The use of embodiment 94, wherein the free radicals and/or oxidants is hydrogen peroxide (H₂O₂).
Embodiment 97. The use according to any one of embodiments 92 to 96, wherein the disease, condition and/or disorder associated with oxidative stress is selected from neurodegenerative diseases, disorders and/or conditions, muscle diseases, disorders and/or conditions, vascular diseases, disorders and/or conditions, systemic inflammatory diseases, disorders, and/or conditions, local inflammatory diseases, disorders, and/or conditions, metabolic syndrome, cardiovascular diseases, disorders, and/or conditions, autoimmune diseases, disorders, and/or conditions, inflammatory lung diseases, disorders, and/or conditions, kidney diseases, disorders, and/or conditions, liver diseases, disorders, and/or conditions, digestive diseases, disorders, and/or conditions, aging, disorders and/or conditions, viral infectious diseases, disorders, and/or conditions, cancer, and sepsis/septic shock.
Embodiment 98. The use according to embodiment 97, wherein the neurodegenerative disease, condition and/or disorder is associated with motor dysfunction.
Embodiment 99. The use according to embodiment 98, wherein the neurodegenerative disease, condition and/or disorder associated with motor dysfunction is amyotrophic lateral sclerosis (ALS).
Embodiment 100. The use according to embodiment 99, wherein the ALS is familial ALS.
Embodiment 101. The use according to embodiment 99, wherein the ALS is sporadic ALS.
Embodiment 102. The use according to any one of embodiments 99 to 101, wherein the ALS is caused by a mutation in the superoxide dismutase 1 (SOD1) gene or TARDBP gene.
Embodiment 103. The use according to any one of embodiments 99 to 102, wherein the compound or composition delays the onset of ALS.
Embodiment 104. The use according to any one of embodiments 92 to 103, wherein the compound or composition has an improved therapeutic index for ALS as compared to edaravone (EDR).
Embodiment 105. The use according to embodiment 104, wherein the therapeutic index is a measure of one or more of the following symptoms muscle weakness, muscle wasting (atrophy), weight loss (cachexia), muscle fasciculations, muscle spasticity, slowness of movement, poor balance, incoordination, alterations in vocal quality, dysarthria, dysphagia, incomplete eye closure, drooling, pseudobulbar affect, and/or premature death.
Embodiment 106. The use according to embodiment 104 or 105, wherein the improved therapeutic index is measured by increased survival/lifespan in the subject receiving the compound or composition as compared to the subject receiving edaravone (EDR).
Embodiment 107. The use according to any one of embodiments 104 to 106, wherein the improved therapeutic index is measured by increased motor function in the subject receiving the compound or composition as compared to a subject receiving edaravone (EDR).
Embodiment 108. The use according to any one of embodiments 104 to 107, wherein the improved therapeutic index is measured by a lower percentage of weight loss (cachexia), in the subject receiving the compound or composition as compared to a subject receiving edaravone (EDR).
Embodiment 109. The use according to any one of embodiments 92 to 108, wherein the compound or composition has an improved pharmacokinetics as compared to edaravone (EDR).
Embodiment 110. The use according to embodiment 109, wherein the improved pharmacokinetics comprises increased bioavailability of the compound or composition as compared to edaravone (EDR).
Embodiment 111. The use according to any one of embodiments 92 to 110, wherein the mammal is a human.
Embodiment 112. The use according to any one of embodiments 92 to 111, wherein the compound or composition is administered orally and/or intravenously.

The above disclosure generally describes the present invention. A more complete understanding can be obtained by reference to the following specific examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, specifically point out the typical aspects, and are not to be construed as limiting in any way to the disclosure.

### EXAMPLES

### Example 1 Synthesis, Characterization, and Development of Boron-Based EDR Analogues

B⁵-EDR analogues can serve as prodrugs via oxidative transformations.

Methods: B⁵-EDR analogues were synthesized and the in vitro transformation of the B⁵-EDR analogues, as prodrugs into EDR, was assessed.

Compounds Synthesized are as Follows:

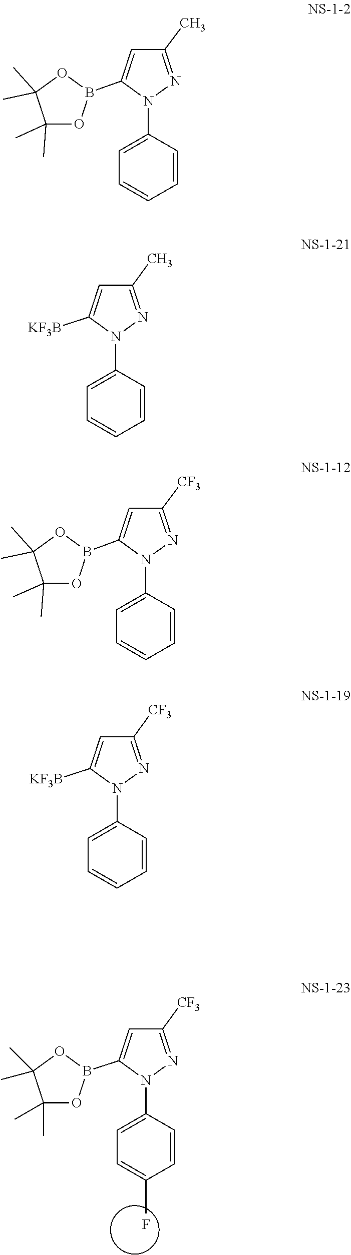

### EXPERIMENTAL

General: ¹H and ¹³C nuclear magnetic resonance (NMR) spectra were recorded on a Bruker 400 MHz and 300 MHz spectrometer, Billerica, MA, USA, using DMSO-d₆ (CAS-2206-27-1), Acetone-d₆ (CAS-666-52-4), Chloroform-d₆ (CAS-865-49-6), Methanol-d₆ (CAS-811-98-3), Acros organics, Switzerland as solvent with tetramethylsilane (TMS) as an internal standard. n-Butyllithium, 2.5M solution in hexanes, Acros organics (CAS-109-72-8) product of Germany; 2-Isopropoxy-4,4,5,5-tetramethyl-1,3,2-dioxaborolane (CAS-61676-62-8) purchased from Sigma-Aldrich, St. Louis, MO, USA; 3-Methyl-1-phenyl-1H-pyrazole (Number: AK139802) was purchased from Ark Pharm, Inc., Arlington Heights, IL, USA; 3-(trifluoromethyl)-1-phenyl-1H-pyrazole (CAS-99498-65-4) was purchased from AK Scientific Ahern Ave, city CA, USA; Copper (I) Oxide (CAS-1317-39-1) was purchased from Sigma-Aldrich, St. Louis, MO, USA; Potassium hydrogen fluoride (CAS-7789-29-9) was purchased from Sigma-Aldrich, St. Louis, MO, USA. The reactions were monitored by TLC (Sigma, Silica gel 60 F₂₅₄). The crude reaction mixture was purified with silica gel column chromatography on a CombiFlash® Rf 200 purification system, Teledyne Isco, USA. Organic solvents were ordered from BDH, VWR Analytical unless specified otherwise. All chemicals were used without further purification unless otherwise indicated.

Scheme I for Synthesis of N-Arylated Pyrazole Boronic Acid Pinacol Ester from the Commercially Available N-Arylated Substituted Pyrazole Starting Material Via Two Step Synthetic Procedure In Situ.

The synthetic route involves synthesis of N-arylated pyrazole boronic acid pinacol ester from the commercially available N-arylated substituted pyrazole starting material via two step synthetic procedure in situ. The first step involves lithiation at C-5 position of N-arylated substituted pyrazole with N-butyl lithium (n-BuLi), by directed ortho metalation (DOM) mechanism. The second step involves electrophilic substitution of Lithium at C-5 position with isopropoxy 4,4,5,5-tetramethyl-1,3,2-dioxaborolane (PINBOP). This is followed by acidic workup which yielded N-arylated substituted pyrazole boronic acid pinacol ester as our first proposed EDR (B⁵-EDR) prodrugs.

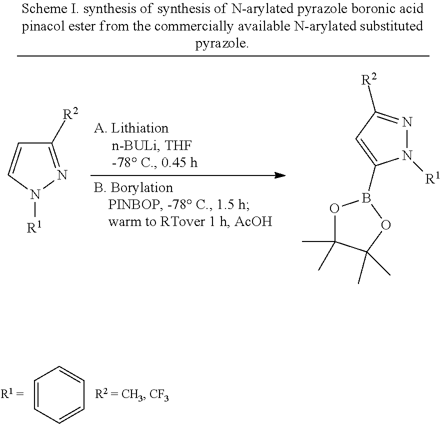

Representative Experimental Procedure for Synthesis of 3-Methyl-5-(4,4,5,5-Tetramethyl-1,3,2-Dioxaborolan-2-yl)-1-Phenyl1H-Pyrazole was Carried Out According to the Below Reported Procedure with Slight Modifications Scheme I [46].

n-Butyllithium (2.5 M in hexane, 1.5 cm³, 3.793 mmol) was added dropwise to a solution of N-arylated substituted pyrazole (500 mg, 0.471 cm³, 3.161 mmol) in anhydrous THF (22 cm³) at −78° C. under argon. The reaction mixture was stirred for 45 min at −78° C. 2-Isopropoxy-4,4,5,5-tetramethyl1,3,2-dioxaborolane (646.93 mg, 709.5 μl, 3.477 mmol) was added dropwise to the reaction mixture at −78° C. and the mixture was stirred for 1.5 h. The mixture was warmed to room temperature over 1 h and glacial acetic acid (208.79 mg, 199 μl, 3.477 mmol) was added. The mixture was filtered through a celite pad, which was washed with EtOAc (100 cm³). The organic solvent was removed in vacuo to afford a crude product. The expected product confirmation was done by TLC (20% EtOAc/Hex). The crude product was then purified with silica gel column chromatography on a CombiFlash® Rf 200 purification system, Teledyne Isco, USA, with ethyl acetate and hexane from 0% to 10%. Residual solvent was evaporated under vacuum to give the product as a light brown crystalline solid (815 mg, 90%).

Compound (NS-1-2) was synthesized by following this reaction procedure. The structure was characterized by ¹H and ¹³C NMR.

3-methyl-5-(4,4,5,5-tetramethyl-1,3,2-dioxaborolan-2-yl)-1-phenyl1H-pyrazole (NS-1-2): Prodrug of Edaravone. Crystalline solid (815 mg, 90%); 1H NMR (400 MHz, CDCl₃-d₆) δ 7.52-7.49 (2H, m, J=12 Hz), δ 7.40-7.36 (2H, m, J=16 Hz), 7.33-7.29 (1H, m, J=16 Hz), δ 6.66 (1H, S), δ 2.35 (3H, S) δ 1.26 (12H, S); 13C NMR (300 MHz, DMSO-d6) δ 147.4, 139.4, 127.0, 125.7, 122.4, 115.9, 82.7, 22.9, 11.4 (C ipso to B not observed).

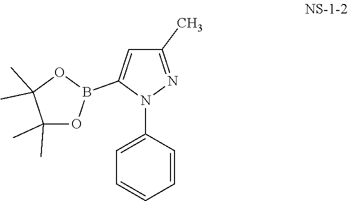

Representative Experimental Procedure for Synthesis of 3-Trifluoromethyl-5-(4,4,5,5-Tetramethyl-1,3,2-Dioxaborolan-2-yl)-1-Phenyl1H-Pyrazole was Carried Out According to the Below Reported Procedure with Slight Modifications Scheme I [46].

n-Butyllithium (2.5 M in hexane, 1.4 cm³, 3.5 mmol, 1 equiv) was added dropwise to a solution of N-arylated substituted pyrazole (617 mg, 0.61 cm³, 2.9 mmol) in anhydrous THF (20 cm³) at −78° C. under argon. The reaction mixture was stirred for 45 min at −78° C. 2-Isopropoxy-4,4,5,5-tetramethyl1,3,2-dioxaborolane (620 μl, 3.1 mmol, 1 equiv) was added dropwise to the reaction mixture at −78° C. and the mixture was stirred for 1.5 h. The mixture was warmed to room temperature over 1 h and glacial acetic acid (180 μl, 3.2 mmol) was added. The mixture was filtered through a celite pad, which was washed with EtOAc (100 cm³). The organic solvent was removed in vacuo to afford a crude product. The expected product confirmation was done by TLC (5% EtOAc/Hex). The crude product was then purified with silica gel column chromatography on a CombiFlash® Rf 200 purification system, Teledyne Isco, USA, with ethyl acetate and hexane from 0% to 10%. Residual solvent was evaporated under vacuum to give the product as a light brown crystalline solid (815 mg, 90%).

Compound (NS-1-12) was synthesized by following this reaction procedure. The structure was characterized by ¹H and ¹³C NMR.

3-triflouromethyl-5-(4,4,5,5-tetramethyl-1,3,2-dioxaborolan-2-yl)-1-phenyl1H-pyrazole (NS-1-12): Crystalline solid (902 mg, 91.9%); 1H NMR (400 MHz, CDCL3-d6) δ 7.55-7.52 (2H, m, J=12 Hz), δ 7.45-7.41 (3H, m, J=16 Hz), 7.11 (1H, S), δ 1.26 (12H, S); 13C NMR (300 MHz, DMSO-d6) δ 142.6, 142.1, 140.4, 129.3, 129.2, 125.3, 115.2, 85.1, 24.7 (C ipso to B not observed).

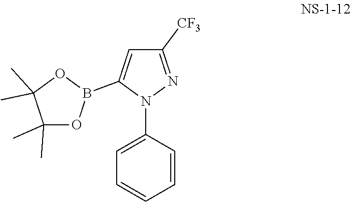 [SMILES: CC1(C)OB(c2cc(C(F)(F)F)nn2-c2ccccc2)OC1(C)C]

Scheme II for Synthesis of N-Arylated Pyrazole Potassium Trifluoroborates from N-Arylated Substituted Pyrazole Boronic Acid Pinacol Ester.

The synthetic route involved synthesis of N-arylated pyrazole potassium trifluoroborates from N-arylated substituted pyrazole boronic acid pinacol ester. It is a three-step synthesis which involves lithiation in first step, borylation in second step to get N-arylated pyrazole boronic acid pinacol ester followed by conversion of the pinacolyl boronate to the corresponding trifluoro borate with aqueous potassium hydrogen fluoride [47].

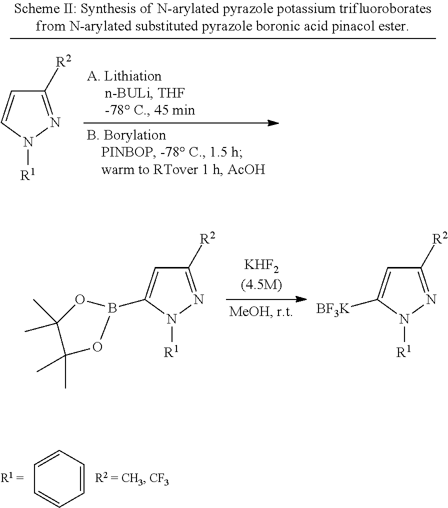

Representative Procedure for Synthesis of N-Arylated Pyrazole Potassium Trifluoroborates from N-Arylated Substituted Pyrazole Boronic Acid Pinacol Ester was Carried Out According to the Below Reported Procedure with Slight Modifications [47].

To a stirred solution of boronic ester (0.5 mmol) in methanol (3 mL) was added KHF₂ (0.5 mL of 4.5M saturated aqueous solution, 0.5 mmol, 2.25 equiv, 1.125-fold excess) dropwise. The resulting mixture was stirred at room temperature and the progress of the reaction was monitored by TLC after every 15 minutes. The reaction was completed with new spot in the TLC after 1 Hr for and 2.5 Hr for. The resulting crude reaction mixture was filtered using 11 cm whatman's filter paper and the filter paper was washed thoroughly with hot acetone to filter all the product from the crude reaction mixture. The residue left in the filter paper is white amorphous solid, which was dissolved in ethyl acetate and TLC was observed under UV. There was no spot corresponding to the product. The filtrate was concentrated in vacuo to remove all the volatile material. The resulting waxy syrup was re-dissolved in 50% aq MeOH (4 mL) and all volatile materials were evaporated again on a rotary evaporator (5-1 mbar/45-50° C.; undesirable bumping of the mixture can be significantly minimized by adjusting the rotation speed). This Evaporation-dissolution cycles were repeated until ¹H NMR analysis of an aliquot of the reaction mixture showed less than 1 mol % of pinacol. The Evaporation-dissolution cycles were optimized for the synthesis of prodrugs. The optimized cycled were 10 for synthesis of prodrug. The final concentrated residue obtained as an amorphous solid, which was finally dried over desiccator overnight to give the product as a white amorphous solid (815 mg, 90%). Some minor loss of product is mainly associated with evaporation/drying/substance transfer manipulations. All compounds (NS-1-21, NS-1-19) were synthesized by following this reaction procedure. Their structures were characterized by ¹H and ¹³C NMR.

Potassium trifluoro(3-methyl-1phenyl-1H-pyrazol-5-yl)borate (NS-1-21): Prodrug of Edaravone.

Amorphous solid (125.2 mg, 94.8%); 1H NMR (400 MHz, acetone-d6) δ 7.27-7.20 (2H, m, J=16 Hz), δ 6.54-6.50 (2H, m, J=16 Hz), 6.36-6.31 (1H, m, J=20 Hz), δ 5.35 (1H, S), δ 1.42 (3H, S); 13C NMR (300 MHz, MeOD-d6) δ 127.8, 125.7, 123.9, 111.3, 74.4, 23.6, 11.5 (C ipso to B not observed).

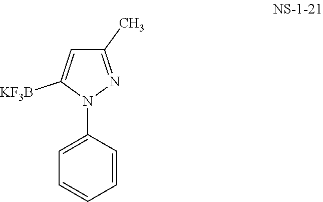

Potassium trifluoro(3-triflouromethyl-1phenyl-1H-pyrazol-5-yl)borate (NS-1-19):

Amorphous solid (143.5 mg, 90.2%); 1H NMR (400 MHz, acetone-d6) δ 7.27-7.20 (2H, m, J=28 Hz), δ 6.67-6.63 (2H, m, J=16 Hz), 6.56-6.52 (1H, m, J=16 Hz), δ 5.86 (1H, S); 13C NMR (300 MHz, acetone-d6) δ 129.6, 127.9, 127.6, 126.1, 123.5, 119.4, 28.9 (C ipso to B not observed).

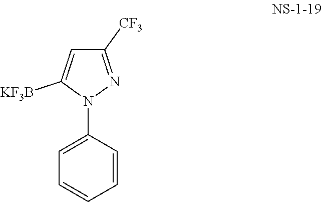

Scheme III Synthesis of 4-Fluoro-N-Arylated Pyrazole Boronic Acid Pinacol Ester from 4-Fluoro-N-Arylated Substituted Pyrazole Starting Material Via Two Step Synthetic Procedure In Situ.

The synthetic route involved synthesis of 4-fluoro-N-arylated pyrazole boronic acid pinacol ester from 4-fluoro-N-arylated substituted pyrazole starting material via two step synthetic procedure in situ. The first step involves N-arylation of pyrazoles with aryl boronic acids using heterogeneous Copper (I) oxide in methanol at room temperature under base free conditions. The second step involves lithiation at C-5 position of N-arylated substituted pyrazole with N-butyl lithium (n-BuLi), by directed ortho metalation (DOM) mechanism. The second step involves electrophilic substitution of Lithium at C-5 position with isopropoxy 4,4,5,5-tetramethyl-1,3,2-dioxaborolane (PINBOP). This is followed by acidic workup which yielded 4-fluoro-N-arylated pyrazole boronic acid pinacol ester [48].

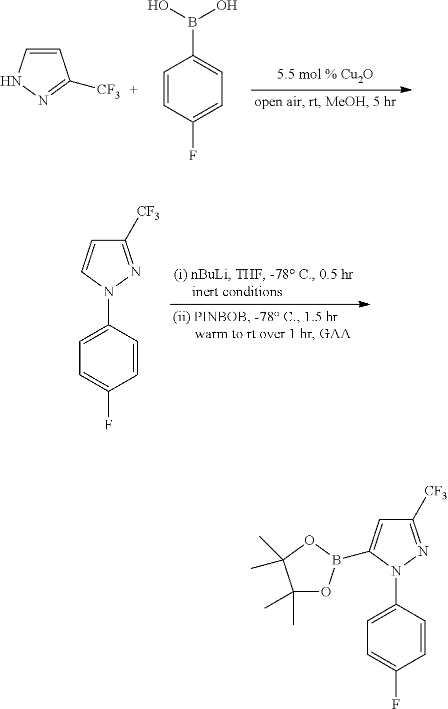 [SMILES: FC(F)(F)c1cc[nH]n1 ; OB(O)c1ccc(F)cc1 ; Fc1ccc(-n2ccc(C(F)(F)F)n2)cc1 ; CC1(C)OB(c2cc(C(F)(F)F)nn2-c2ccc(F)cc2)OC1(C)C]

Figure III: Synthesis of 4-fluoro-N-arylated pyrazole boronic acid pinacol ester from 4-fluoro-N-arylated substituted pyrazole starting material.
Representative experimental procedure for synthesis of 3-trifluoro-methyl-5-(4,4,5,5-tetramethyl-1,3,2-dioxaborolan-2-yl)-4-Fluoro-1-phenyl1H-pyrazole was carried out according to the below reported procedure with slight modifications [46, 48].

The first step involvedN-Arylation of pyrazoles with Arylboronic Acid at Ambient Conditions. 1 mol % Cu₂O (10 mole, 1.02 eq.) was added to a mixture of pyrazole (10 mmol, 1 equiv) and arylboronic acid (10 mmol, 1.2 eqiv) in MeOH (3 ml/mol) at r.t., and the mixture was stirred for 5 h under an atmosphere of air. The progress of the reaction was monitored by TLC and on completion of the reaction, the crude reaction mixture was concentrated under reduced pressure to give the crude product. The crude product was then purified with silica gel column chromatography on a CombiFlash® Rf 200 purification system, Teledyne Isco, USA, with ethyl acetate and hexane from 0%-2%. Residual solvent was evaporated under vacuum to give the light green waxy syrup, which when kept in ice bath for 20 minutes, appeared as green crystals to give the product as a light green crystalline form (2096 mg, 90.8%). Compound (NS-1-22) is synthesized by following this reaction procedure. Their structures were characterized by ¹H and ¹³C NMR.

In the second step, n-Butyllithium (2.5 M in hexane, 1.4 cm³, 3.5 mmol, 1 equiv) was added dropwise to a solution of N-arylated substituted pyrazole (667 mg, 2.9 mmol, 1 equiv) in anhydrous THF (20 cm³) at −78° C. under argon. The reaction mixture was stirred for 45 min at −78° C. 2-Isopropoxy-4,4,5,5-tetramethyl1,3,2-dioxaborolane (620 μl, 3.1 mmol, 1 equiv) was added dropwise to the reaction mixture at −78° C. and the mixture was stirred for 1.5 h. The mixture was warmed to room temperature over 1 h and glacial acetic acid (180 μl, 3.2 mmol) was added. The mixture was filtered through a celite pad, which was washed with EtOAc (100 cm³). The organic solvent was removed in vacuo to afford a crude product. The expected product confirmation was done by TLC (20% EtOAc/Hex). The crude product was then purified with silica gel column chromatography on a CombiFlash® Rf 200 purification system, Teledyne Isco, USA, with ethyl acetate and hexane from 0% to 5%. Residual solvent was evaporated under vacuum to give the crude product in the form of waxy syrup. The syrup was dissolved in pure hexane and kept in −80° C. overnight. After 12 h hexane was removed by rotavapor, and the product was isolated as crystals (556 mg, 53.8%) under room temperature. Compound (NS-1-23) is synthesized by following this reaction procedure. Their structures were characterized by ¹H and ¹³C NMR.

3-triflouromethyl-5-(4,4,5,5-tetramethyl-1,3,2-dioxaborolan-2-yl)-4-fluoro-1-phenyl1H-pyrazole (NS-1-23): Crystalline solid (556 mg, 53.8%); 1H NMR (300 MHz, DMSO-d6) δ 7.66-7.61 (2H, m, J=15 Hz), δ 7.36-7.22 (2H, m, J=42 Hz), 7.22 (1H, S), δ 1.20 (12H, S); 13C NMR (300 MHz, DMSO-d6) δ 142.6, 136.9, 127.8, 127.7, 116.1, 115.8, 115.2, 85.0, 24.8 (C ipso to B not observed); F19 NMR (300 MHz, DMSO-d6) δ −60.32.

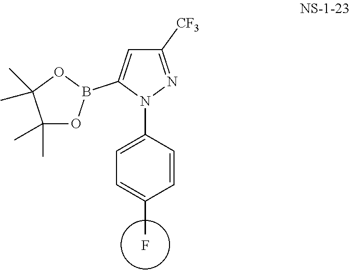

The C-5 position of the pyrazole scaffold of EDR was modified using a ‘boron-prodrug approach’, FIG. 3.

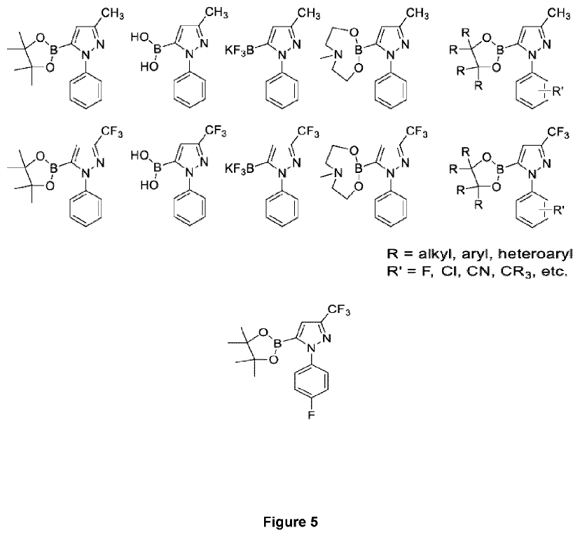

Results: As shown in FIGS. 4A-B, these analogues undergo near quantitative conversion to EDR using hydrogen peroxide in vitro. In fact, there was successful conversion of B⁵-EDR into EDR using H₂O₂ in 95% yield. As such, the synthesized B⁵-EDR compounds can serve as EDR prodrugs under oxidative conditions. In addition, FIG. 5 shows a variety of B⁵-EDR analogues that were synthesized.

### Example 2 Test Boron-Based EDR Analogue Ability to be Redox Regulators

B⁵-EDR analogues can serve as independent redox regulators via the antioxidant effects of boron and serve as neurocytoprotective agents, in a similar manner to EDR, including a similar neurotoxicity/cell viability profile to EDR.

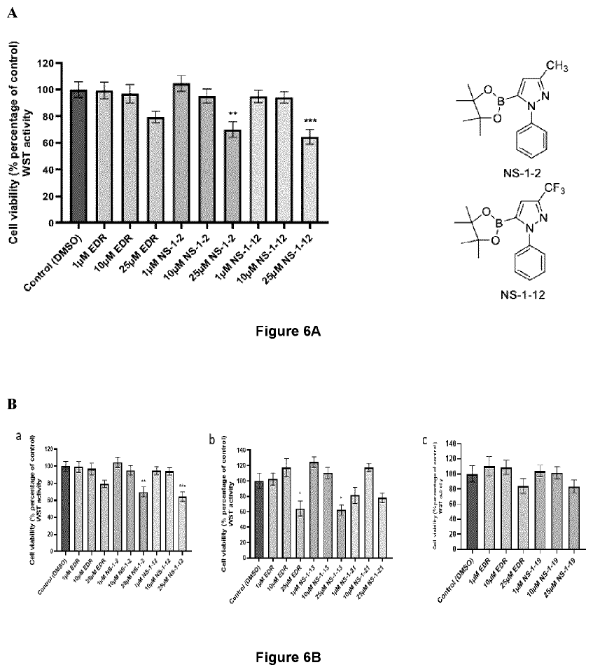

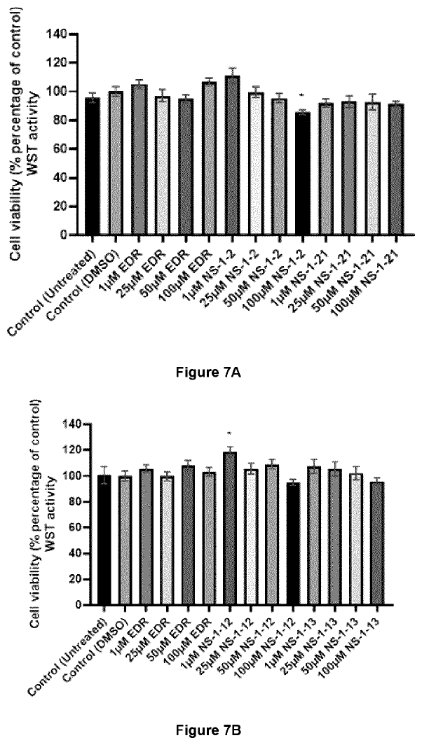

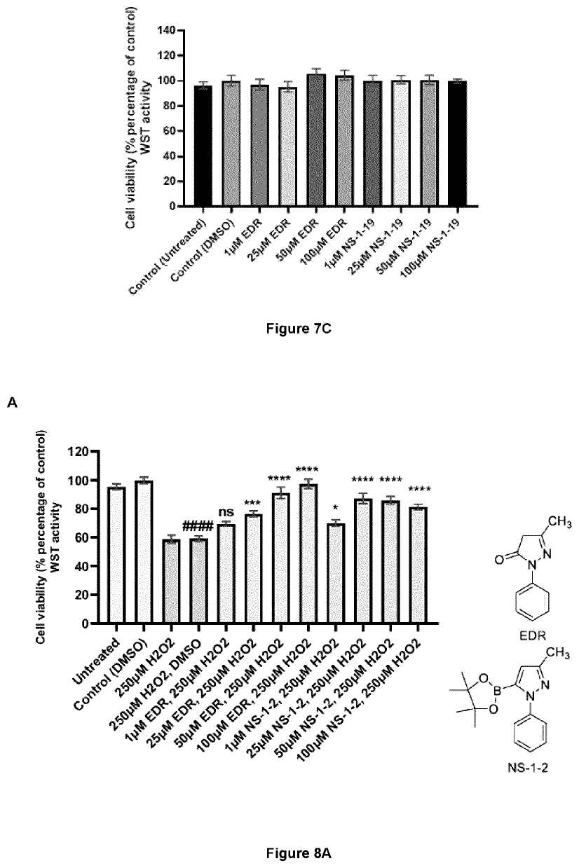

Methods: The in vitro properties and antioxidant abilities of the synthesized B⁵-EDR analogues were assessed in cell-based assays. The B⁵-EDR analogues were synthesized as described in Example 1, and neurotoxicity/neuroprotection of B⁵-EDR analogues, EDR and H₂O₂ was evaluated on (i) primary cortical neuronal cells (FIG. 6) (ii) neuroblastoma-spinal cord hybrid NSC-34 cells (FIGS. 7A-C and 8A-B); by WST-8 analysis (marker of neurotoxicity), in comparison to controls/EDR. The methodology employed for each of these assays is detailed below.

(i) Methodology for neurotoxicity/cell viability analysis in primary cortical neuronal cells (FIGS. 6A-B): Briefly, the cerebral cortex was isolated from fetus of mouse at about 17-about 18-day gestation. After meninges and blood vessels were removed, the tissue was digested with trypsin for about 15 min at about 37° C., then incubated with DNase for about another 15 min at about 37° C. and terminated with DMEM plus about 10% fetal bovine serum. After the supernatant was removed, the tissue was added with about 5-ml neurobasal medium (Life Technologies Inc) and triturated by gently pipetting. Neuronal cells contained in the supernatant were transferred to poly-D-lysine-coated plastic culture plates, adjusted to approximately 106 cell/ml, and cultured in neurobasal medium with GS21 supplements (Sigma-Aldrich Canada), 1× GlutaMax (Life Technologies Inc,) and about 1% penicillin-streptomycin at about 37° C. under about 5% CO₂. About 100 kcells/100 μl/well were seeded in 96 well plates for 2 Div. At 2 Div, the neurons were treated with different concentration of EDR (1, 10 and 25 μM) and EDR analogues NS-1-2, NS-1-12, NS-1-13, NS-1-19 and NS-1-21 (1, 10 and 25 μM) and incubated until 8 Div. On 8th Div of neurons, about 10 μl of WST reagent was added to each well and incubated for about 2 h. After about 2 h of incubation, the absorbance of soluble coloured formazan dye was measured calorimetrically at about 450 nM using microplate reader (Biotek instruments) with the background control as a blank. The cell viability was determined by comparing the absorbance of compounds treated cells to that of control cells. Data are representative of two independent experiments, with each measurement or dose tested six times.

(ii) Methodology for neurotoxicity/cell viability analysis in (NSC-34) cells (FIGS. 7A-C): Briefly, NSC-34 cells with about 20 k cells per well in 96 well plates were cultured in a complete medium consisting of high glucose Dulbecco's modified eagle medium (DMEM) (Thermo Fisher Scientific) supplemented with about 10% US-origin fetal bovine serum (Thermo Fisher Scientific), GlutaMAX-1, about 200 mM (100×) (Thermo Fisher Scientific), about 1% 100 mM Sodium pyruvate and about 1% 10,000 U/mL penicillin-streptomycin solution (Thermo Fisher Scientific) for about 20 h to reach confluency of about 70%—about 75%. All the cells were cultured in an incubator with about 5% of CO₂ at about 37° C. About 20 kcells/100 μl/well were seeded in 96 well plates for about 20 h in triplicates. Cells were treated with different concentration of compounds and incubated for about 20 h. About 10 μl of WST reagent was added to each well and incubated for about 2.5 h. After about 2.5 h of incubation, the absorbance of soluble coloured formazan dye was measured calorimetrically at about 450 nM using microplate reader (Biotek instruments) with the background control as a blank. The cell viability was determined by comparing the absorbance of compounds treated cells to that of control cells. All the experiments were repeated at least three times and measurements were run in triplicates.

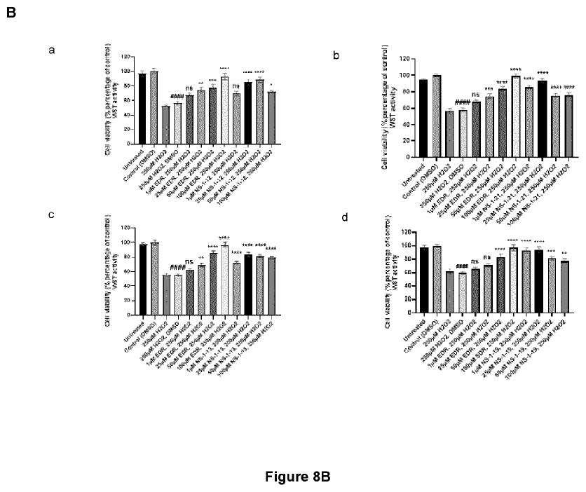

(iii) Methodology for neuroprotective effect/cell viability analysis in (NSC-34) cells (FIGS. 8A-B): Briefly, NSC-34 cells with about 20 k cells per well in 96 well plates were cultured in a complete medium consisting of high glucose Dulbecco's modified eagle medium (DMEM) (Thermo Fisher Scientific) supplemented with about 10% US-origin fetal bovine serum (Thermo Fisher Scientific), GlutaMAX-1, about 200 mM (100×) (Thermo Fisher Scientific), about 1% 100 mM Sodium pyruvate and about 1% 10,000 U/mL penicillin-streptomycin solution (Thermo Fisher Scientific) for about 20 h to reach confluency of about 70%—about 75%. All the cells were cultured in an incubator with about 5% of CO₂ at about 37° C. About 20 kcells/100 μl/well were seeded in 96 well plates for about 20 h in triplicates. After about 20 h, the media was changed, and the cells were pre-treated or prophylactically treated with different dose of EDR analogues and EDR for about 1 h. After about 1 h, the cells were treated with 250 μM of H₂O₂ for about 2 h. After a total period of about 3 h, about 10 μl of WST-8 reagent was added in each well and the absorbance readings were recorded after about 2.5 h at about 450 nm. All the experiments were repeated at least three times and measurements were run in triplicates.

### Results:

(i) Neurotoxicity/cell viability analysis in primary cortical neuronal cells: As seen in FIG. 6A, both of the tested analogues (NS-1-2 and NS-1-12) had similar cell viability results as EDR, and did not appear to decrease cell viability at concentrations of 1 μM and 10 μM. It was only at much higher concentrations of 25 μM that all compounds, including EDR, showed diminished cell viabilities. Thus, based on these results, the B⁵-EDR analogues do not appear to reduce cell viabilities to a greater degree than EDR. As shown in FIG. 6B, lower concentrations (10 μM and 1 μM) of EDR analogues NS-1-2, NS-1-12, NS-1-21, NS-1-19, NS-1-13 and EDR showed good cell viability of around 100% on PCNC compared to controls. In addition, the cell viability of analogues are like that of EDR. Moreover, higher concentrations (25 μM) of EDR and EDR analogues showed around (70±10%) decrease in cell viability on PCNC compared to control (DMSO).

(ii) Neurotoxicity/cell viability analysis in (NSC-34) cells: As shown in FIG. 7A, prophylactic treatment of both EDR and NS-1-2 protected NSC-34 cells from loss of cell viability induced by 250 μM H₂O₂, and are neuroprotective against H₂O₂ induced oxidative stress. EDR and NS-1-2 were found to both be neuroprotective in this assay in PCNC and NSC-34 cells (NSC-34 shown in FIG. 7A). As shown in FIG. 7B, the synthesized EDR analogues showed increased viability with no cytotoxicity and neurotoxicity compared to control groups. In addition, all of the test compounds showed viability of around 90-100% in almost every concentration (1 to 100 μM). Also, much like the PCNC study above, the test compounds showed equal viability compared to EDR group. Finally, application of EDR and EDR analogues from 1 to 100 μM showed no neurotoxicity and cytotoxicity towards NSC-34 motor neuronal cells. Based on these viability results, EDR analogues were evaluated for neuroprotectivity in the experiments described in (iii). The results of these experiments are provided below.

(iii) Neuroprotective effect/cell viability analysis in (NSC-34) cells: As shown in FIGS. 8A-B, EDR showed significant H₂O₂ scavenging effects in a dose dependent manner. In addition, the results showed that all five EDR analogues (NS-1-2); (NS-1-13); (NS-1-21); (NS-1-19); (NS-1-12) showed almost equal viability at 50 μM dose compared to EDR (this was statistically significant). Moreover, all five EDR analogues tested (NS-1-2); (NS-1-13); (NS-1-21); (NS-1-19); (NS-1-12) showed better neuroprotection in scavenging neurotoxic effects of H₂O₂ compared to EDR at lower doses of 25 μM. This suggests that EDR analogues are more potent than EDR in scavenging H₂O₂ at lower doses.

In addition, four EDR analogues (NS-1-2); (NS-1-13); (NS-1-21); (NS-1-19) showed better neuroprotection in scavenging neurotoxic effects of H₂O₂ compared to EDR at lower doses of 1 μM; suggesting, again, that EDR analogues are more potent than EDR in scavenging neurotoxic effects of H₂O₂ at lower doses. Finally, with higher neuroprotection at lower doses of 1 μM and 25 μM and equal neuroprotection at 50 μM, higher doses of these analogues i.e., 100 μM, showed (10-15%) less viability compared to EDR.

### Example 3 Evaluation of Therapeutic Index and Pharmacokinetic Properties of Boron-Based EDR Analogues

The synthesized B⁵-EDR analogues may also serve as dual-role therapeutic agents, via the benefits of the boron functionality and by serving as a prodrug for EDR leading to a similar or improved therapeutic index to EDR and improved pharmacokinetic properties.

Methods: To evaluate the pharmacokinetics and biodistributions of the B5-EDR analogues, in comparison to EDR, the initial studies focus on single IV dose acute administration in rats using the most promising lead compounds. Using a population PK approach, rats (n=2 per time point) are dosed by tail-vein IV with the novel compound and sacrificed at 10, 20, 30, 60, 120, mins then 4 hr, 8 hr, 12 hr and 24 hr. Blood is drawn at time of sacrifice and the drug's concentration-time curve is constructed for the cohort by cleaning up blood with liquid-liquid extraction and performing LC/MS for compound levels. Certain parameters of AUC, Cmax, Kel and Tpeak are determined using pharmacokinetic modelling. Organs including brain, heart, kidney, liver, pancreas, spleen, and stomach are collected and samples of organs homogenized (1:3 in Tris-HCl containing protease inhibitor cocktail), liquid:liquid extraction performed and then analysed by LC/MS for levels of compound in the organs. This will ensure there is no preferential compartmentalization of drug. Gross necropsies and fine microscopy is also performed, overall, yielding basic safety pharmacology/toxicology data. An additional cohort of rats are dosed by oral gavage and further analysis are performed to determine oral pharmacokinetic and biodistribution data. A method is also developed to detect EDR in vivo (rats & SOD1-G37R) as a biomarker proof-of-concept that the B5-EDR compounds serve as prodrugs of EDR, including kinetics. Following rat PK studies, similar experiments are performed using a single IP dose acute administration in SOD1-G37R mice. Also, the activity of UGT and CYP enzymes towards the novel compounds are evaluated using a reaction phenotyping approach with substrate depletion. Using certain mass spectrometry conditions, the metabolic profile of selected B5-EDR analogues will be surveyed by UGT and CYP enzymes to identify likely metabolic pathways. Briefly, novel B5-EDR analogues are placed into test tubes with buffer, recombinant active hepatic UGT (UGT1A1, 1A3, 1A4, 1A6, 1A9, 2B4, 2B7, 2B15, 2B17) and CYP (1A2, 2C9, 2C19, 2D6, 3A4) isoforms and the co-activator UDPGA or NADPH as necessary. The supernatant is analyzed by mass spectrometry and loss of compound is compared to an initial spiked tube that contained no co-factor but was incubated under the same conditions. Positive controls are used for each selective/specific substrate e.g., for UGT1A1, bilirubin is the positive control. Where no loss of compound is observed, the compound is declared “not a substrate”. Where metabolism is observed, kinetic curves are determined (e.g. either Michaelis-Menten or Sigmoidal kinetics). The Km and Vmax parameters are determined, and this will enable estimation of intrinsic clearance. Intrinsic clearances can generally be used for CYP isoforms to gain a good measure of in vivo clearance. Additional experiments are performed to examine the p-glycoprotein liabilities[42][43] of the B5-EDR analogues including studies to assess penetration of the blood brain barrier[44][45].

### Example 4 In Vivo Studies of B5-EDR Analogues in Mouse Model of ALS

The synthesized B⁵-EDR analogues may serve as candidate drugs for the development of effective therapeutic(s) for ALS patients. Therefore, therapeutic efficacy and acute toxicity was evaluated in a mouse model of ALS.

Methods: B⁵-EDR analogues were synthesized as described in Example 1. The specific methodology employed for this Example is described below. All the statistical analyses were carried out by using GraphPad Prism 8 software (version 9.5.1 (733)), (GraphPad Software, La Jolla, CA). Statistical significance was performed using a two-tailed unpaired t-test used for body weight assessment, disease onset, survival, and weight loss comparisons. Kaplan-Meier survival analysis and log rank test was also used for survival analysis. For these in vivo studies, disease onset or symptom onset was defined by two criteria. The first criteria is defined by the loss of 10% body weight based on the highest recorded weight during the age of the mice after pre symptom onset treatment study. The loss of 10% body weight is often accompanied by appearance of symptoms of muscle weakness and the second criteria of disease onset, which is retrospectively defined as the age at which the mouse reaches peak weight. Mice were assessed and monitored daily by trained staff for weight and muscle weakness starting at 90 days of age i.e., well before clinical onset of disease. According to the common consensus among ALS scientific community, the disease onset is defined as a time when mice have reached their peak body weight well before the denervation induced muscle atrophy and weight loss.

### (i) Mice and Tissue Preparation:

Transgenic mice carrying human G37R mutant SOD1 [B6.Cg Tg(SOD1*G37R)42Dpr/J] were obtained from the Jackson Laboratory (Bar Harbor, ME, USA). These mice were crossed with female mice with a C57BL/6 background for at least four generations. Colonies are maintained in the Central Animal Care Services (CACS), University of Manitoba. The mice were used in accordance with the Guide of Care and Use of Experimental Animals of the Canadian Council on Animal Care. Transgenic offspring were genotyped by PCR of DNA obtained from ear biopsies, see (ia) below, using a protocol provided by the Jackson Laboratory.

### (ia) DNA Isolation and Genotyping

Ear samples were lysed overnight at about 55° C. in about 300 μl of TNES buffer (about 1 M Tris, about pH 8.5, about 0.5 M EDTA, about 10% SDS, about 5 M NaCl, distill water) and about 20 μg/μl of Proteinase K (Sigma). An about equal volume of phenol/chloroform (about 1:1) was added to the mixture, and mixed gently. Then debris was separated from the samples by centrifugation for about 15 minutes at about 14,500 rpm, and the supernatant containing DNA was collected. DNA was precipitated using an about equal volume of cold about 95% ethanol 47 (−20° C.), and the DNA was pelleted via centrifugation for about 10 minutes at about 14,500 rpm. The supernatant was discarded, and the pellet was washed with about 70% ethanol. The centrifugation was repeated to discard the ethanol. Tubes were placed in the ventilating hood to evaporate residual ethanol for about 1 hour. Then the DNA was resuspended in about 30 μl distilled water and stored at about 4° C. until the genotype was determined using PCR. Isolated DNA was used to determine the genotypes of progeny. Approximately 450 mice were genotyped from the breeding. About 19 μl of ultra-pure water (ThermoFisher), about 2.5 μl of 10×PCR buffer (about 200 mM Tris-HCl about pH 8.4, about 500 mM KCl) (ThermoFisher), about 1 μl of about 50 mM MgCl2, about 0.25 μl of about 10 mM dNTPs, about 0.5 μl of about 10 μM forward primer (5′-CATCAGCCCTAATCCATCTGA-3′), about 0.5 μl of about 10 μM reverse primer (5′-CGCGACTAACAATCAAAGTGA-3′), and about 0.25 μl of about 5 U/μl Taq DNA polymerase (ThermoFisher) was added to about 2 μl of sample DNA. PCR conditions for the above reactions included a three-minute initial denaturation at about 95° C., followed by about 35 cycles of an about 30-second denaturation step at about 95° C., an about 30-second annealing step at about 55° C., and an about 45-second extension step at about 73° C. And then an about 10-minute final extension step at about 72° C. PCR products mixed with gel red were separated on an about 1% agarose gel. The gel was then visualized in an G:BOX imager using GeneSys imager software (Syngene, UK).

### (ii) Group Assignment, Drug Formulation, Storage, and Administration of Compound Group Assignment for Determination of Acute Toxicity in WG37R Mice:

The WG37R mice were randomly assigned to 2 groups: For the first set of the experiment, a total of 6 animals per group (3 male and 3 female) were used in the study. Group 1: Sham treatment 1 (1:20 DMSO/PBS) and Group 2: NS-1-2 (10 mg/kg body weight). According to requirement of acute toxicity study, 6-10 animals should be used to evaluate the effect of a substance. However, as the new analogues are structurally similar to Edaravone, having a single-dose safety profile of 450 mg/kg, the mortality of the Edaravone analogues at a dose of 10 mg/kg body weight/day was not expected. Considering the above facts, for the present acute study, the minimum number of animals was 6 per group. Morphological alterations, histological changes, and mean body weight assessments for whole set of animals, for both the WG37R control groups (N=6; 1:20, DMSO:PBS) and WG37R treated groups (N=6; NS-1-2; 10 mg/kg bodyweight) were assessed during the 14-day acute toxicity assessment, by giving a single IP injection to the mouse at 2 months of age (see further detail in (iia) below.

### (ii) Group Assignment, Drug Formulation, Storage, and Administration of Compound Group Assignment for Determination of Chronic Toxicity in WG37R Mice:

The WG37R mice were randomly assigned to 2 groups: For the second set of the experiment, a total of 6 animals per group (3 male and 3 female) were used in the study. Group 3: Sham treatment 1 (1:20 DMSO/PBS) and Group 4: NS-1-2 (10 mg/kg body weight). According to requirement of acute toxicity study, 6-10 animals should be used to evaluate the effect of a substance. However, as the new analogues are structurally similar to Edaravone, having a single-dose safety profile of 450 mg/kg, the mortality of the Edaravone analogues at a dose of 10 mg/kg body weight/day was not expected. Considering the above facts, for the present acute study, the proposed minimum number of animals was 6 per group. Morphological alterations, histological changes, and mean body weight assessments for whole set of animals, for both the WG37R control groups (N=5; 1:20, DMSO:PBS) and WG37R treated groups (N=6; NS-1-2; 10 mg/kg bodyweight) were assessed during the 120-day chronic toxicity assessment, by giving daily of 120 IP injection to the mouse at 3 months of age. Only one male mice from group 3 (control) was terminated unexpectedly, and was likely due to an accidental error in injection (discussed with Central Animal Care Services, veterinarians and staff). IP injections may be difficult because of the aggressiveness of mice (particularly the male mice, during physical restraint). In total, 1440 (12×120) daily IP injections were proposed for the chronic toxicity assessment (see further detail in (iia) below).

### (Iia) Drug Formulation, Storage, and Route of Administration Determination of Acute and Chronic Toxicity:

For intraperitoneal administration, about 1:20 of DMSO/PBS, at a pH of about 7.4 was used for the suspension of NS-1-2 test substance. Edaravone was dissolved in about 1 mL of about 1 mol/L NaOH and adjusted to about pH 7.4 by adding about 1 mol/L HCl, and then diluted in saline. A volume of about 400 μl from final reconstituted solution was aliquoted and stored in about −80° C., until further use. For acute toxicity experiments, a single dose of about 10 mg/kg bodyweight of NS-1-2 was intraperitoneally injected to group 2, WG37R mice model at the age of 2 months. The WG37R mice in the group 1 received an injection of equal volume of (about 1:20; DMSO:PBS) instead. For chronic toxicity experiments, 120 daily doses of about 10 mg/kg bodyweight of NS-1-2 was intraperitoneally injected to group 4, WG37R mice model at the age of 3 months for 120 days, until the age of 7 months. The WG37R mice in the group 3 received an injection of equal volume of (about 1:20; DMSO:PBS) instead.

(iib) Hematoxylin and Eosin (H&E) Staining and Microscopy

Wild type (WT) G37R mice received a single dose (about 0.2 mls) IP injection of Sham suspended in (about 1:20; DMSO:PBS) and a single dose of about 10 mg/kg bodyweight (about 0.2 mls) IP injection of NS-1-2 suspended in about 1:20; DMSO:PBS at the age of 2 months. The mice were observed for the period of 2 weeks. At the experimental endpoint, the mice were first deeply anesthetized with a mixture of about 20% v/v isoflurane/propylene glycol (about 1 ml of the mixture per about 500 ml of bell jar space, University of Manitoba animal care SOP A003) and than exsanguination was done by cutting the animal's right atrium followed by an intracardiac perfusion with about 0.9% NaCl. During perfusion, syringe barrel nosecone was used for prolonged anesthesia. Cardiac perfusion was followed by perfusion with about 4% paraformaldehyde for histology (H&E staining) analysis. Mouse tissues (brain, heart, spinal cord, kidney, liver, muscle, lung, and spleen) were fixed for about 48 h in about 4% buffered formalin at about 4° C. Only the spinal cord was processed after about 24 h to remove the spine and again fixed for about another 24 h. Samples were processed and embedded into paraffin blocks at the Histomorphology and Ultrastructural Imaging platform, Dept. of Human Anatomy and Cell Science, University of Manitoba. Briefly, the embedded tissues were cut into about 5 μm thickness, mounted on super frost plus slides and dried overnight at about 37° C. Slides were deparaffinized in about 2 changes of xylene and rehydrated in descending alcohols (about 2 changes of about 100% Ethanol and about 2 changes of about 95% Ethanol) and tap water. Slides were stained with Harris Haematoxylin followed by differentiation with acid alcohol. After rinsing in tap water, saturated lithium carbonate was used for Bluing the nucleus. After that the slides were rinsed in tap water and counter stained with eosin. Following eosin staining, the slides were dehydrated using ascending alcohols, cleared with xylenes and mounted coverslips on sections with paramount.

The mounted slides were then visualized, and the images were taken using Axioskop 2 mot plus microscope using AxioVision software version 4.8 (Carl Zeiss, Inc., Thornwood, NY).

Similar to the acute toxicity experiments, hematoxylin and eosin (H&E) staining and Microscopy experiments were done for the animals assigned in chronic toxicity groups (data not presented here).

(iii) Group Assignment for Determination Therapeutic Efficacy of B5-EDR Analogues, in Direct Comparison to Control/Sham, in Decreasing Weight Loss (Cachexia), Delaying Disease Onset, and Increasing Survival in the SOD1-G37R Mouse Model of ALS.

For these experiments, a set of experiments was carried out having two groups of mice:
Group 1: Sham treatment (1:20 DMSO/PBS); and
Group 2: NS-1-2 treatment (1:20 DMSO/PBS) (10 mg/kg body weight/day).
   For these experiments, age matched Het G37R (Line 42) mice were administered vehicle (1:20, DMSO:PBS) or treatment (NS-1-2; 10 mg/kg bodyweight), daily, starting at the age of 90 days until the age of 210 days, and monitored daily for several significant humane point indicators including, a) Mouse unable to right itself in 15 sec b) 25% loss of weight on the highest recorded weight c) Full paralysis of one or more hind limbs d) Loss of bladder functions e) Crusty eyes/loss of vision and/or f) Penile prolapse.

For weight loss experiments, weight loss was based on highest recorded weight (humane end point).

For disease onset experiments, symptom onset was assessed (i.e. loss of 10% body weight based on the highest recorded weight with muscle weakness and time to peak body weight (days), was assessed and monitored daily by trained staff starting at 90 days of age i.e., well before pre-symptom onset of disease also, monitored daily for several significant humane point indicators including a) Mouse unable to right itself in 15 sec b) 25% loss of weight on the highest recorded weight c) Full paralysis of one or more hind limbs d) Loss of bladder functions e) Crusty eyes/loss of vision f) Penile prolapse. One tailed (unpaired t test) was performed to analyse age to reach (10%) loss of body weight.

Also, injection sites were alternated between the right and left sides of animal to reduce pain/inflammation. To see an effect of treatment in these experiments, a sample size of 12 per group is used. The mean deviation of oxidative SOD1 level is 20%. At least 30% reduction of oxidative SOD1 with the treatments, with S/N ratio of 1.5, a power of 0.9 and a significance level of 0.05, the required sample size is 12. If any less are used, the data would not be statistically significant. This study will include two further groups: Sham treatment (1M NaOH) and Edaravone in 1M NaOH (10 mg/kg body weight/day). When these groups are added to the experimental protocol, a total of 12 animals per group (6 male and 6 female) will be used. Thus, a total of 48 mice will be used when all of the experiments are conducted.

### Results:

### Determination of Acute Toxicity in WG37R Mice:

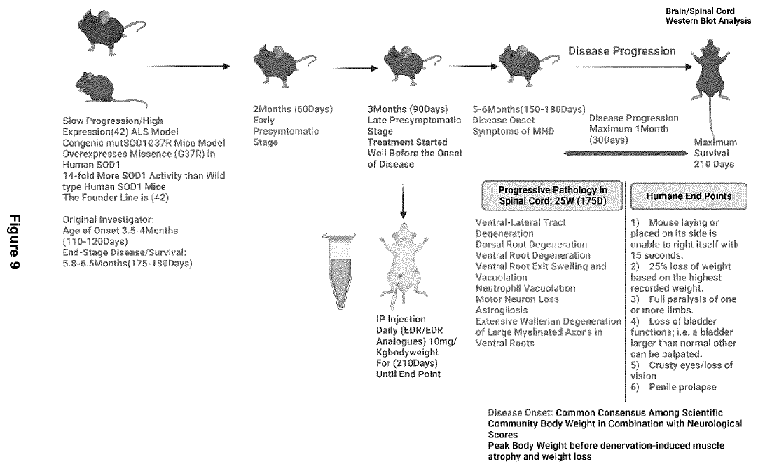

(i) Morphological Alteration: After receiving a single dose of 10 mg/kg bodyweight (about 0.2 mls) IP injection of NS-1-2 suspended in (about 1:20; DMSO:PBS) at the age of 2 months, the mice were observed for a period of 2 weeks. Daily monitoring for various significant indicators for humane endpoints like weight, appearance, body condition, general behaviour (see FIG. 9 for examples of humane end points and method (iii) above) was started from the first day of treatment until 14th day. No acute treatment-related deaths occurred during the 2 weeks monitoring period. Daily general observations showed normal appearance, normal general behaviour with well-conditioned body condition. Furthermore, a single dose of 10 mg/kg bodyweight of NS-1-2 did not change the skin, fur colors, eyes, mucous membrane, the occurrence of secretions and excretions, motor activities, or autonomic activity of the mice tested (data not shown).

Chronic toxicity studies are performed with daily dosing for 120 days starting at the age of 3 months until the age of 7 months. These experimental results are similar to the acute toxicity studies described above and have 6 animals per group. IP administration of NS-1-2 (10 mg/kg/day) daily for 120 days, with (total of 120 IP IJ, and 1200 mg of total dose over 120 days), was seemingly tolerated by wildtype G37R mice. No chronic treatment-related deaths occurred during 4 months of the monitoring period. Daily general observations showed normal appearance, normal general behaviour with well-conditioned body condition. Further, a total of 120 dose of 10 mg/kg bodyweight of NS-1-2 did not change the skin, fur colors, eyes, mucous membrane, the occurrence of secretions and excretions, motor activities, or autonomic activity of the mice tested (data not shown). Daily, chronic IP administration of NS-1-2 (10 mg/kg/day) daily for 120 days, was seemingly tolerated by wildtype G37R mice. Furthermore, NS-1-2-treated mice developed no clinical signs of toxicity including a decrease in mean weight, hunched posture, orbital tightening, piloerection, and low activity compared to vehicle-treated (1:20, DMSO:PBS) mice.

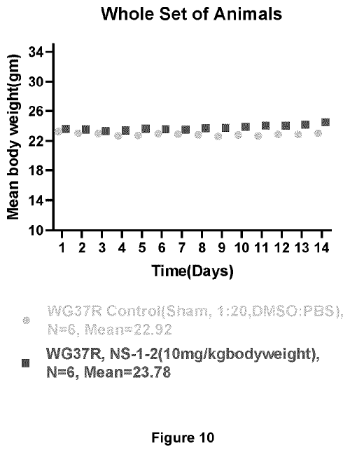

(ii) Assessment of Body Weight (Acute toxicity assessment): As shown in FIG. 10, the acute treatment of EDR analogue NS-1-2 at a dose of 10 mg/kg bodyweight in wild type G37R model did not induce any abnormal changes in the body weight of the mice. Moreover, there was no significant difference in the changes of body weight between the control group and the treatment groups, which further supports the absence of toxicity.
(ii) Assessment of Body Weight (Chronic toxicity assessment): As shown in FIG. 11, the chronic treatment of EDR analogue NS-1-2 at a dose of 10 mg/kg bodyweight for 120 days in wild type G37R model did not induce any abnormal changes in the body weight of the mice. Moreover, there was no significant difference in the changes of body weight between the control group and the treatment groups, which further supports the absence of toxicity.
(iii) Histology analysis: As shown in FIG. 12, G37RWT male mice, that received a single intraperitoneal (IP) injection of NS-1-2 at a dose of 10 mg/kg bodyweight, beginning at 60 days of age, showed no significant differences with respect to control mice, in the stained tissue samples. In addition, there were no signs of overt degeneration, inflammation and necrosis in any of examined tissues. In particular, for the liver, control male and treated (NS-1-2) male mice both illustrated normal lobular architecture with central veins and radiating hepatic cords. Hepatocytes were observed to be normal and there were no signs of inflammatory response in both the control and NS-1-2 treated liver tissues. For the kidney, control male and treated (NS-1-2) male mice both illustrated normal glomerular structure of the kidney and no pathological changes were observed between either group. For the spleen, control male and treated (NS-1-2) male mice both illustrated normal micro-architecture of the white and red pulp with no morphological alteration. For the heart, control male and treated (NS-1-2) male mice both had normal myocardium morphology. For the lungs, control male and treated (NS-1-2) male mice both displayed normal lung architecture with no signs of alteration in alveolar architecture. For the muscle, control male and treated (NS-1-2) male mice both illustrated normal homogeneous distribution of polygonal shaped muscle fiber with peripheral nuclei. There was no degeneration of fibres and both groups had normal morphology. For the spinal cord, control male and treated (NS-1-2) male mice both illustrated normal morphology with central canal, neurons and glial cells. For the brain, control male and treated (NS-1-2) male mice both illustrated normal morphology of the hippocampus and demonstrated the regular architecture of the CA3 region where the pyramidal cell layer neurons (P) were found to be uniform in size and evenly arranged.

Similarly, as shown in FIG. 13, G37RWT female mice that received a single intraperitoneal (IP) injection of NS-1-2 at a dose of 10 mg/kg bodyweight, beginning at 60 days of age, showed no significant differences with respect to control mice, in the stained tissue samples. In addition, there were no signs of overt degeneration, inflammation and necrosis in any of examined tissues. In particular, for the liver, control female and treated (NS-1-2) female mice both illustrated normal lobular architecture with central veins and radiating hepatic cords. Hepatocytes were observed to be normal and there were no signs of inflammatory response in both the control and NS-1-2 treated liver tissues. For the kidney, control female and treated (NS-1-2) female mice both illustrated normal glomerular structure of the kidney and no pathological changes were observed in either group. For the spleen, control female and treated (NS-1-2) female mice both illustrated normal micro-architecture of the white and red pulp with no morphological alteration. For the heart, control female and treated (NS-1-2) female mice both had normal myocardium morphology. For the lungs, control female and treated (NS-1-2) female mice both displayed normal lung architecture with no signs of alteration in alveolar architecture. For the muscle, control female and treated (NS-1-2) female mice both illustrated normal homogeneous distribution of polygonal shaped muscle fiber with peripheral nuclei. There was no degeneration of fibres and both groups had normal morphology. For the spinal cord, control female and treated (NS-1-2) female mice both illustrated normal morphology with central canal, neurons and glial cells. For the brain, control female and treated (NS-1-2) female mice both illustrated normal morphology of the hippocampus and demonstrated the regular architecture of the CA3 region where the pyramidal cell layer neurons (P) were found to be uniform in size and evenly arranged.

### Determination Therapeutic Efficacy of B5-EDR Analogues:

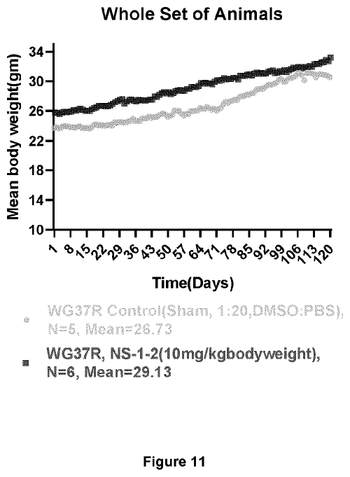

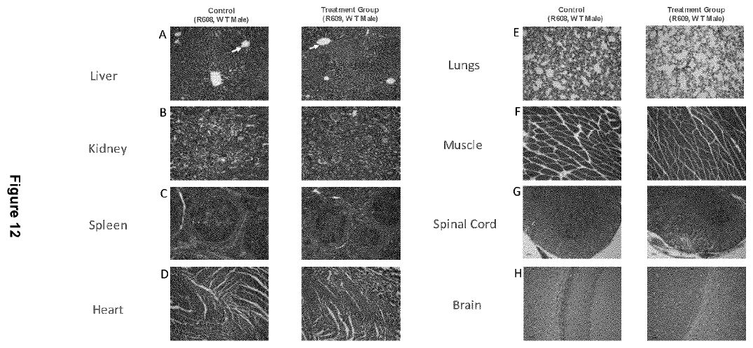

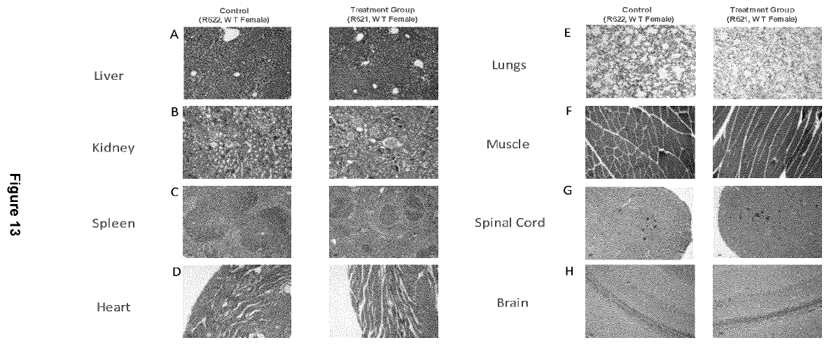

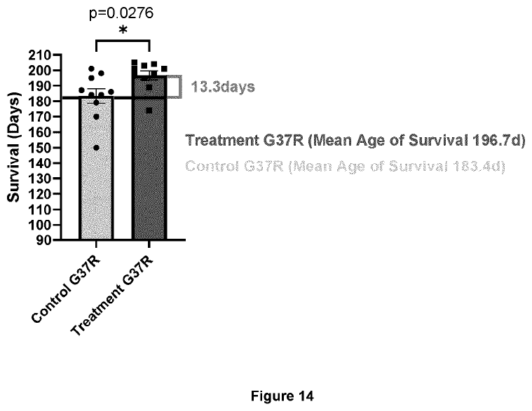

(i) Survival: Survival of the mice receiving the EDR analogue(s) was also assessed. It was found that daily IP injections starting from the age of 90 days until the age of 210 days in Het G37R (Line 42) mice prolonged the survival in treated Het G37R (Line 42) mice (n=10/treatment) as compared to non-treated mice (N=10/control). As shown in FIG. 14, the mean age of survival was prolonged from 183.4 days for non-treated mice (n=10/control) to 196.7 days for treated mice (n=10/treatment). This value was statistically significant (P=0.0276) using a two-tailed unpaired t test.

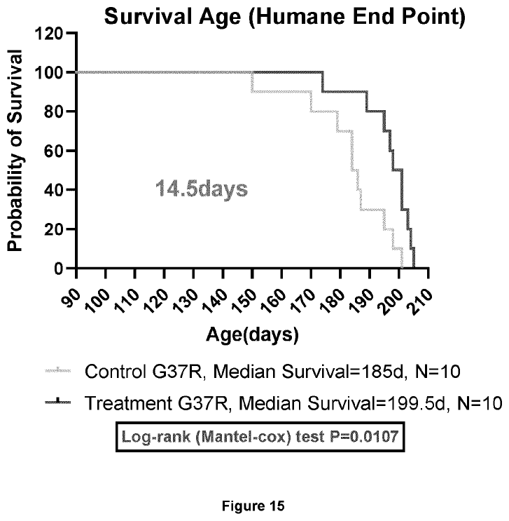

Given the difference in survival of 13.3 days or almost two weeks, it appears that the B⁵-EDR compound NS-1-2 may serve as an effective therapeutic in the G37R mouse model. In addition, as shown in FIG. 15, using a Kaplan-Meier Log-rank (Mantel-cox) test for percent survival (Humane end point) in Het G37R (Line 42) ALS model mice, it was found that daily intraperitoneal injection (IP,IJ) starting from the age of 90 days until the age of 210 days in mice prolongs the median survival from 185 days for non-treated mice (n=10/control) to 199.5 days for treated mice (n=10/treatment). This was statistically significant (Log-rank (Mantel-cox) test: P=0.0107). Further, Kaplan-Meier survival curves show that long-term treatment with NS-1-2 prolongs the survival age or life span of mutant G37R ALS mice compared to control mutant G37R ALS mice by 14.5 days. The survival data has also been represented in tabulated form (Table 1) below.

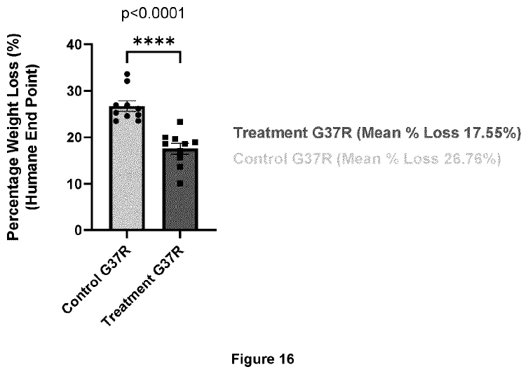

(ii) Weight loss: Weight loss changes, as representative of ALS progression, was also assessed in the mice. As shown in FIG. 16, daily intraperitoneal injection (IP,IJ) of NS-1-2 treatment starting from the age of 90 days until the age of 210 days significantly prevents percentage mean weight loss (humane end point) or percentage ALS induced cachectic weight loss (humane end point) from 26.76% for non-treated mice (n=10/control) to 17.55% for treated mice (n=10/treatment). Thus, these results demonstrate that six out of ten animals in the control group had lost >25% bodyweight at humane endpoint and the remaining four out of ten animals had lost >23.5% bodyweight), while in the treatment group, none of the animals lost >25% bodyweight, nor did any treatment animals lose >23.5% bodyweight, indicating that the treatment group resisted bodyweight changes; a classic hallmark of ALS progression. Further, the weight loss is regarded as the predictor of shorter survival. The weight loss data has also been represented in tabulated form (Table 1) below.

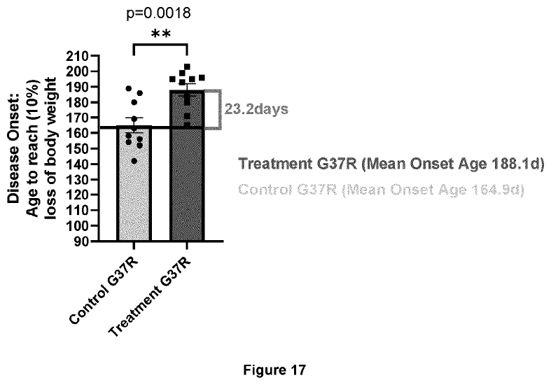

(iii) Disease onset (Age to 10% weight loss (with muscle weakness)): Disease onset was also assessed in the mice by measuring the age to reach 10% loss of body weight based on highest recorded weight (with muscle weakness). As shown in FIG. 17, daily intraperitoneal injection (IP, IJ) starting from the age of 90 days until the age of 210 days in mice delayed the onset of disease, the mean age (loss of 10% body weight) 164.9 days for mice (n=10/control) to 188.1 days for mice (n=10/treatment). Further, the disease onset was delayed by around 23.2 days in NS-1-2 treatment group compared to control group with no treatment. This was statistically significant (P=0.0018) using a two-tailed unpaired t test. Thus, the EDR analogue NS-1-2 can delay onset of disease in direct comparison to control group. The disease onset data has also been represented in tabulated form (Table 1) below.

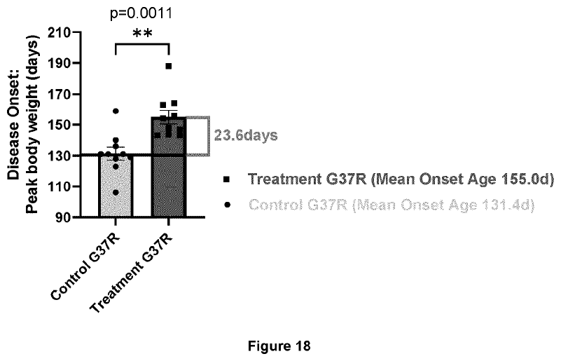

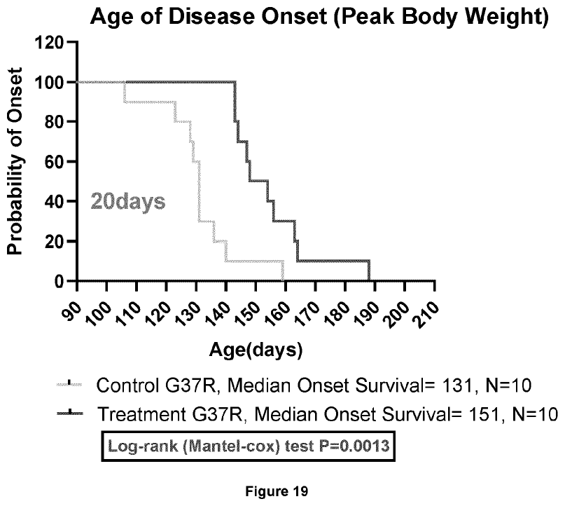

(iv) Disease onset (Age to reach peak body weight (days)): Disease onset is retrospectively defined as the age at which the mouse reaches peak weight. As shown in FIG. 18, daily intraperitoneal injection (IP, IJ) starting from the age of 90 days until the age of 210 days in mice delayed the onset of disease, the mean age (time to peak body weight) 131.4 days for mice (n=10/control) to 155 days for mice (n=10/treatment). Further, the disease onset was delayed by around 23.6 days in NS-1-2 treatment group compared to the control group with no treatment. This was statistically significant (P=0.0011). Thus, the EDR analogue NS-1-2 can delay onset of disease in direct comparison to the control group. In addition, as shown in FIG. 19, using a Kaplan-Meier Log-rank (Mantel-cox) test for probability of onset (age to reach peak body weight) in Het G37R (Line 42) ALS model mice, it was found that daily intraperitoneal injection (IP, IJ) starting from the age of 90 days until the age of 210 days in mice delayed the median survival from 131 days for non-treated mice (n=10/control) to 151 days for treated mice (n=10/treatment). This was statistically significant (Log-rank (Mantel-cox) test: P=0.0013). Further, Kaplan-Meier survival curves show that treatment with NS-1-2 delayed the disease onset of mutant G37R ALS mice compared to control mutant G37R ALS mice by 20 days.

The disease onset data has also been represented in tabulated form (Table 1) below.

Table 1 below represents a compilation of the data provided in the therapeutic efficacy studies described above for results (i)-(iv).

TABLE 1
Comparison of therapeutic efficacy of B⁵-EDR analogue (NS-1-2) to control/sham
To Study the Therapeutic Effects of Edaravone Analogues (NS-1-2) in the SOD1-G37R Mouse Model of Amyotropic Lateral Sclerosis
Protocol# 21-014, AC11693
Whole Set of Animals (Male/Female)
Sham Treatment (N = 10)

| | | | Disease Onset: Age to reach | Age to reach 10% loss of body | 25% Loss of Weight Based |
|---|---|---|---|---|---|
| | | Control | peak body weight (days) | weight based on highest recorded | on Highest Recorded Weight |
| | | G37R | (Mice was assessed and | weight with muscle weakness) | (Humane End Point) |
| | | (Survival | monitored daily by | (Mice was assessed and monitored | (Mice was assessed and monitored |
| | | Days) | trained staff for weight | daily by trained staff for weight | daily by trained staff for weight and |
| Animal | | (Humane | and other significant | and other significant humane | other significant humane point indicators |
| ID | Sex | End Point) | humane point indicators) | point indicators) | Weight Loss (%): |
| R651RR | Male | 195 | 131 | 180 | Weight Loss >25% |
| | | | | | Weight Loss (%): 25.324 |
| R644LL | Male | 184 | 129 | 169 | Weight Loss <25% |
| | | | | | Weight Loss (%): 23.52 |
| R624R | Male | 179 | 140 | 156 | Weight Loss >25% |
| | | | | | Weight Loss (%): 33.6 |
| R712LL | Male | 170 | 131 | 158 | Weight Loss <25% |
| | | | | | Weight Loss (%): 24.60 |
| R751L | Male | 184 | 128 | 152 | Weight Loss >25% |
| | | | | | Weight Loss (%): 26.17 |
| R647LL_R | Female | 186 | 136 | 149 | Weight Loss >25% |
| | | | | | Weight Loss (%): 32.107 |
| R697LL | Female | 187 | 106 | 163 | Weight Loss >25% |
| | | | | | Weight Loss (%): 26.923 |
| R699R | Female | 198 | 131 | 186 | Weight Loss <25% |
| | | | | | Weight Loss (%): 23.58 |
| R705LR | Female | 150 | 123 | 142 | Weight Loss >25% |
| | | | | | Weight Loss (%): 26.90 |
| R703LL | Female | 201 | 159 | 189 | Weight Loss <25% |
| | | | | | Weight Loss (%): 24.88 |

NS-1-2 Treatment (N = 10)

| | | | Disease Onset: Age to reach | Age to reach 10% loss of body | 25% Loss of Weight Based |
|---|---|---|---|---|---|
| | | Treatment | peak body weight (days) | weight based on highest recorded | on Highest Recorded Weight |
| | | G37R | (Mice was assessed and | weight with muscle weakness) | (Humane End Point) |
| | | (Survival | monitored daily by | (Mice was assessed and monitored | (Mice was assessed and monitored |
| | | Days) | trained staff for weight | daily by trained staff for weight | daily by trained staff for weight and |
| Animal | | (Humane | and other significant | and other significant humane | other significant humane point indicators |
| ID | Sex | End Point) | humane point indicators) | point indicators) | Weight Loss (%): |
| R627R | Male | 201 | 143 | 199 | Weight Loss <25% |
| | | | | | Weight Loss (%): 17.056 |
| R711R | Male | 201 | 143 | 171 | Weight Loss <25% |
| | | | | | Weight Loss (%): 18.91 |
| R715LR | Male | 205 | 156 | 184 | Weight Loss <25% |
| | | | | | Weight Loss (%): 19.67 |
| R736L | Male | 189 | 147 | 180 | Weight Loss <25% |
| | | | | | Weight Loss (%): 20 |
| R739RR | Male | 195 | 154 | 193 | Weight Loss <25% |
| | | | | | Weight Loss (%): 15.68 |
| R638L | Female | 198 | 164 | 195 | Weight Loss <25% |
| | | | | | Weight Loss (%): 18.543 |
| R639R | Female | 203 | 163 | 203 | Weight Loss <25% |
| | | | | | Weight Loss (%): 10.122 |
| R605L | Female | 197 | 188 | 196 | Weight Loss <25% |
| | | | | | Weight Loss (%): 13.66 |
| R700LR | Female | 204 | 148 | 195 | Weight Loss <25% |
| | | | | | Weight Loss (%): 18.548 |
| R701L | Female | 174 | 144 | 165 | Weight Loss <25% |
| | | | | | Weight Loss (%): 23.34 |

The Examples provided herein suggest that the boron-based EDR analogues can have neuroprotective ability and limited neurotoxicity. The Examples also suggest that EDR analogue (e.g. NS-1-2)-treated mice, as compared to control/sham treated mice, have decreased weight loss (cachexia), delayed disease onset, and increased survival age in the SOD1-G37R mouse model of ALS. Thus, altogether, these results suggest that the B⁵-EDR analogue(s) are not acutely toxic, and that these tolerated B⁵-EDR analogue(s) may be useful in the treatment and/or prevention of (neurogenerative) diseases, conditions and/or disorders associated with oxidative stress, such as ALS. To this regard, the B5-EDR analogue(s) may be standalone compounds that can function as described and exemplified herein, and/or the B⁵-EDR analogues may be prodrugs of EDR and may impart improved pharmacokinetic properties for EDR, resulting in improved drug-like properties, such as, longer half-life and good oral formulation.

### REFERENCES

1. Accessed Jun. 26, 2021. [Internet]. Available from: https://www.accessdata.fda.gov/drugsatfda_docs/label/2017/2091761bl.pdf
2. Accessed Jun. 26, 2021 [Internet]. Available from: https://www.als.ca/blogs/access-to-therapies-radicava-edaravone-update/
3. Lapchak P A. A critical assessment of edaravone acute ischemic stroke efficacy trials: is edaravone an effective neuroprotective therapy? Expert Opin Pharmacother. 2010 July; 11(10):1753-63.
4. Jaiswal M K. Riluzole and edaravone: A tale of two amyotrophic lateral sclerosis drugs. Med Res Rev. 2019 March; 39(2):733-48.
5. Parakh S, Spencer D M, Halloran M A, Soo K Y, Atkin J D. Redox regulation in amyotrophic lateral sclerosis. Oxid Med Cell Longev. 2013; 2013:408681.
6. Li J, O W, Li W, Jiang Z-G, Ghanbari H A. Oxidative stress and neurodegenerative disorders. Int J Mol Sci [Internet]. 2013 Dec. 16; 14(12):24438-75. Available from: https://pubmed.ncbi.nlm.nih.gov/24351827
7. Rosen D R, Siddique T, Patterson D, Figlewicz D A, Sapp P, Hentati A, et al. Mutations in Cu/Zn superoxide dismutase gene are associated with familial amyotrophic lateral sclerosis. Nature [Internet]. 1993; 362(6415):59-62. Available from: https://doi.org/10.1038/362059a0
8. Battistini S, Giannini F, Greco G, Bibbò G, Ferrera L, Marini V, et al. SOD1 mutations in amyotrophic lateral sclerosis. Results from a multicenter Italian study. J Neurol. 2005 July; 252(7):782-8.
9. Bruijn L I, Houseweart M K, Kato S, Anderson K L, Anderson S D, Ohama E, et al. Aggregation and motor neuron toxicity of an ALS-linked SOD1 mutant independent from wild-type SOD1. Science. 1998 September; 281(5384):1851-4.
10. Uchida K, Kawakishi S. Identification of oxidized histidine generated at the active site of Cu,Zn-superoxide dismutase exposed to H₂O₂. Selective generation of 2-oxo-histidine at the histidine 118. J Biol Chem. 1994 January; 269(4):2405-10.
11. Kurahashi T, Miyazaki A, Suwan S, Isobe M. Extensive Investigations on Oxidized Amino Acid Residues in H₂O₂-Treated Cu,Zn-SOD Protein with LC-ESI-Q-TOF-MS, MS/MS for the Determination of the Copper-Binding Site. J Am Chem Soc [Internet]. 2001 Sep. 1; 123(38):9268-78. Available from: https://doi.org/10.1021/ja0l5953r
12. Vehvilainen P, Koistinaho J, Gundars G. Mechanisms of mutant SOD1 induced mitochondrial toxicity in amyotrophic lateral sclerosis. Front Cell Neurosci. 2014; 8:126.
13. Xu W-C, Liang J-Z, Li C, He Z-X, Yuan H-Y, Huang B-Y, et al. Pathological hydrogen peroxide triggers the fibrillization of wild-type SOD1 via sulfenic acid modification of Cys-111. Cell Death Dis [Internet]. 2018; 9(2):67. Available from: https://doi.org/10.1038/s41419-017-0106-4
14. Chen X, Shang H, Qiu X, Fujiwara N, Cui L, Li X-M, et al. Oxidative modification of cysteine 111 promotes disulfide bond-independent aggregation of SOD1. Neurochem Res. 2012 April; 37(4):835-45.
15. Sies H. Hydrogen peroxide as a central redox signaling molecule in physiological oxidative stress: Oxidative eustress. Redox Biol. 2017 April; 11:613-9.
16. Guareschi S, Cova E, Cereda C, Ceroni M, Donetti E, Bosco D A, et al. An over-oxidized form of superoxide dismutase found in sporadic amyotrophic lateral sclerosis with bulbar onset shares a toxic mechanism with mutant SOD1. Proc Natl Acad Sci USA. 2012 March; 109(13):5074-9.
17. Giordana M T, Piccinini M, Grifoni S, De Marco G, Vercellino M, Magistrello M, et al. TDP-43 redistribution is an early event in sporadic amyotrophic lateral sclerosis. Brain Pathol. 2010 March; 20(2):351-60.
18. Watanabe K, Tanaka M, Yuki S, Hirai M, Yamamoto Y. How is edaravone effective against acute ischemic stroke and amyotrophic lateral sclerosis?J Clin Biochem Nutr [Internet]. 2017/11/11. 2018 January; 62(1):20-38. Available from: https://pubmed.ncbi.nlm.nih.gov/29371752
19. Kikuchi K, Uchikado H, Miyagi N, Morimoto Y, Ito T, Tancharoen S, et al. Beyond neurological disease: new targets for edaravone (Review). Int J Mol Med. 2011 December; 28(6):899-906.
20. Kikuchi K, Takeshige N, Miura N, Morimoto Y, Ito T, Tancharoen S, et al. Beyond free radical scavenging: Beneficial effects of edaravone (Radicut) in various diseases (Review). Exp Ther Med [Internet]. 2012; 3(1):3-8. Available from: https://doi.org/10.3892/etm.2011.352
21. Otomo E, Tohgi H, Kogure K, Hirai S, Takakura K, Terashi A, et al. Effect of a novel free radical scavenger, edaravone (MCI-186), on acute brain infarction: Randomized, placebo-controlled, double-blind study at multicenters. Cerebrovasc Dis. 2003; 15(3):222-9.
22. Rong W-T, Lu Y-P, Tao Q, Guo M, Lu Y, Ren Y, et al. Hydroxypropyl-sulfobutyl-β-cyclodextrin improves the oral bioavailability of edaravone by modulating drug efflux pump of enterocytes. J Pharm Sci. 2014 February; 103(2):730-42.
23. Ma L, Sun J, Peng Y, Zhang R, Shao F, Hu X, et al. Glucuronidation of edaravone by human liver and kidney microsomes: biphasic kinetics and identification of UGT1A9 as the major UDP-glucuronosyltransferase isoform. Drug Metab Dispos. 2012 April; 40(4):734-41.
24. Tanaka M, Sugimura N, Fujisawa A, Yamamoto Y. Stabilizers of edaravone aqueous solution and their action mechanisms. 1. Sodium bisulfite. J Clin Biochem Nutr [Internet]. 2017/10/26. 2017 November; 61(3):159-63. Available from: https://pubmed.ncbi.nlm.nih.gov/29203955
25. Tanaka M, Motomiya S, Fujisawa A, Yamamoto Y. Stabilizers of edaravone aqueous solution and their action mechanisms. 2. Glutathione. J Clin Biochem Nutr. 2017 November; 61(3):164-8.
26. Hunt C D. Dietary boron: progress in establishing essential roles in human physiology. J trace Elem Med Biol organ Soc Miner Trace Elem. 2012 June; 26(2-3):157-60.
27. Silva M P, Saraiva L, Pinto M, Sousa M E. Boronic Acids and Their Analogues in Medicinal Chemistry: Synthesis and Biological Applications. Molecules [Internet]. 2020 Sep. 21; 25(18):4323. Available from: https://pubmed.ncbi.nlm.nih.gov/32967170
28. Penland J G. The importance of boron nutrition for brain and psychological function. Biol Trace Elem Res. 1998; 66(1-3):299-317.
29. Pizzorno L. Nothing Boring About Boron. Integr Med (Encinitas). 2015 August; 14(4):35-48.
30. Kucukkurt I, Ince S, Demirel H H, Turkmen R, Akbel E, Celik Y. The Effects of Boron on Arsenic-Induced Lipid Peroxidation and Antioxidant Status in Male and Female Rats. J Biochem Mol Toxicol. 2015 December; 29(12):564-71.
31. Sogut I, Paltun S O, Tuncdemir M, Ersoz M, Hurdag C. The antioxidant and antiapoptotic effect of boric acid on hepatoxicity in chronic alcohol-fed rats. Can J Physiol Pharmacol. 2018 April; 96(4):404-11.
32. Mitra J, Vasquez V, Hegde P M, Boldogh I, Mitra S, Kent T A, et al. Revisiting Metal Toxicity in Neurodegenerative Diseases and Stroke: Therapeutic Potential. Neurol Res Ther [Internet]. 2014; 1(2):107. Available from: https://pubmed.ncbi.nlm.nih.gov/25717476
33. Lu C-J, Hu J, Wang Z, Xie S, Pan T, Huang L, et al. Discovery of boron-containing compounds as As aggregation inhibitors and antioxidants for the treatment of Alzheimer's disease. Medchemcomm. 2018 November; 9(11):1862-70.
34. Turkez H, Geyikoglu F, Tatar A, Keles M S, Kaplan I. The effects of some boron compounds against heavy metal toxicity in human blood. Exp Toxicol Pathol [Internet]. 2012; 64(1):93-101. Available from: https://www.sciencedirect.com/science/article/pii/S0940299310001107
35. Wang L, Xie S, Ma L, Chen Y, Lu W. 10-Boronic acid substituted camptothecin as prodrug of SN-38. Eur J Med Chem. 2016; 116:84-9.
36. Peng X, Gandhi V. ROS-activated anticancer prodrugs: a new strategy for tumor-specific damage. Ther Deliv. 2012; 3(7):823-33.
37. Duan W, Li X, Shi J, Guo Y, Li Z, Li C. Mutant TAR DNA-binding protein-43 induces oxidative injury in motor neuron-like cell. Neuroscience. 2010 September; 169(4):1621-9.
38. Barber S C, Higginbottom A, Mead R J, Barber S, Shaw P J. An in vitro screening cascade to identify neuroprotective antioxidants in ALS. Free Radic Biol Med [Internet]. 2009 Jan. 30. 2009 Apr. 15; 46(8):1127-38. Available from: https://pubmed.ncbi.nlm.nih.gov/19439221
39. Hemendinger R A, Armstrong E J 3rd, Radio N, Brooks B R. Neurotoxic injury pathways in differentiated mouse motor neuron-neuroblastoma hybrid (NSC-34D) cells in vitro—limited effect of riluzole on thapsigargin, but not staurosporine, hydrogen peroxide and homocysteine neurotoxicity. Toxicol Appl Pharmacol. 2012 January; 258(2):208-15.
40. Tovar-y-Romo L B, Santa-Cruz L D, Tapia R. Experimental models for the study of neurodegeneration in amyotrophic lateral sclerosis. Mol Neurodegener [Internet]. 2009; 4(1):31. Available from: https://doi.org/10.1186/1750-1326-4-31
41. Ito H, Wate R, Zhang J, Ohnishi S, Kaneko S, Ito H, Nakano S, Kusaka H. Treatment with edaravone, initiated at symptom onset, slows motor decline and decreases SOD1 deposition in ALS Mice. Experimental Neurology, 2008, 213(2):448-455.
42. Balimane P V, Han Y-H, Chong S. Current industrial practices of assessing permeability and P-glycoprotein interaction. AAPS J. 2006 January; 8(1):E1-13.
43. Ohashi R, Watanabe R, Esaki T, Taniguchi T, Torimoto-Katori N, Watanabe T, et al. Development of Simplified in Vitro P-Glycoprotein Substrate Assay and in Silico Prediction Models To Evaluate Transport Potential of P-Glycoprotein. Mol Pharm [Internet]. 2019 May 6; 16(5):1851-63. Available from: https://doi.org/10.1021/acs.molpharmaceut.8b01143
44. Di L, Kerns E H, Carter G T. Strategies to assess blood-brain barrier penetration. Expert Opin Drug Discov. 2008 June; 3(6):677-87.
45. Kuhnline Sloan C D, Nandi P, Linz T H, Aldrich J V, Audus K L, Lunte S M. Analytical and biological methods for probing the blood-brain barrier. Annu Rev Anal Chem (Palo Alto Calif). 2012; 5:505-31.
46. Clapham K M, Batsanov A S, Bryce M R, Tarbit B. Trifluoromethyl-substituted pyridyl- and pyrazolylboronic acids and esters: synthesis and Suzuki-Miyaura cross-coupling reactions. Org Biomol Chem. 2009; 7(10):2155-61.
47. Yuen A K, Hutton C A. Deprotection of pinacolyl boronate esters via hydrolysis of intermediate potassium trifluoroborates. Tetrahedron letters. 2005; 46(46):7899-903.
48. Sreedhar B, Venkanna G T, Kumar K B S, Balasubrahmanyam V. Copper (I) oxide catalyzed N-arylation of azoles and amines with arylboronic acid at room temperature under base-free conditions. Synthesis. 2008; 2008(05):795-9.

1. A compound having a structure of Formula I: 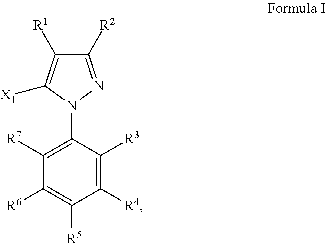
a pharmaceutically-acceptable salt thereof, hydrate thereof, solvate thereof tautomer thereof, geometric isomer thereof, enantiomer thereof, diastereomer thereof, N-oxide thereof, metabolite thereof, isotopomer thereof, isotopologue thereof, prodrug thereof or combination thereof,
wherein:
X₁ is selected from —BR⁸R⁹ or —BR¹⁰R¹¹R¹²;
R¹ to R⁷, R¹⁰, R¹¹, and R¹² are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, —BR¹³R¹⁴, —BR¹⁵R¹⁶R¹⁷, a substituted or unsubstituted carbocyclic group, a substituted or unsubstituted heterocyclic group, a substituted or unsubstituted aromatic group, or a substituted or unsubstituted heteroaromatic group, wherein R¹³ to R¹⁷ are each independently selected from H, a halo group, a hydroxyl group, a cyano group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group or R¹³ and R¹⁴ are taken together to form a substituted or unsubstituted carbocyclic group, or a substituted or unsubstituted heterocyclic group; and
R⁸ and R⁹ are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, a substituted or unsubstituted heteroaromatic group, or are taken together to form a substituted or unsubstituted carbocyclic group, or a substituted or unsubstituted heterocyclic group.

2. The compound according to claim 1, wherein X₁ is —BR⁸R⁹.

3. The compound according to claim 2, wherein at least one of:
i) R⁸ and R⁹ are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, a substituted or unsubstituted heteroaromatic group,
ii) R⁸ and R⁹ are each independently selected from H a halo group, a hydroxyl group, a cyano group, a nitro group, a sulfate group, a substituted or unsubstituted alkyd group a substituted or unsubstituted alkenyl group, a substituted or unsubstituted alkynyl group, a substituted or unsubstituted alkoxy group, —C(O)H, a substituted carbonyl group a substituted carboxyl group, —C(O)OH, a substituted or unsubstituted carbocyclic group, a substituted or unsubstituted heterocyclic group, a substituted or unsubstituted aromatic group, or a substituted or unsubstituted heteroaromatic group;
iii) R⁸ and R⁹ are each independently selected from H, a halo group, a hydroxyl group, a cyano group, a nitro group, a sulfate group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a substituted or unsubstituted C₁-C₆ cycloalkyl group, a substituted or unsubstituted C₁-C₆ heterocyclic group, a substituted or unsubstituted C₁-C₆ aromatic group, or a substituted or unsubstituted C₁-C₆ heteroaromatic group;
iv) R⁸ and R⁹ are each independently selected from a fluoro group, a chloro group, a bromo group, a hydroxyl group, or alkoxy group:
v) R⁸ and R⁹ are taken together to form a substituted or unsubstituted carbocyclic group, or a substituted or unsubstituted heterocyclic group:
vi) R⁸ and R⁹ are taken together to form a substituted or unsubstituted a substituted or unsubstituted heterocyclic group;
vii) R⁸ and R⁹ are taken together to form a substituted or unsubstituted —O(C₂-C₈ alkylene)O— ring
viii) R⁸ and R⁹ are taken together to form —OCH₂CH₂O—, —OC(CH₃)₂CH₂O—, or —OC(CH₃)₂C(CH₃)₂O—;
ix) R⁸ and R⁹ are taken together to form a substituted or unsubstituted —O(C₁-C₂ alkylene)NH(C₁-C₂ alkylene)O— ring; and
x) R⁸ and R⁹ are taken together to form —OCH₂CH₂NHCH₂CH₂O—, —OCH₂CH₂N(CH₃)CH₂CH₂O—, —OCH₂C(CH₃)₂NHCH₂CH₂O—, —OCH₂C(CH₃)₂N(CH₃)CH₂CH₂O—, —OCH₂C(CH₃)₂NHC(CH₃)₂CH₂O, —OC(CH₃)₂CH₂N(CH₃)C(CH₃)₂CH₂O—, or —OC(CH)₂C(CH₃)₂N(CH₃)C(CH₃)₂C(CH)₂O—.

4-12. (canceled)

13. The compound according to claim 1, wherein X₁ is —BR¹⁰R¹¹R¹².

14. The compound according to claim 13, wherein at least one of:
i) R¹⁰, R¹¹ and R¹² are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, a substituted or unsubstituted heteroaromatic group;
ii) R¹⁰, R¹¹ and R¹² are each independently selected from H, a halo group, a hydroxyl group, a cyano group, a nitro group, a sulfate group, a substituted or unsubstituted alkyl group, a substituted or unsubstituted alkenyl group, a substituted or unsubstituted alkynyl group, a substituted or unsubstituted alkoxy group, —C(O)H, a substituted carbonyl group, a substituted carboxyl group, —C(O)OH, a substituted or unsubstituted carbocyclic group, a substituted or unsubstituted heterocyclic group, a substituted or unsubstituted aromatic group, or a substituted or unsubstituted heteroaromatic group;
iii) R¹⁰, R¹¹ and R¹² are each independently selected from H, a halo group, a hydroxyl group, a cyano group, a nitro group, a sulfate group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a substituted or unsubstituted C₁-C₆ cycloalkyl group, a substituted or unsubstituted C₁-C₆ heterocyclic group, a substituted or unsubstituted C₁-C₆ aromatic group, or a substituted or unsubstituted C₁-C₆ heteroaromatic group; and
iv) R¹⁰, R¹¹ and R¹² are each independently selected from a fluoro group, a chloro group, a bromo group, a hydroxyl group, or an alkoxy group.

15-17. (canceled)

18. The compound according to claim 14, wherein any one of;
i) R¹ and R² are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group;
ii) R¹ and R² are each independently selected from H, a halo group, a hydroxyl group, a cyano group, a substituted or unsubstituted amino group, a substituted or unsubstituted alkyl group, a substituted or unsubstituted alkenyl group, a substituted or unsubstituted alkynyl group, a substituted or unsubstituted alkoxy group, a substituted or unsubstituted carbocyclic group, a substituted or unsubstituted heterocyclic group, a substituted or unsubstituted aromatic group, or a substituted or unsubstituted heteroaromatic group;
iii) R¹ and R² are each independently selected from H, a halo group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ cycloalkyl group, a substituted or unsubstituted C₁-C₆ heterocyclic group, a substituted or unsubstituted C₁-C₆ aromatic group, or a substituted or unsubstituted C₁-C₆ heteroaromatic group:
iv) R¹ is H or a substituted or unsubstituted alkyl group and R² is selected from H, a halo group, a hydroxyl group, a cyano group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group; or
v) R¹ is H, —CH₃, —CH₂CH₃, or —CH₂CH₂CH₃ and R² is selected from H, F, Cl, CN, —CH₃, —CH₂F, —CHF₂, or —CF₃.

19-22. (canceled)

23. The compound according to claim 14, wherein any one of:
i) R³ to R⁷ are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, —BR¹³R¹⁴, —BR¹⁵R¹⁶R¹⁷, a substituted or unsubstituted carbocyclic group, a substituted or unsubstituted heterocyclic group, a substituted or unsubstituted aromatic group, or a substituted or unsubstituted heteroaromatic group, wherein R¹³ to R¹⁷ are each independently selected from H, a halo group, a hydroxyl group, a cyano group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group or R¹³ and R¹⁴ are taken together to form a substituted or unsubstituted carbocyclic group, or a substituted or unsubstituted heterocyclic group;
ii) R³ to R⁷ are each independently selected from H, a halo group, a cyano group, a nitro group, a sulfate group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group;
iii) R³ to R⁷ are each independently selected from H, a halo group, a cyano group, a nitro group, a sulfate group, a substituted or unsubstituted alkyl group, a substituted or unsubstituted alkenyl group, a substituted or unsubstituted alkynyl group, a substituted or unsubstituted alkoxy group, —C(O)H, a substituted carbonyl group, a substituted carboxyl group, —C(O)OH, a boronic acid group, a substituted or unsubstituted alkylboronate group, a substituted or unsubstituted carbocyclic group, a substituted or unsubstituted heterocyclic group, a substituted or unsubstituted aromatic group, or a substituted or unsubstituted heteroaromatic group;
iv) R³ to R⁷ are each independently selected from H, a halo group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a boronic acid group, a substituted or unsubstituted C₁-C₆ alkylboronate group, a substituted or unsubstituted C₁-C₆ cycloalkyl group, a substituted or unsubstituted C₁-C₆ heterocyclic group, a substituted or unsubstituted phenyl group, a substituted or unsubstituted alkylheteroaryl group, a substituted or unsubstituted pyridinyl group, or a substituted or unsubstituted pyrrolyl group; and
v) R³, R⁴, R⁶, and R⁷ are each independently selected from H, —CH₃, —CH₂CH₃, or —CH₂CH₂CH₃ and R⁵ is selected from H, F, Cl, CN, —CH₃, —CH₂F, —CHF₂, or —CF₃.

24-27. (canceled)

28. The compound according to claim 1, wherein at least one of:
i) the compound is selected from: 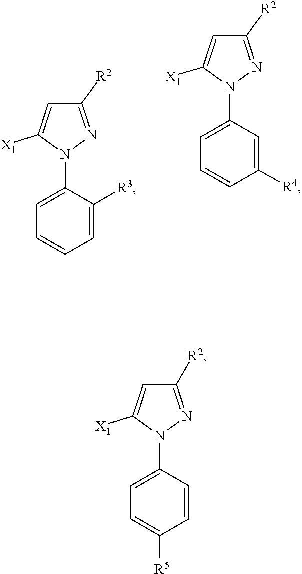
a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof;
ii) the compound is selected from: 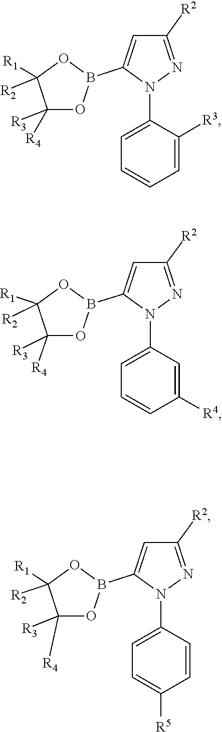
a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof, wherein R₁ to R₄ are each independently selected from any of the groups for R³ to R⁷; and
iii) the compound is selected from: 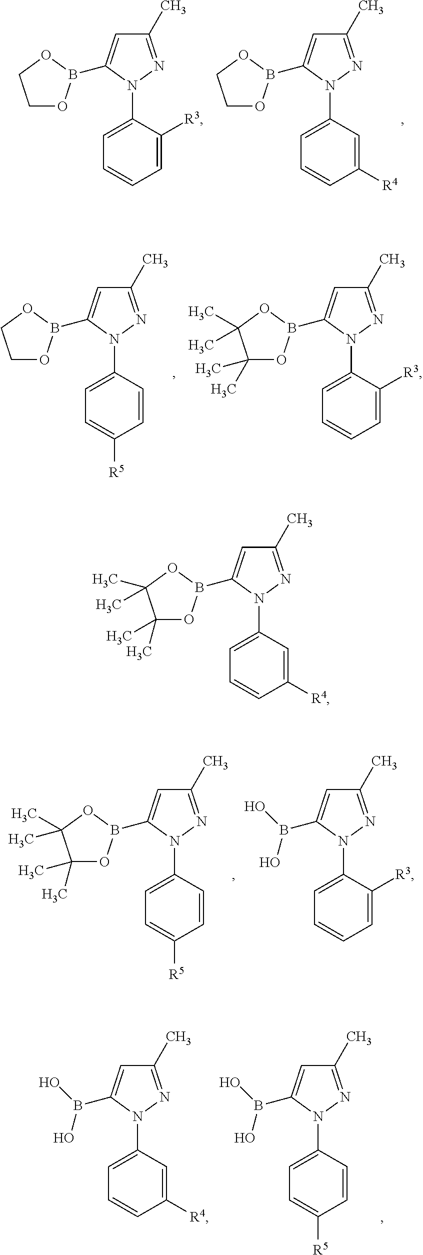 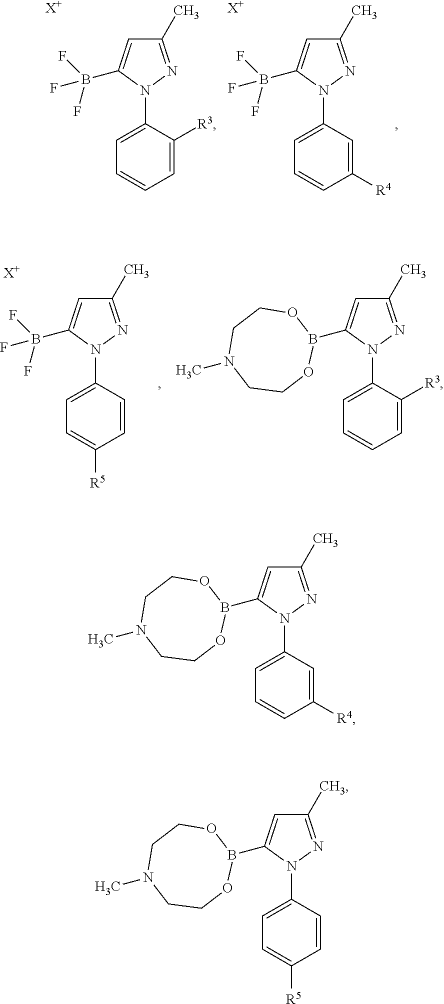
a pharmaceutically-acceptable salt thereof, hydrate thereof, solvate thereof, tautomer thereof geometric isomer thereof, enantiomer thereof, diastereomer thereof, N-oxide thereof, metabolite thereof, isotopomer thereof, isotopologue thereof, prodrug thereof, or combination thereof, wherein X⁺ is any suitable counterion.

29-30. (canceled)

31. The compound according to claim 1, wherein the compound is selected from: 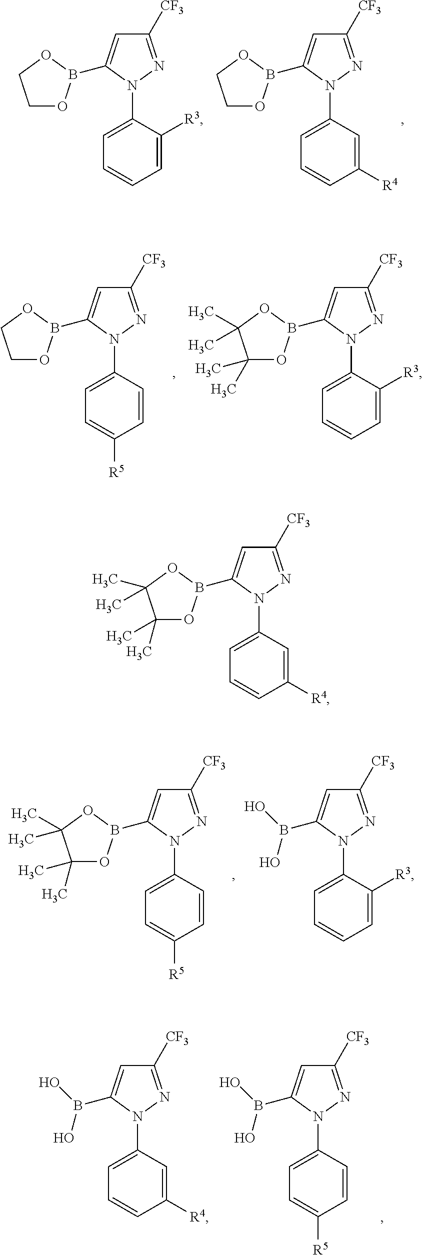 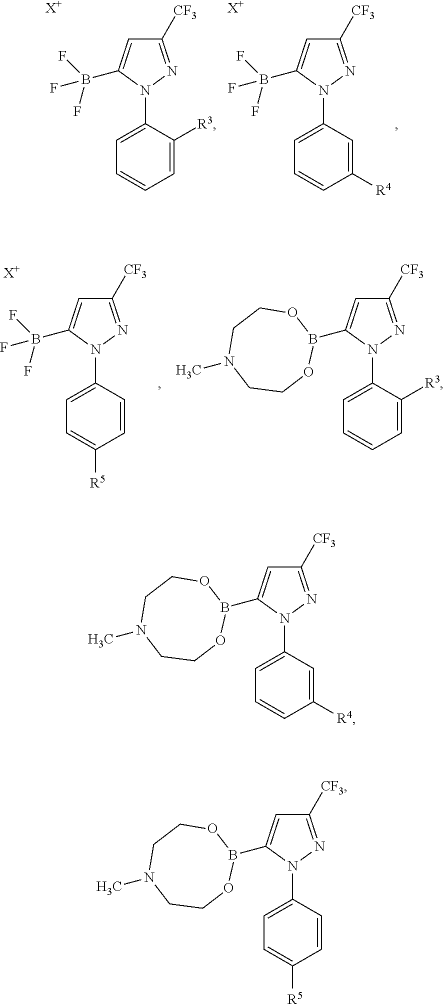
a pharmaceutically-acceptable salt thereof, hydrate thereof, solvate thereof, tautomer thereof, geometric isomer thereof, enantiomer thereof, diastereomer thereof, N-oxide thereof, metabolite thereof, isotopomer thereof, isotopologue thereof, prodrug thereof, or combination thereof, wherein X⁺ is any suitable counterion.

32-33. (canceled)

34. The compound according to claim 1, wherein at least one of:
i) the compound is a pharmaceutically acceptable salt;
ii) the compound act as a metal chelator;
iii) the compound acts as an antioxidant;
iv) the compound reduces oxidative stress;
v) the compound reduces oxidative damage/oxidative stress by scavenging reactive oxygen species (ROS) and/or reactive nitrogen species (RNS);
vi) the compound can reduce reactive oxygen species (ROS) and/or reactive nitrogen species (RNS) in vitro;
vii) the compound can reduce reactive oxygen species (ROS) and/or reactive nitrogen species (RNS) in vivo; and
viii) the ROS and/or RNS comprise free radicals and/or oxidants,
wherein the free radicals and/or oxidants are selected from hydroxyl radical (HO·), Superoxide radical anion radical (O2·-), nitric oxide (NO·), nitrogen dioxide (NO₂·), peroxyl (ROO·) and lipid peroxyl (LOO·), hydrogen peroxide (H₂O₂), ozone (O₃), singlet oxygen (¹O₂), hypochlorous acid (HOCl), hydroperoxide nucleophile (⁻OOH), nitrous acid (HNO₂), dinitrogen trioxide (N₂O₃), and lipid peroxide (LOOH), optionally,
the free radicals and/or oxidants is hydrogen peroxide (H₂O₂).

35-43. (canceled)

44. The compound according to claim 1, wherein at least one of:
i) the compound can react with hydrogen peroxide in vivo to produce edaravone (EDR);
ii) the compound can react with hydrogen peroxide in vitro to produce edaravone (EDR);
iii) the compound is a prodrug of edaravone (EDR);
iv) the compound is an antioxidant and prodrug of edaravone (EDR);
v) the compound is an amyotrophic lateral sclerosis (ALS) therapeutic in vivo; and
vi) the compound is a therapeutic for prevention and/or treatment of a disease, condition and/or disorder associated with oxidative stress.

45-49. (canceled)

50. A pharmaceutical composition comprising the compound according to claim 1, optionally, for use in the prevention and/or treatment of a disease, condition and/or disorder associated with oxidative stress.

51-52. (canceled)

53. The compound or composition of claim 50 wherein the oxidative stress is caused by reactive oxygen species (ROS) and/or reactive nitrogen species (RNS), optionally,
wherein the ROS and/or RNS comprise free radicals and/or oxidants
wherein the free radicals and/or oxidants are selected from hydroxyl radical (HO·), Superoxide radical anion radical (O2·-), nitric oxide (NO·) nitrogen dioxide (NO₂·), peroxyl (ROO·) and lipid peroxyl (LOO·), hydrogen peroxide (H₂O₂), ozone (O₃), singlet oxygen (¹O₂), hypochlorous acid (HOCl), hydroperoxide nucleophile (⁻OOH), nitrous acid (HNO₂), dinitrogen trioxide (N₂O₃), and lipid peroxide (LOOH), optionally,
the free radicals and/or oxidants is hydrogen peroxide (H₂O₂).

54-56. (canceled)

57. The compound or composition according to claim 50, wherein at least one of:
i) the disease, condition and/or disorder associated with oxidative stress is selected from neurodegenerative diseases, disorders and/or conditions, muscle diseases, disorders and/or conditions, vascular diseases, disorders and/or conditions, systemic inflammatory diseases, disorders, and/or conditions, local inflammatory diseases, disorders, and/or conditions, metabolic syndrome, cardiovascular diseases, disorders, and/or conditions, autoimmune diseases, disorders, and/or conditions, inflammatory lung diseases, disorders, and/or conditions, kidney diseases, disorders, and/or conditions, liver diseases, disorders, and/or conditions, digestive diseases, disorders, and/or conditions, aging, disorders and/or conditions, viral infectious diseases, disorders, and/or conditions, cancer, and sepsis/septic shock;
ii) the neurodegenerative disease, condition and/or disorder is associated with motor dysfunction;
iii) the neurodegenerative disease, condition and/or disorder associated with motor dysfunction is amyotrophic lateral sclerosis (ALS);
iv the ALS is familial ALS;
v) the ALS is sporadic ALS;
vi) the ALS is caused by a mutation in the superoxide dismutase 1 (SOD1) gene or TARDBP gene; and
vii) the compound or composition delays the onset of ALS.

58-63. (canceled)

64. The compound or composition according to claim 50, wherein the compound or composition has an improved therapeutic index for ALS as compared to edaravone (EDR), optionally, wherein at least one of:
i) the therapeutic index is a measure of one or more of the following symptoms muscle weakness, muscle wasting (atrophy), weight loss (cachexia), muscle fasciculations, muscle spasticity, slowness of movement, poor balance, incoordination, alterations in vocal quality, dysarthria, dysphagia, incomplete eye closure, drooling, pseudobulbar affect, and/or premature death;
ii) the improved therapeutic index is measured by increased survival/lifespan in the subject receiving the compound or composition as compared to the subject receiving edaravone (EDR);
iii) the improved therapeutic index is measured by increased survival/lifespan in the subject receiving the compound or composition as compared to the subject receiving edaravone (EDR);
iv) the improved therapeutic index is measured by a lower percentage of weight loss (cachexia), in the subject receiving the compound or composition as compared to a subject receiving edaravone (EDR); and
v) the compound or composition has an improved pharmacokinetics as compared to edaravone (EDR), optionally,
the improved pharmacokinetics comprises increased bioavailability of the compound or composition as compared to edaravone (EDR).

65-70. (canceled)

71. A method for preventing and/or treating a disease, condition and/or disorder associated with oxidative stress, comprising administering to a mammal a therapeutically effective amount of the compound according to claim 1.

72. The method according to claim 71, wherein the oxidative stress is caused by reactive oxygen species (ROS) and/or reactive nitrogen species (RNS), optionally,
wherein the ROS and/or RNS comprise free radicals and/or oxidants,
wherein the free radicals and/or oxidants are selected from hydroxyl radical (HO·), Superoxide radical anion radical (O2·-), nitric oxide (NO·), nitrogen dioxide (NO₂·), peroxyl (ROO·) and lipid peroxyl (LOO·), hydrogen peroxide (H₂O₂), ozone (O₃), singlet oxygen (¹O₂), hypochlorous acid (HOCl), hydroperoxide nucleophile (⁻OOH), nitrous acid (HNO₂), dinitrogen trioxide (N₂O₃), and lipid peroxide (LOOH); optionally,
the free radicals and/or oxidants is hydrogen peroxide (H₂O₂).

73-75. (canceled)

76. The method according to claim 71, wherein at least one of:
i) the disease, condition and/or disorder associated with oxidative stress is selected from neurodegenerative diseases, disorders and/or conditions, muscle diseases, disorders and/or conditions, vascular diseases, disorders and/or conditions, systemic inflammatory diseases, disorders, and/or conditions, local inflammatory diseases, disorders, and/or conditions, metabolic syndrome, cardiovascular diseases, disorders, and/or conditions, autoimmune diseases, disorders, and/or conditions, inflammatory lung diseases, disorders, and/or conditions, kidney diseases, disorders, and/or conditions, liver diseases, disorders, and/or conditions, digestive diseases, disorders, and/or conditions, aging, disorders and/or conditions, viral infectious diseases, disorders, and/or conditions, cancer, and sepsis/septic shock;
ii) the neurodegenerative disease, condition and/or disorder is associated with motor dysfunction;
iii) the neurodegenerative disease, condition and/or disorder associated with motor dysfunction is amyotrophic lateral sclerosis (ALS);
iv) the ALS is familial ALS;
v) the ALS is sporadic ALS;
vi) the ALS is caused by a mutation in the superoxide dismutase 1 (SOD1) gene or TARDBP gene; and
vii) the compound or composition delays the onset of ALS.

77-82. (canceled)

83. The method according to claim 71, wherein the compound or composition has an improved therapeutic index for ALS as compared to edaravone (EDR), optionally, wherein at least one of:
i) the therapeutic index is a measure of one or more of the following symptoms muscle weakness, muscle wasting (atrophy), weight loss (cachexia), muscle fasciculations, muscle spasticity, slowness of movement, poor balance, incoordination, alterations in vocal quality, dysarthria, dysphagia, incomplete eye closure, drooling, pseudobulbar affect, and/or premature death;
ii) the improved therapeutic index is measured by increased survival/lifespan in the subject receiving the compound or composition as compared to the subject receiving edaravone (EDR);
iii) the improved therapeutic index is measured by increased motor function in the subject receiving the compound or composition as compared to a subject receiving edaravone (EDR);
iv) the improved therapeutic index is measured by a lower percentage of weight loss (cachexia), in the subject receiving the compound or composition as compared to a subject receiving edaravone (EDR);
v) the compound or composition has an improved pharmacokinetics as compared to edaravone (EDR); and
vi) the improved pharmacokinetics comprises increased bioavailability of the compound or composition as compared to edaravone (EDR).

84-112. (canceled)

113. The compound according to claim 3, wherein any one of:
i) R¹ and R² are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group;
ii) R¹ and R² are each independently selected from H, a halo group, a hydroxyl group, a cyano group, a substituted or unsubstituted amino group, a substituted or unsubstituted alkyl group, a substituted or unsubstituted alkenyl group, a substituted or unsubstituted alkynyl group, a substituted or unsubstituted alkoxy group, a substituted or unsubstituted carbocyclic group, a substituted or unsubstituted heterocyclic group, a substituted or unsubstituted aromatic group, or a substituted or unsubstituted heteroaromatic group;
iii) R¹ and R² are each independently selected from H, a halo group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ cycloalkyl group, a substituted or unsubstituted C₁-C₆ heterocyclic group, a substituted or unsubstituted C₁-C₆ aromatic group, or a substituted or unsubstituted C₁-C₆ heteroaromatic group;
iv) R¹ is H or a substituted or unsubstituted alkyl group and R² is selected from H, a halo group, a hydroxyl group, a cyano group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group; or
v) R¹ is H, —CH₃, —CH₂CH₃, or —CH₂CH₂CH₃ and R² is selected from H, F, Cl, CN, —CH₃, —CH₂F, —CHF₂, or —CF₃.

114. The compound according to claim 3, wherein any one of:
i) R³ to R⁷ are each independently selected from H, a halo group, a hydroxyl group, a cyano group, a nitro group, a thiol group, a sulfonyl group, a sulfate group, a substituted or unsubstituted amino group, a substituted or unsubstituted hydrocarbon group, a substituted or unsubstituted heterogeneous group, —BR¹³R¹³, —BR¹⁵R¹⁶R¹⁷, a substituted or unsubstituted carbocyclic group, a substituted or unsubstituted heterocyclic group, a substituted or unsubstituted aromatic group, or a substituted or unsubstituted heteroaromatic group, wherein R¹³ to R¹⁷ are each independently selected from H, a halo group, a hydroxyl group, a cyano group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group or R¹³ and R¹⁴ are taken together to form a substituted or unsubstituted carbocyclic group, or a substituted or unsubstituted heterocyclic group;
ii) R³ to R⁷ are each independently selected from H, a halo group, a cyano group, a nitro group, a sulfate group, a substituted or unsubstituted hydrocarbon group, a substituted or unsubstituted heterogeneous group, a substituted or unsubstituted carbocyclic group, a substituted or unsubstituted heterocyclic group, a substituted or unsubstituted aromatic group, or a substituted or unsubstituted heteroaromatic group;
iii) R³ to R⁷ are each independently selected from H, a halo group, a cyano group, a nitro group, a sulfate group, a substituted or unsubstituted alkyl group, a substituted or unsubstituted alkenyl group, a substituted or unsubstituted alkynyl group, a substituted or unsubstituted alkoxy group, —C(O)H, a substituted carbonyl group, a substituted carboxyl group, —C(O)OH, a boronic acid group, a substituted or unsubstituted alkylboronate group, a substituted or unsubstituted carbocyclic group, a substituted or unsubstituted heterocyclic group, a substituted or unsubstituted aromatic group, or a substituted or unsubstituted heteroaromatic group;
iv) R³ to R⁷ are each independently selected from H, a halo group, a substituted or unsubstituted C₁-C₆ alkyl group, a C₁-C₆ haloalkyl group, a substituted or unsubstituted (C₁-C₆ alkyl)hetero(C₁-C₆ alkyl) group, a substituted or unsubstituted C₁-C₆ alkoxy group, —C(O)H, a substituted or unsubstituted C₁-C₆ alkylcarbonyl group, a substituted or unsubstituted (C₁-C₆ alkyl)carbonyl(C₁-C₆ alkyl) group, a substituted or unsubstituted C₁-C₆ alkyl-C(O)O— group, a substituted or unsubstituted C₁-C₆ alkyl-O—C(O)— group, a substituted or unsubstituted C₁-C₆ alkyl-O—C(O)—C₁-C₆ alkylene group, —C(O)OH, a boronic acid group, a substituted or unsubstituted C₁-C₆ alkylboronate group, a substituted or unsubstituted C₁-C₆ cycloalkyl group, a substituted or unsubstituted C₁-C₆ heterocyclic group, a substituted or unsubstituted phenyl group, a substituted or unsubstituted alkylheteroaryl group, a substituted or unsubstituted pyridinyl group, or a substituted or unsubstituted pyrrolyl group; and
v) R³, R⁴, R⁶, and R⁷ are each independently selected from H, —CH₃, —CH₂CH₃, or —CH₂CH₂CH₃ and R⁵ is selected from H, F, Cl, CN, —CH₃, —CH₂F, —CHF₂, or —CF₃.
